SEQUENCE LISTING (3 sequences)

SEQ ID NO: 1 - protein, 141 aa, Artificial Sequence
  PDIa
MGSSHHHHHHSSGLVPRGSHMDAPEEEDHVLVLRKSNFAEALAAHKYLLVEFYAPWCGHCKALAPEYAKAAGKLKAEGSEIRLAKVDATEESDLAQQYGVRGYPTIKFFRNGDTASPKEYTAGREADDIVNWLKKRTGPAA

SEQ ID NO: 2 - protein, 15 aa, Homo sapiens
YLLVEFYAPWCGHCK

SEQ ID NO: 3 - protein, 491 aa, Homo sapiens
DAPEEEDHVLVLRKSNFAEALAAHKYLLVEFYAPWCGHCKALAPEYAKAAGKLKAEGSEIRLAKVDATEESDLAQQYGVRGYPTIKFFRNGDTASPKEYTAGREADDIVNWLKKRTGPAATTLPDGAAAESLVESSEVAVIGFFKDVESDSAKQFLQAAEAIDDIPFGITSNSDVFSKYQLDKDGVVLFKKFDEGRNNFEGEVTKENLLDFIKHNQLPLVIEFTEQTAPKIFGGEIKTHILLFLPKSVSDYDGKLSNFKTAAESFKGKILFIFIDSDHTDNQRILEFFGLKKEECPAVRLITLEEEMTKYKPESEELTAERITEFCHRFLEGKIKPHLMSQELPEDWDKQPVKVLVGKNFEDVAFDEKKNVFVEFYAPWCGHCKQLAPIWDKLGETYKDHENIVIAKMDSTANEVEAVKVHSFPTLKFFPASADRTVIDYNGERTLDGFKKFLESGGQDGAGDDDDLEDLEEAEEPDMEEDDDQKAVKDEL

(19) United States
(12) Patent Application Publication
(10) Pub. No.: US 2018/0092908 A1
(43) Pub. Date: Apr. 5, 2018

(54) SMALL MOLECULE OXIDIZERS OF PDI AND THEIR USE

(71) Applicant: THE TRUSTEES OF COLUMBIA UNIVERSITY IN THE CITY OF NEW YORK, New York, NY (US)

(72) Inventors: Brent R. Stockwell, New York, NY (US); Anna Kaplan, New York, NY (US)

(21) Appl. No.: 15/655,712

(22) Filed: Jul. 20, 2017

Related U.S. Application Data
(63) Continuation-in-part of application No. PCT/US16/14149, filed on Jan. 20, 2016.
(60) Provisional application No. 62/105,656, filed on Jan. 20, 2015.

(51) Int. Cl. A61K 31/496; A61P 25/28; A61P 25/16; A61P 35/00; A61P 31/18; A61P 7/00; C07D 417/12; C07D 275/04; C07D 261/20; C07D 417/06; A61K 31/454; A61K 31/541; A61K 31/506; A61K 31/5377; A61K 31/428

(52) U.S. Cl. CPC A61K 31/496; A61K 31/428; A61P 25/16; A61P 35/00; A61P 31/18; A61P 7/00; C07D 417/12; C07D 275/04; C07D 261/20; C07D 417/06; A61K 31/454; A61K 31/541; A61K 31/506; A61K 31/5377; A61P 25/28

(57) ABSTRACT

The present invention provides a method for treating or ameliorating the effects of a neurodegenerative disorder in a subject in need thereof. The method includes, for example, administering to the subject an effective amount of a compound selected from:

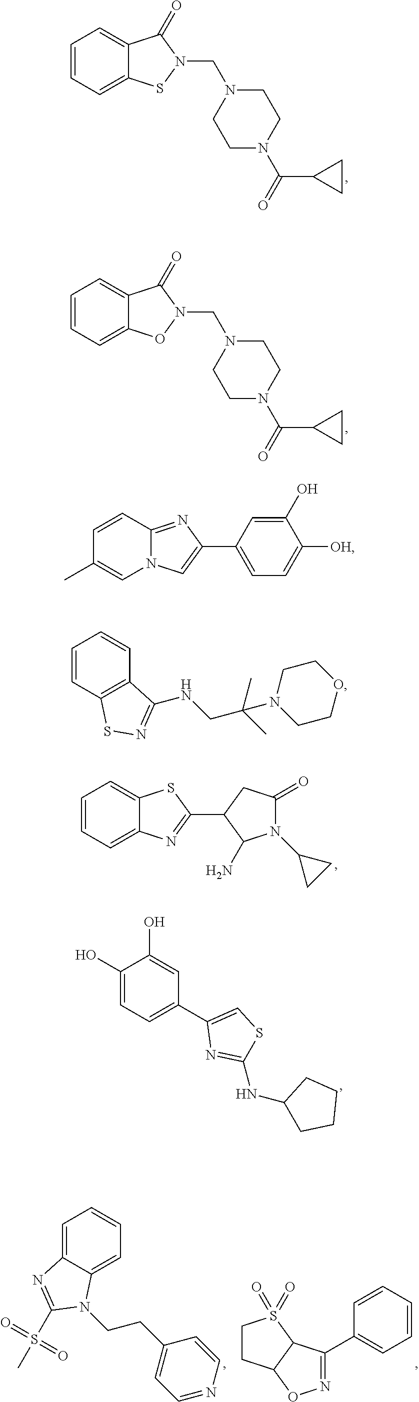

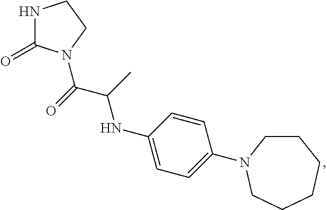 [SMILES: CC(Nc1ccc(N2CCCCCC2)cc1)C(=O)N1CCNC1=O]

combinations thereof, or an N-oxide, crystalline form, hydrate thereof, or a pharmaceutically acceptable salt thereof. The present invention also provides a method for treating or ameliorating the effects of a condition associated with increased protein disulfide isomerase (PDI) activity and a method of modulating PDI activity in a cell. The present invention also provides compounds, salts, compositions and kits useful for the provided methods.

### CROSS REFERENCE TO RELATED APPLICATIONS

This application is a continuation in part of PCT international application no. PCT/US2016/014149, filed Jan. 20, 2016, which claims benefit of U.S. Patent Application Ser. No. 62/105,656, filed on Jan. 20, 2015, which applications are incorporated by reference herein in their entireties.

### GOVERNMENT FUNDING

This invention was made with government support under grant nos. CA097061, GM085081, and GM008281 awarded by the National Institutes of Health. The government has certain rights in the invention.

### INCORPORATION BY REFERENCE OF SEQUENCE LISTING

This application contains references to amino acids and/or nucleic acid sequences that have been filed concurrently herewith as sequence listing text file “0391835_ST25.txt”, file size of 9 KB, created on Jan. 20, 2015. The aforementioned sequence listing is hereby incorporated by reference in its entirety pursuant to 37 C.F.R. § 1.52(e)(5).

### FIELD OF THE INVENTION

The present invention relates to modulators of protein disulfide isomerase (PDI). More particularly, the present invention provides small molecule inhibitors of PDI which are neuroprotective.

### BACKGROUND OF THE INVENTION

Neurodegenerative disorders constitute a class of diseases that express characteristic misfolded proteins that aggregate and induce neuronal toxicity and death. Huntington disease (HD) is one such fatal protein misfolding disease that afflicts primarily medium spiny neurons in the striatum. HD is caused by expansion to more than 36 CAG trinucleotide repeats in the huntingtin gene. These CAG repeats translate into an expanded polyglutamine tract in the huntingtin protein, causing it to aggregate, and drive neuronal dysfunction and progressive neuronal loss. Currently there is no therapeutic avenue that can delay or stop the progression of the disease. In this context, there is a need to develop therapeutics and drug targets that can prevent or delay pathogenesis in neurodegenerative diseases, such as HD, involving protein misfolding.

Previously, it was reported that modulation of protein disulfide isomerase (PDI) by small molecules is beneficial in cell and brain slice models of HD (Hoffstrom et al., 2010). PDI is a thiol-oxidoreductase chaperone protein that is responsible for the isomerization, reduction, and oxidation of non-native disulfide bonds in unfolded proteins entering the endoplasmic reticulum (ER). Structurally, PDI consists of four domains with a thioredoxin fold: a, b, b′ and a′, an extended C-terminus with KDEL ER retention sequence, and an interdomain linker x between the b′ and a′ domains. The a and a′ domains are catalytically active, contain the WCGHC active site and independently can perform oxidation and reduction reactions (Darby & Creighton, 1995). However, all four domains are needed to achieve the isomerization and chaperone activity of PDI. Besides its catalytic role involving thiols and disulfides, PDI also serves an essential structural role as the beta subunit of prolyl-4-hydroxylase (Koivu et al., 1987) and as a microsomal triglyceride transfer protein (Wetterau et al., 1990).

PDI is upregulated in mouse models of, and in brains of patients with, neurological protein folding diseases (Yoo et al., 2002; Colla et al., 2012; Atkin et al., 2008). In addition, it has also been implicated in a number of cancers (Xu et al., 2012; Hashida et al., 2011; Lovat et al., 2008), HIV-1 pathogenesis (Barbouche et al., 2003), and blood clot formation (Cho et al., 2008), suggesting the growing importance of understanding this enzyme. One challenge has been the lack of available drug-like inhibitors, especially for in vivo evaluation in neurodegenerative disease models. Reported inhibitors of PDI are either (i) irreversible binders to the catalytic site cysteines (Hoffstrom et al., 2010; Xu et al., 2012; Ge et al., 2013), (ii) not cell permeable, because they were designed for the inhibition of extracellular PDI (Jasuja et al., 2012; Khan et al., 2011) or (iii) nonselective hormones and antibiotics, such as estrone and bacitracin, that act broadly on multiple target proteins (Khan et al., 2011; Karala & Ruddock, 2010). Irreversible inhibitors, although having promise in ovarian cancer, have mechanism-based toxicity that is not likely well tolerated in neurons. PDI is an essential protein, whose irreversible genetic silencing is cytotoxic to cells and probably in animal models as well, since no genetic PDI null has been generated. The related PDI A3 (ERp57) protein knockout resulted in embryonic lethality in mice (Garbi et al., 2006). Thus, irreversible inhibitors of PDI may exhibit the same level of cytotoxicity in vivo. It was hypothesized that reversible, non-covalent inhibitors of PDI might exhibit a therapeutic window upon PDI inhibition, and would have improved pharmaceutical properties. The present invention is directed towards these and other needs.

### SUMMARY OF THE INVENTION

In the present invention, the inventors have discovered a neuroprotective, reversible modulator of PDI that has nanomolar potency, high in vitro stability in liver microsomes and blood plasma, and is protective for medium spiny neurons in a brain slice model for HD. This scaffold represents a class of reversible modulators of PDI that can probe its potential as a drug target for neurological diseases with misfolded proteins.

The present invention provides a method for treating or ameliorating the effects of a neurodegenerative disorder in a subject in need thereof comprising administering to the subject an effective amount of a compound selected from the group consisting of:

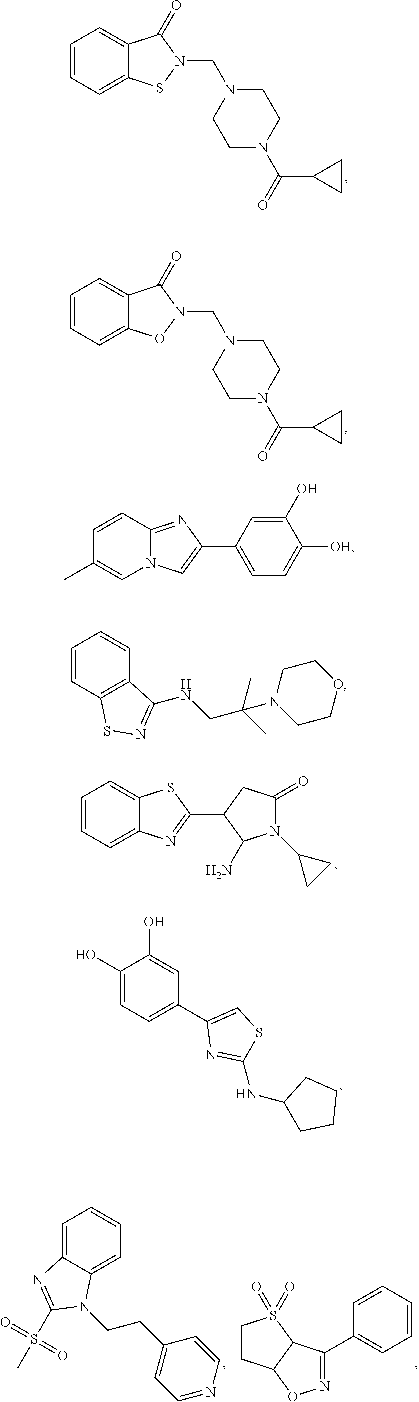

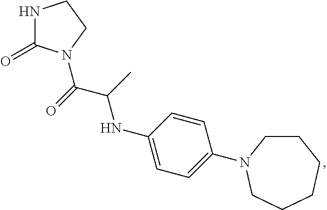 [SMILES: CC(Nc1ccc(N2CCCCCC2)cc1)C(=O)N1CCNC1=O]

combinations thereof,
or an N-oxide, crystalline form, hydrate thereof, or a pharmaceutically acceptable salt thereof.

The present invention also provides a method for treating or ameliorating the effects of a condition associated with increased protein disulfide isomerase (PDI) activity in a subject in need thereof comprising administering to the subject an effective amount of a compound selected from the group consisting of:

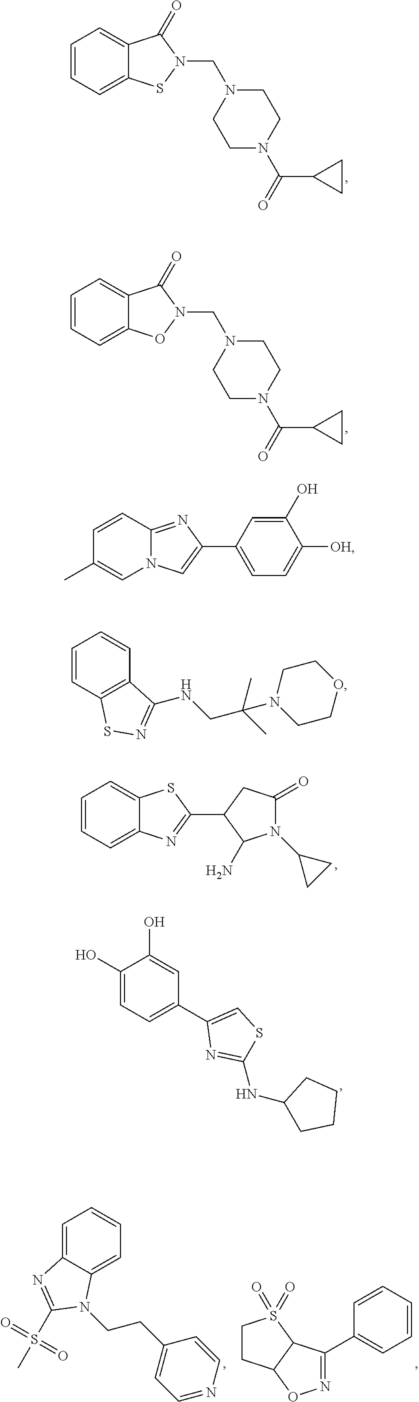

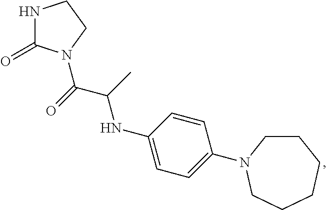 [SMILES: CC(Nc1ccc(N2CCCCCC2)cc1)C(=O)N1CCNC1=O]

combinations thereof, or an N-oxide, crystalline form, hydrate thereof, or a pharmaceutically acceptable salt thereof.

The present invention also provides a method of modulating PDI activity in a cell comprising contacting the cell with an effective amount of a compound selected from the group consisting of:

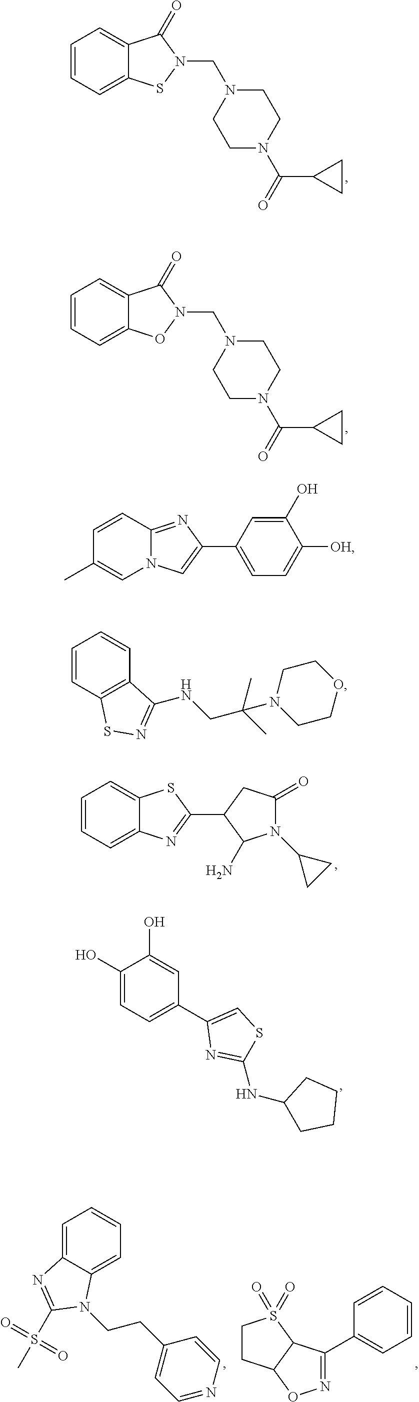

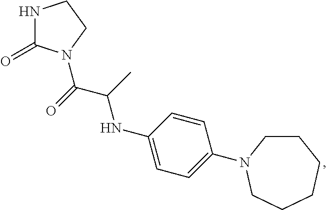 [SMILES: CC(Nc1ccc(N2CCCCCC2)cc1)C(=O)N1CCNC1=O]

combinations thereof,
or an N-oxide, crystalline form, hydrate thereof, or a pharmaceutically acceptable salt thereof.

The present invention also provides a method for treating or ameliorating the effects of a neurodegenerative disorder in a subject in need thereof comprising administering to the subject an effective amount of a compound selected from the group consisting of formula (I)

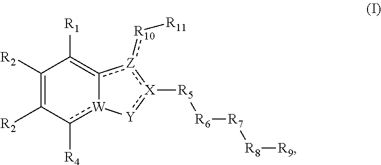

formula (II)

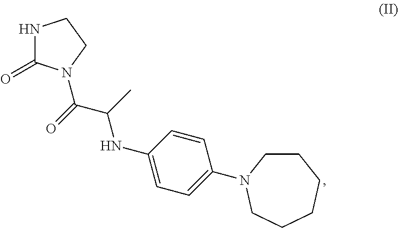 [SMILES: CC(Nc1ccc(N2CCCCCC2)cc1)C(=O)N1CCNC1=O]

formula (III)

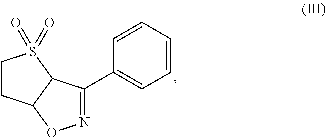 [SMILES: O=S1(=O)CCC2ON=C(c3ccccc3)C21]

and formula (IV):

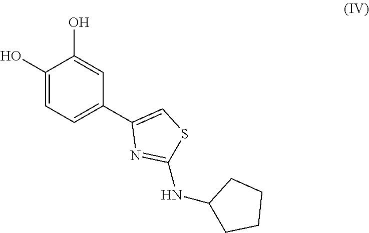 [SMILES: Oc1ccc(-c2csc(NC3CCCC3)n2)cc1O]

wherein a dashed line indicates the presence of an optional double bond,
wherein W, X, Y and Z are independently selected from the group consisting of C, N, S and O,
wherein R₁, R₂, R₃, and R₄ are independently selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₅, R₆, R₇, R₈ and R₁₀ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S), wherein R₉ and R₁₁ are independently selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S), wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof, or an N-oxide, crystalline form, hydrate thereof, or a pharmaceutically acceptable salt thereof.

The present invention also provides a method of treating or ameliorating the effects of a condition associated with increased protein disulfide isomerase (PDI) activity in a subject in need thereof comprising administering to the subject an effective amount of a compound selected from the group consisting of formula (I)

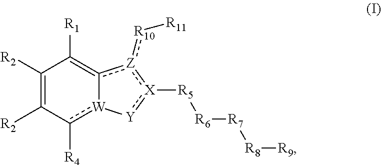

formula (II)

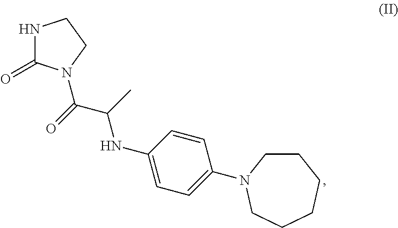 [SMILES: CC(Nc1ccc(N2CCCCCC2)cc1)C(=O)N1CCNC1=O]

formula (III)

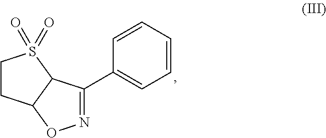 [SMILES: O=S1(=O)CCC2ON=C(c3ccccc3)C21]

and formula (IV):

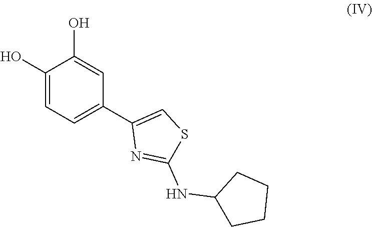 [SMILES: Oc1ccc(-c2csc(NC3CCCC3)n2)cc1O]

wherein a dashed line indicates the presence of an optional double bond,
wherein W, X, Y and Z are independently selected from the group consisting of C, N, S and O,
wherein R₁, R₂, R₃, and R₄ are independently selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₅, R₆, R₇, R₈ and R₁₀ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S), wherein R₉ and R₁₁ are independently selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S), wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof, or an N-oxide, crystalline form, hydrate thereof, or a pharmaceutically acceptable salt thereof.

The present invention also provides a method of modulating PDI activity in a cell comprising contacting the cell with an effective amount of a compound selected from the group consisting of formula (I)

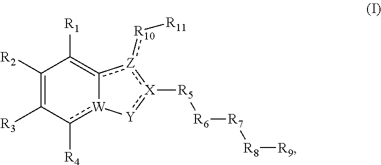

formula (II)

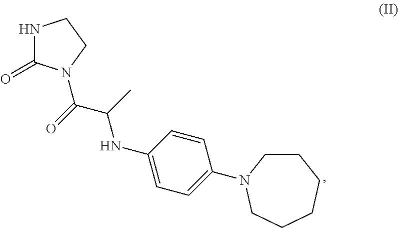 [SMILES: CC(Nc1ccc(N2CCCCCC2)cc1)C(=O)N1CCNC1=O]

formula (III)

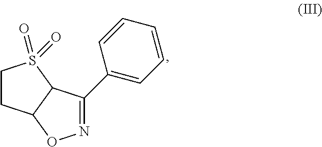 [SMILES: O=S1(=O)CCC2ON=C(c3ccccc3)C21]

and formula (IV):

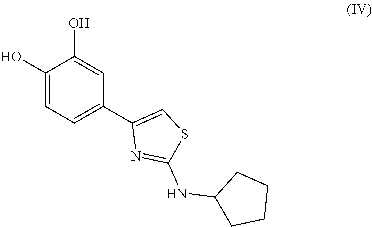 [SMILES: Oc1ccc(-c2csc(NC3CCCC3)n2)cc1O]

wherein a dashed line indicates the presence of an optional double bond,
wherein W, X, Y and Z are independently selected from the group consisting of C, N, S and O,
wherein R₁, R₂, R₃, and R₄ are independently selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₅, R₆, R₇, R₈ and R₁₀ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₉ and R₁₁ are independently selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof, or an N-oxide, crystalline form, hydrate thereof, or a pharmaceutically acceptable salt thereof.

The present invention also provides a compound having the formula (I)

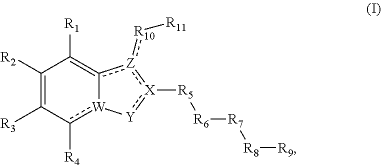

wherein a dashed line indicates the presence of an optional double bond,
wherein W, X, Y and Z are independently selected from the group consisting of C, N, S and O,
wherein R₁, R₂, R₃, and R₄ are independently selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₅, R₆, R₇, R₈ and R₁₀ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S), wherein R₉ and R₁₁ are independently selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof, with the proviso that the compound is not

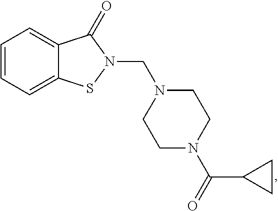 [SMILES: O=C(C1CC1)N1CCN(Cn2sc3ccccc3c2=O)CC1]

or an N-oxide, crystalline form, hydrate thereof, or a pharmaceutically acceptable salt thereof.

The present invention also provides a compound having the formula (Ia)

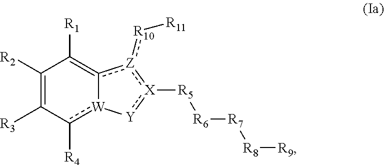

wherein a dashed line indicates the presence of an optional double bond,
wherein W, X, and Z are independently selected from the group consisting of C, N, S and O,
wherein Y is selected from the group consisting of C, N, Se, S and O, or another group VI atom,
wherein R₁, R₂, R₃, and R₄ are independently selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₅, R₆, R₇, R₈ and R₁₀ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S), wherein R₉ and R₁₁ are independently selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof, with the proviso that the compound is not

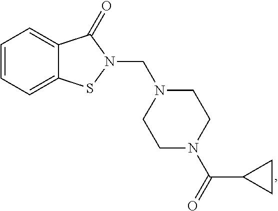 [SMILES: O=C(C1CC1)N1CCN(Cn2sc3ccccc3c2=O)CC1]

or an N-oxide, crystalline form, hydrate thereof, or a pharmaceutically acceptable salt thereof.

The present invention also provides a compound having the formula (II)

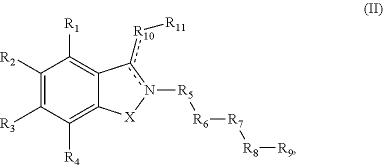

wherein a dashed line indicates the presence of an optional double bond,
wherein X is selected from the group consisting of S and Se,
wherein R₁, R₂, R₃, and R₄ are independently selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₅ is selected from the group consisting of C₁₋₄alkyl, C(O)NH, C(O), C(O)O, NH and O,
wherein R₆, is -(optionally substituted mono- or polycyclic group containing 3 to 20 carbon atoms and optionally 1 to 4 heteroatoms selected from O, N and S),
wherein R₇ and R₈ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₉ is selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₁₀ is selected from the group consisting of no atom and O,
wherein R₁₁ is selected from the group consisting of O and -(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof, with the proviso that the compound is not

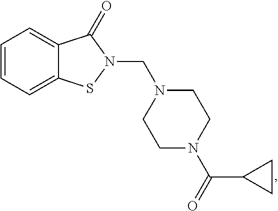 [SMILES: O=C(C1CC1)N1CCN(Cn2sc3ccccc3c2=O)CC1]

or an N-oxide, crystalline form, hydrate thereof, or a pharmaceutically acceptable salt thereof.

The present invention also provides a compound having the formula (III)

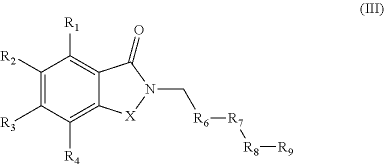

wherein X is selected from the group consisting of S and Se,
wherein R₆, is selected from the group consisting of the group consisting of phenyl,

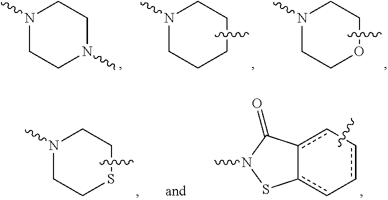

wherein a wavy line indicates an attachment point to the molecule,
wherein R₇ and R₈ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₉ is selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof, with the proviso that the compound is not

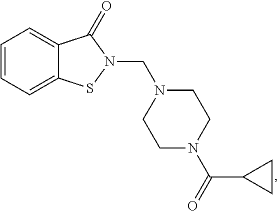 [SMILES: O=C(C1CC1)N1CCN(Cn2sc3ccccc3c2=O)CC1]

or an N-oxide, crystalline form, hydrate thereof, or a pharmaceutically acceptable salt thereof.

The present invention also provides a composition comprising a compound of the present invention and a pharmaceutically acceptable carrier, adjuvant or vehicle.

The present invention also provides a pharmaceutically acceptable salt of a compound of the present invention.

The present invention also provides a composition comprising a pharmaceutically acceptable salt of the present invention and a pharmaceutically acceptable carrier, adjuvant or vehicle.

The present invention also provides a kit comprising a compound or composition of the present invention and instructions for use.

The present invention also provides a method for treating or ameliorating the effects of a neurodegenerative disorder in a subject in need thereof comprising administering to the subject an effective amount of a compound selected from the group consisting of formula (Ia)

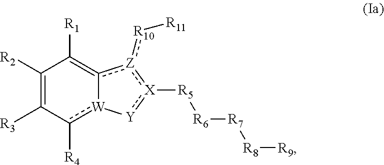

wherein a dashed line indicates the presence of an optional double bond,
wherein W, X, and Z are independently selected from the group consisting of C, N, S and O,
wherein Y is selected from the group consisting of C, N, Se, S and O, or another group VI atom,
wherein R₁, R₂, R₃, and R₄ are independently selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₅, R₆, R₇, R₈ and R₁₀ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₉ and R₁₁ are independently selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof, or an N-oxide, crystalline form, hydrate thereof, or a pharmaceutically acceptable salt thereof;
formula (II)

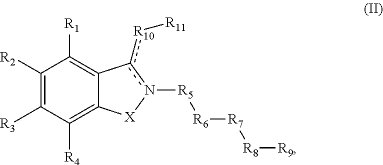

wherein a dashed line indicates the presence of an optional double bond,
wherein X is selected from the group consisting of S and Se,
wherein R₁, R₂, R₃, and R₄ are independently selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₅ is selected from the group consisting of C₁₋₄alkyl, C(O)NH, C(O), C(O)O, NH and O,
wherein R₆, is -(optionally substituted mono- or polycyclic group containing 3 to 20 carbon atoms and optionally 1 to 4 heteroatoms selected from O, N and S),
wherein R₇ and R₈ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₉ is selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₁₀ is selected from the group consisting of no atom and O,
wherein R₁₁ is selected from the group consisting of O and -(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
or an N-oxide, crystalline form, hydrate thereof, or a pharmaceutically acceptable salt thereof; and

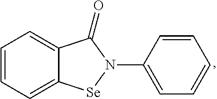 [SMILES: O=c1c2ccccc2[se]n1-c1ccccc1]

or an N-oxide, crystalline form, hydrate thereof, or a pharmaceutically acceptable salt thereof.

The present invention also provides a method for treating or ameliorating the effects of a neurodegenerative disorder in a subject in need thereof comprising administering to the subject an effective amount of a composition of the invention.

The present invention also provides a method for treating or ameliorating the effects of a condition associated with increased protein disulfide isomerase (PDI) activity in a subject in need thereof comprising administering to the subject an effective amount of a compound selected from the group consisting of formula (Ia)

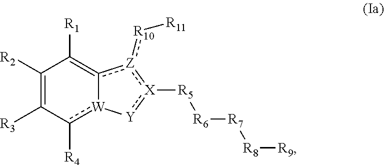

wherein a dashed line indicates the presence of an optional double bond,
wherein W, X, and Z are independently selected from the group consisting of C, N, S and O,
wherein Y is selected from the group consisting of C, N, Se, S and O, or another group VI atom,
wherein R₁, R₂, R₃, and R₄ are independently selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₅, R₆, R₇, R₈ and R₁₀ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₉ and R₁₁ are independently selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
or an N-oxide, crystalline form, hydrate thereof, or a pharmaceutically acceptable salt thereof;
formula (II)

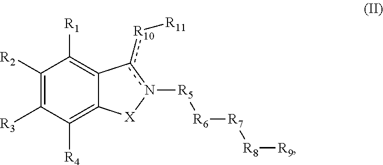

wherein a dashed line indicates the presence of an optional double bond,
wherein X is selected from the group consisting of S and Se,
wherein R₁, R₂, R₃, and R₄ are independently selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₅ is selected from the group consisting of C₁₋₄alkyl, C(O)NH, C(O), C(O)O, NH and O,
wherein R₆, is -(optionally substituted mono- or polycyclic group containing 3 to 20 carbon atoms and optionally 1 to 4 heteroatoms selected from O, N and S),
wherein R₇ and R₈ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₉ is selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₁₀ is selected from the group consisting of no atom and O,
wherein R₁₁ is selected from the group consisting of O and -(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
or an N-oxide, crystalline form, hydrate thereof, or a pharmaceutically acceptable salt thereof; and

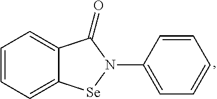 [SMILES: O=c1c2ccccc2[se]n1-c1ccccc1]

or an N-oxide, crystalline form, hydrate thereof, or a pharmaceutically acceptable salt thereof.

The present invention also provides a method for treating or ameliorating the effects of a condition associated with increased protein disulfide isomerase (PDI) activity in a subject in need thereof comprising administering to the subject an effective amount of a composition of the present invention.

The present invention also provides a method of modulating PDI activity in a cell comprising administering to the subject an effective amount of a compound selected from the group consisting of formula (Ia)

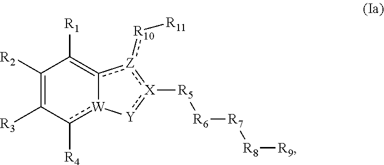

wherein a dashed line indicates the presence of an optional double bond,
wherein W, X, and Z are independently selected from the group consisting of C, N, S and O,
wherein Y is selected from the group consisting of C, N, Se, S and O, or another group VI atom,
wherein R₁, R₂, R₃, and R₄ are independently selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₅, R₆, R₇, R₈ and R₁₀ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₉ and R₁₁ are independently selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof, or an N-oxide, crystalline form, hydrate thereof, or a pharmaceutically acceptable salt thereof;
formula (II)

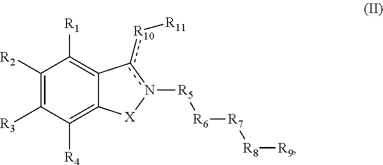

wherein a dashed line indicates the presence of an optional double bond,
wherein X is selected from the group consisting of S and Se,
wherein R₁, R₂, R₃, and R₄ are independently selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₅ is selected from the group consisting of C₁₋₄alkyl, C(O)NH, C(O), C(O)O, NH and O,
wherein R₆, is -(optionally substituted mono- or polycyclic group containing 3 to 20 carbon atoms and optionally 1 to 4 heteroatoms selected from O, N and S),
wherein R₇ and R₈ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₉ is selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₁₀ is selected from the group consisting of no atom and O,
wherein R₁₁ is selected from the group consisting of O and -(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof, or an N-oxide, crystalline form, hydrate thereof, or a pharmaceutically acceptable salt thereof; and

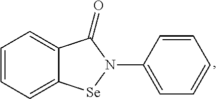 [SMILES: O=c1c2ccccc2[se]n1-c1ccccc1]

or an N-oxide, crystalline form, hydrate thereof, or a pharmaceutically acceptable salt thereof.

The present invention also provides a method of modulating PDI activity in a cell comprising administering to the subject an effective amount of a composition of the present invention.

The invention also provides a method for treating or ameliorating the effects of a neurodegenerative disorder in a subject in need thereof comprising administering to the subject an effective amount of a compound selected from the group consisting of:

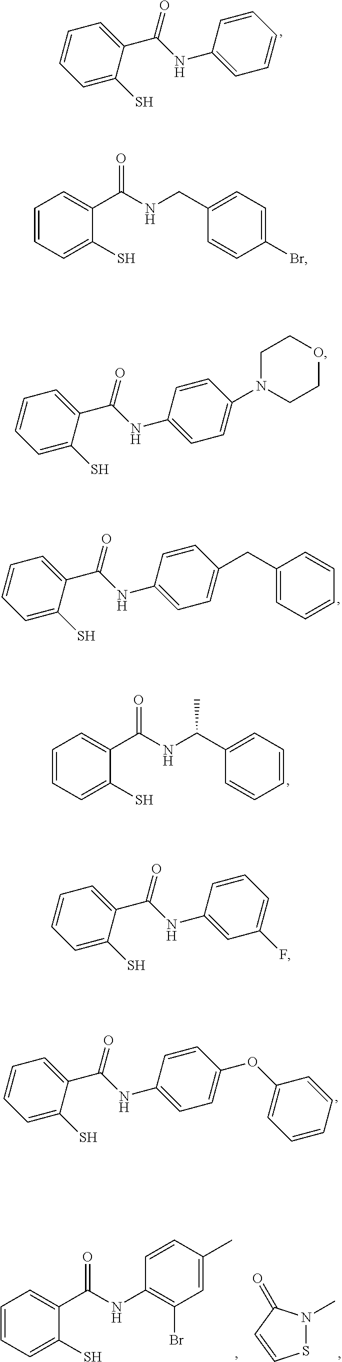

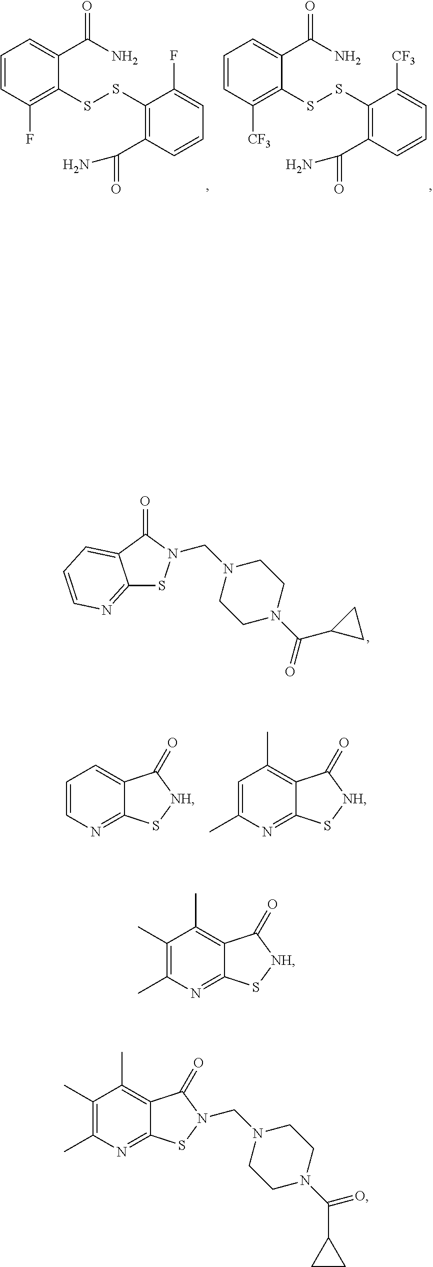

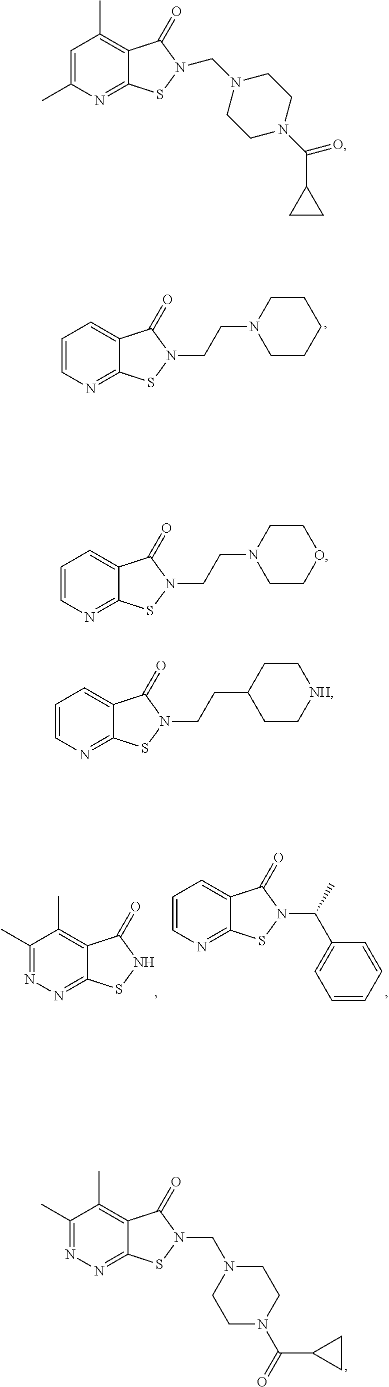

combinations thereof,
or an N-oxide, crystalline form, hydrate thereof, or a pharmaceutically acceptable salt thereof.

The present invention also provides a method for treating or ameliorating the effects of a condition associated with increased protein disulfide isomerase (PDI) activity in a subject in need thereof comprising administering to the subject an effective amount of a compound selected from the group consisting of:

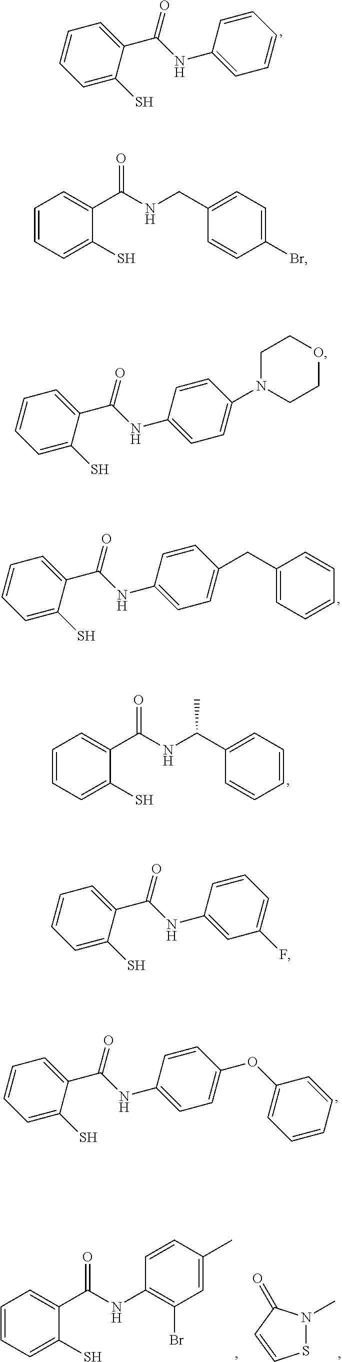

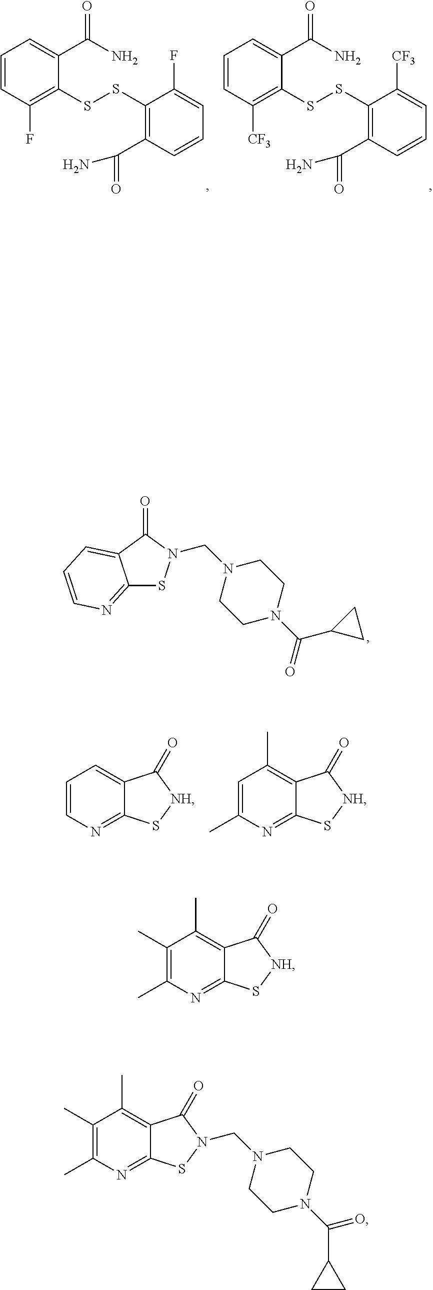

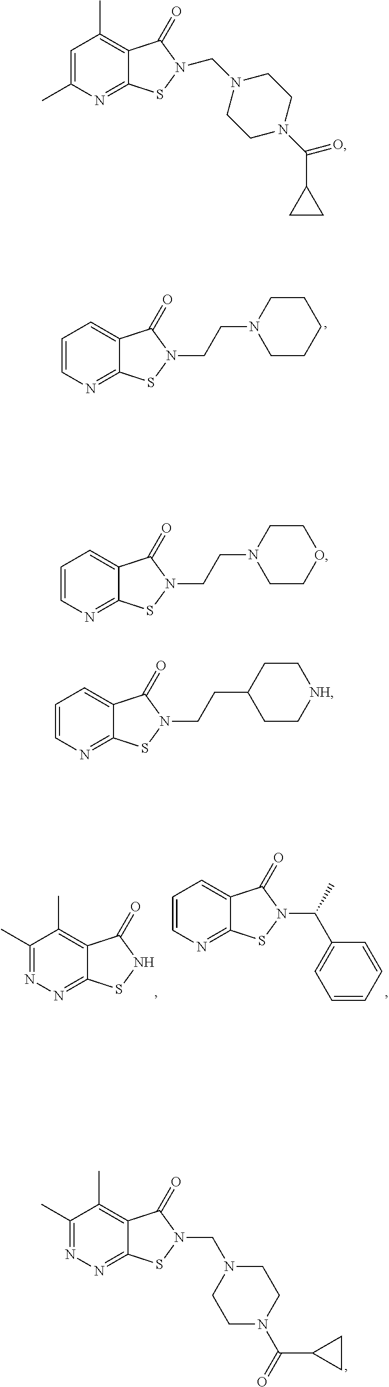

combinations thereof, or an N-oxide, crystalline form, hydrate thereof, or a pharmaceutically acceptable salt thereof.

The present invention also provides a method of modulating PDI activity in a cell comprising contacting the cell with an effective amount of a compound selected from the group consisting of:

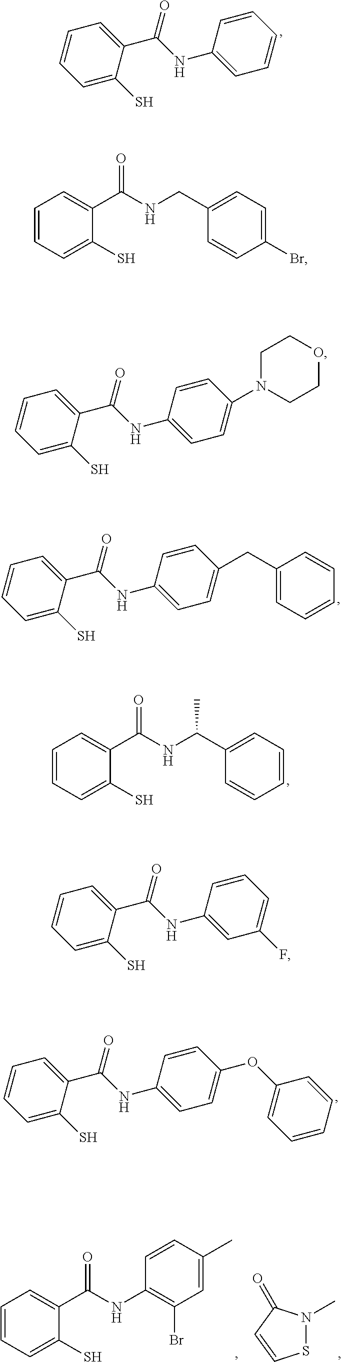

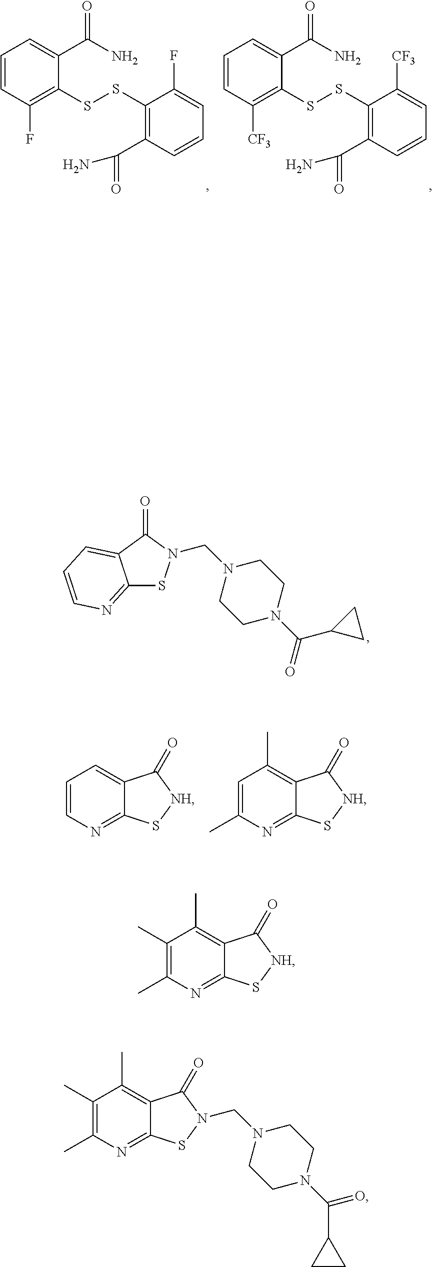

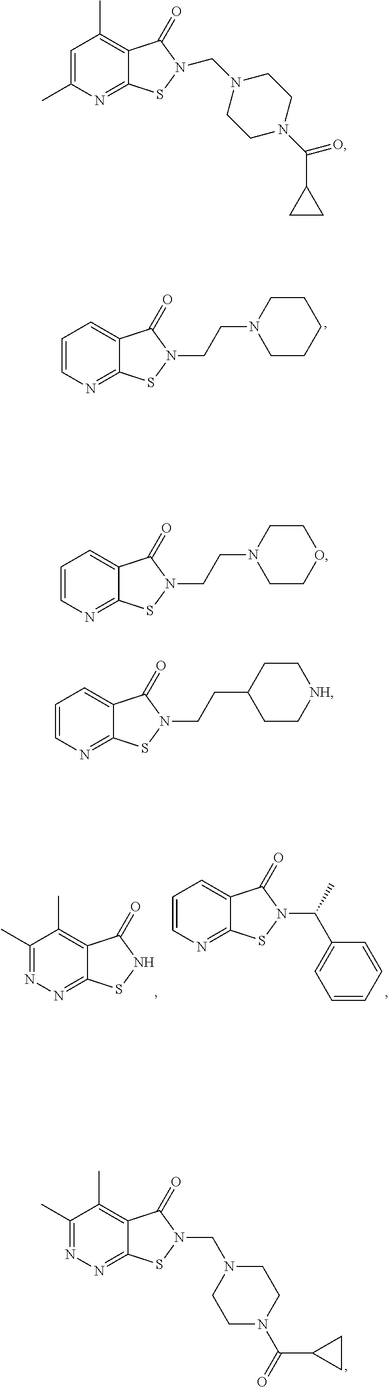

combinations thereof, or an N-oxide, crystalline form, hydrate thereof, or a pharmaceutically acceptable salt thereof.

The present invention also provides a compound selected from the group consisting of:

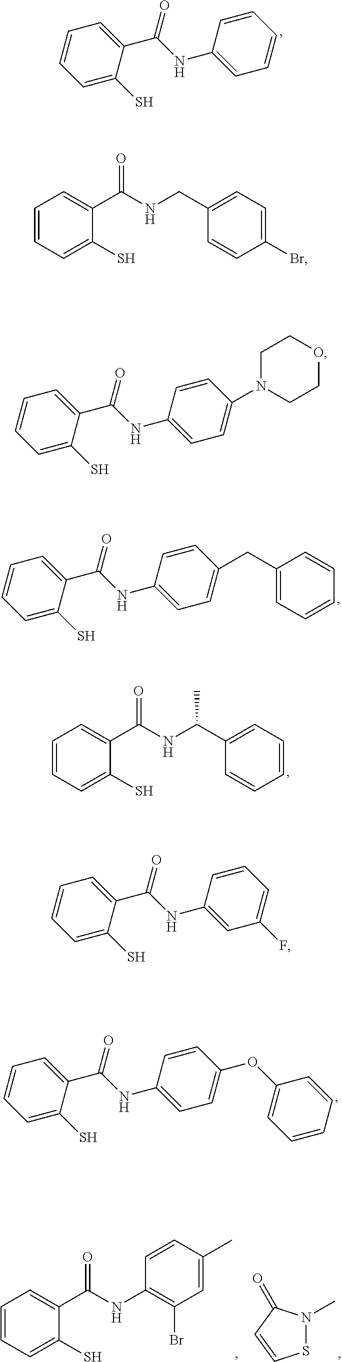

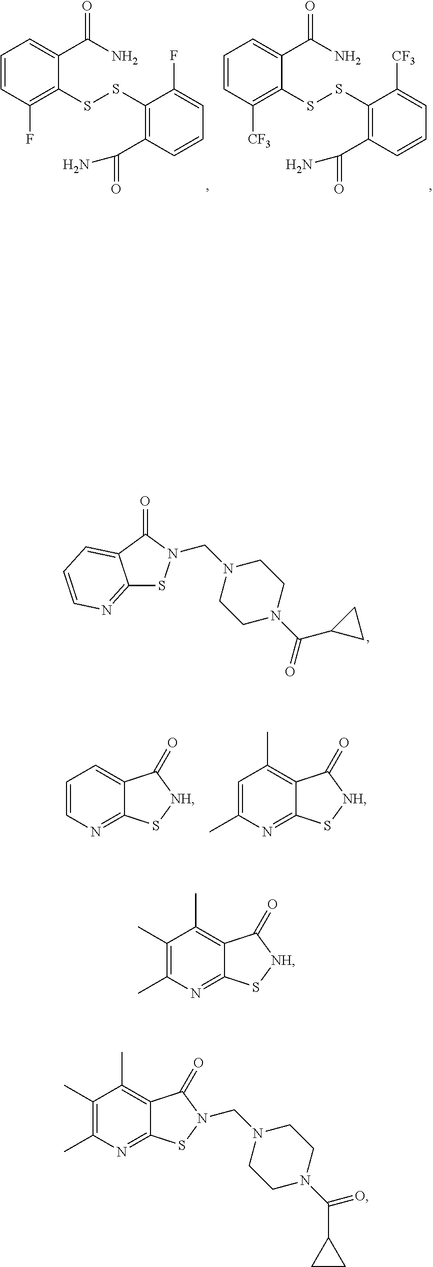

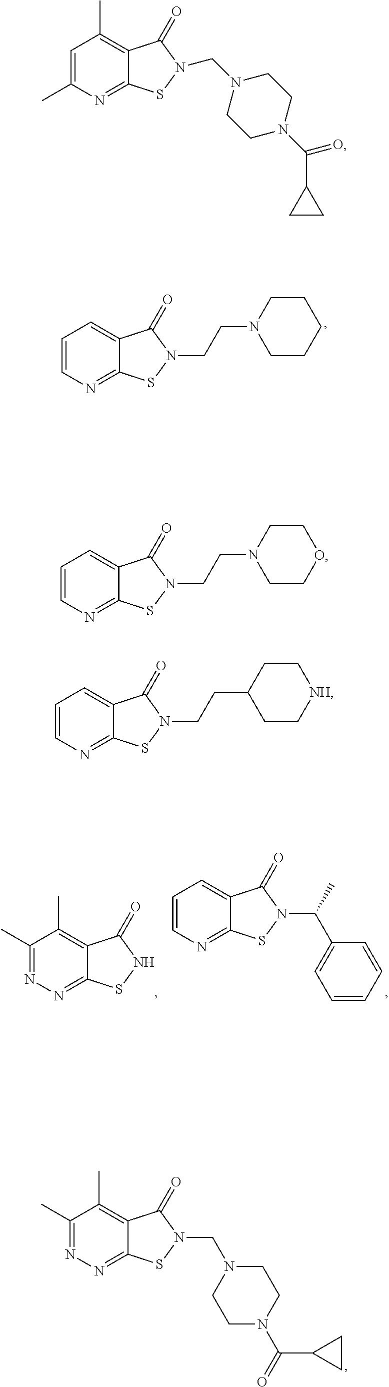

combinations thereof, or an N-oxide, crystalline form, hydrate thereof, or a pharmaceutically acceptable salt thereof. The present invention further provides compositions that include any of the foregoing compounds individually or in any combination, and a pharmaceutically acceptable carrier, adjuvant or vehicle.

### BRIEF DESCRIPTION OF THE DRAWINGS

The application file contains at least one drawing executed in color. Copies of this patent application publication with color drawing(s) will be provided by the Office upon request and payment of the necessary fee.

The following drawings form part of the present specification and are included to further demonstrate certain aspects of the present invention. The invention may be better understood by reference to one or more of these drawings in combination with the detailed description of specific embodiments presented herein.

FIG. 1 shows the ¹H NMR spectrum of LOC14. Re-synthesized LOC14 structure was validated by NMR.

FIG. 2A-FIG. 2B shows a high throughput screen identifying neuroprotective PDI inhibitors. FIG. 2A are dose-response curves of three top hits that rescued PC12 cells from mHTT^{Q103} induced cell death as measured by Alamar blue fluorescence after 48 hours treatment. Data from cells induced to express mHTT^{Q103} (blue) and cells not expressing mHTT^{Q103} (red) are plotted as mean percent of DMSO treated uninduced cells±SD. Experiments were performed in triplicate. FIG. 2B shows the secondary screen of the top three hits (75 μM) for their ability to inhibit the enzymatic activity of PDIa (5 μM) in an insulin aggregation assay. Experiments were performed in duplicate with data plotted as mean±SEM.

FIG. 3A-FIG. 3C shows evaluation of hits from a high throughput screen. FIG. 3A-FIG. 3B are dose-response curves of five additional HTS hits that rescued PC12 cells from mHTT^{Q103} induced cell death as measured by Alamar blue fluorescence after 48 hours treatment. Data from cells induced to express mHTT^{Q103} (blue) and cells not expressing mHTT^{Q103} (red) are plotted as mean percent of DMSO treated uninduced cells±SD. Experiments were performed in triplicate. FIG. 3C shows the five hits at 75 μM evaluated in counter-screen for their ability to inhibit the enzymatic activity of PDIa (5 μM) in the insulin aggregation assay. Experiments were performed in duplicate with data plotted as mean±SEM.

FIG. 4A-FIG. 4C shows that sulfur in LOC14 is important for tight binding to PDIa. FIG. 4A shows calorimetric titration of 400 μM LOC14 into 40 μM PDIa and FIG. 4B shows 400 μM Oxy-LOC14 into 40 μM PDIa. Upper panels show the raw data of the heat released; lower panels show the binding isotherm of the reaction. Data are fit to one-site binding model after subtracting the heat released from titrating the compound alone into buffer. One of three representative experiments is shown. FIG. 4C is a summary graph of thermodynamic parameters for binding. Data are plotted as mean±SD (n=3).

FIG. 5A-FIG. 5C shows that LOC14 binds reversibly to PDIa. FIG. 5A shows fluorescence emission spectra of LOC14, PDIa, or PDIa-LOC14 complex before size-exclusion buffer dialysis. FIGS. 5B and 5C show the fluorescence emission spectra after size-exclusion buffer dialysis. For the dialysis, 10 kDa size exclusion filter spin columns were used. FIG. 5B shows fluorescence emission spectra of fractions larger than 10 kDa that were retained in the dialysis chamber. FIG. 5C shows fluorescence emission spectra of fractions larger than 10 kDa that were collected from the flow-through of dialysis. All samples were excited at 280 nm and emission spectra recorded from 315 nm-550 nm.

FIG. 6 shows representative strip plots of 3D ¹H-¹⁵N-NOESY-HSQC (red) and 3D ¹H-¹⁵N-TOCSY-HSQC (blue) for residues L62-A67 in the reduced a domain of PDI A1. The peaks represent NOE signal between the amide hydrogen to any hydrogen within its own spin system (for ¹⁵N-TOCSY-HSQC) or to any hydrogen within 5 Å proximity in space (¹⁵N-NOESY-HSQC). ¹⁵N-TOCSY-HSQC spectrum helped identify the spin system and the NOEs corresponding to the amide, alpha, or beta protons within that spin system (labeled). Those assigned NOEs were then transferred to ¹⁵N-NOESY-HSQC (green circles) to be used for sequential assignments (dotted black path).

FIG. 7A-FIG. 7D shows chemical shift changes in PDIa upon binding LOC14. FIG. 7A shows superimposed HSQC spectra of PDIa alone (black) and PDIa treated with 1 mol. equiv. of LOC14 (green). Resonances with largest chemical shifts (mean shift change+1×SD) are labeled in red. FIG. 7B is a zoom-in on the most shifted peaks. The abrupt progression of PDIa peaks as LOC14 is titrated at 0 (black), 0.25 (purple), 0.5 (blue), 1 (green), 2.5 (orange) and 10 (red) fold molar excess. After the saturation point with 1 mol. equiv. of LOC14 (green) no further shifts are observed. FIG. 7C shows a graph of chemical shift differences (Δδ_{NH}) for each residue in the PDIa sequence upon 1:1 PDIa:LOC14 binding. Weighted mean of ¹H and ¹⁵N chemical shift changes is plotted as a red line; the mean shift change+1×SD is plotted as a dotted blue line. FIG. 7D shows chemical shift perturbations used to map the LOC14 binding site onto the molecular surface of reduced PDIa (PDB: 4EKZ).

FIG. 8 shows that LOC14 binding to PDIa induces an oxidized conformation in the protein. The ¹H-¹⁵N HSQC spectra of 50 μM oxidized PDIa alone (black) and 100 μM reduced PDIa treated with 100 μM LOC14 (red) are superimposed. Residue R80 (green circle) is the only peak that is different between the two spectra.

FIG. 9A-9G shows that LOC14 has a different mode of binding to PDIa than irreversible inhibitor 16F16. FIG. 9A shows that LC/MS had 95% sequence coverage of PDIa (red bold) (SEQ ID NO: 1) when treated with 16F16. FIG. 9B-FIG. 9C shows the predicted and observed fragment ion (ms/ms) mass spectrum and table of the YLLVEFYAPWCGHCK (SEQ ID NO: 2) peptide from the trypsin digested PDIa (100 μM) treated with 16F16 (500 μM) overnight. Ion score was 58, precursor RMS error was 3 ppm, and product RMS error was 5 ppm. The predicted and observed y-ion masses are different because of a 284.1161 m/z (monoisotopic) modification at each cysteine. Blue are observed y-ion masses. FIG. 9D is a schematic showing the modification at each cysteine upon 16F16 binding to PDIa, which causes a 284.1161 mass increase. FIG. 9E shows ITC titration of 400 μM LOC14 against 40 μM PDIa that has been pre-treated overnight with irreversible inhibitor 16F16 (200 μM). Upper panel shows the raw data of the heat released; lower panel shows the binding isotherm of the reaction, fit to one-site binding model after subtracting the heat released from titrating LOC14 into buffer with 16F16. One of three representative experiments is shown. FIG. 9F is a summary graph of thermodynamic parameters for binding. Data are plotted as mean±SD (n=3). FIG. 9G shows superimposed HSQC spectra of 50 μM PDIa alone (black), 100 μM PDIa treated with 100 μM LOC14 (green), and 50 μM PDIa treated with 250 μM 16F16 (purple). The arrows indicate the direction of the shift.

FIG. 10 shows that LOC14 rescues striatal medium spiny neurons (MSNs) from mutant huntingtin-induced neurodegeneration in brain slice explants. Rat corticostriatal brain slice explants co-transfected with YFP and the first exon of mutant HTT gene (mHTT-Q73) were treated with LOC14, a positive control compound mixture of 50 μM KW-6002 and 30 μM of SP600125, or DMSO only for 4 days. Data are plotted as mean±SEM from one of two representative experiments. *Significant by ANOVA followed by Dunnett's post hoc comparison test at p<0.05

FIG. 11 shows a possible mechanism for LOC14 modulation of PDI activity. 36 and 39 correspond to the residue number of the two cysteines in the active site. The residue numbering is based on the sequence of the mature PDI protein.

FIG. 12A-FIG. 12B shows an overview of the two screens used to identify neuroprotective PDI inhibitors.

FIG. 13-FIG. 13B shows recovery of enzymatic activity of PDIa and demonstrates that LOC14 reversibly binds to PDIa. PDIa (500 μM) was incubated with either (FIG. 13A) irreversible inhibitor 16F16 (750 μM) or (FIG. 13B) LOC14 (750 μM) for three hours at room temperature and then diluted 100-fold into assay buffer and analyzed for its ability to inhibit the enzymatic insulin aggregation. Diluted complexes were compared to samples containing 5 μM PDIa only (red), or 5 μM PDIa with either 7.5 μM or 750 μM compound LOC14 or 16F16. Experiments were performed in triplicate with data plotted as mean±SEM.

FIG. 14 shows that LOC14 binding to PDIa induces an oxidized conformation in the protein. Calorimetric titration of 400 μM LOC14 into 40 μM oxidized PDIa: the upper panel shows the raw data of the heat released; the lower panel shows the binding isotherm of the reaction. Data are fit to one-site binding model after subtracting the heat released from titrating the compound alone into buffer.

FIG. 15A-FIG. 15B shows that LOC14 is metabolically stable compound. (FIG. 15A) LOC14 is stable in mouse liver microsomes. 7-Ethoxycoumarin, a substrate of cytochrome P450 enzymes, was used as a control. (FIG. 15B) LOC14 is stable in mouse plasma. Enalapril, which undergoes degradation in plasma, was used as a control compound.

FIG. 16 shows that LOC14 rescues cortical neurons from Tau4R-induced neurodegeneration in brain slice explants. Rat corticostriatal brain slice explants co-transfected with YFP and the tau isoform with 4 tubulin-binding repeats (Tau4R) were treated with LOC14, an analog of LOC14 “BIT fragment” (1,2,Benzisothiazol-3-one), or DMSO only for 4 days. Data are plotted as mean±SEM from one of five representative experiments. *Significant by ANOVA followed by Dunnett's post hoc comparison test at p<0.05.

FIG. 17A-FIG. 17B shows that LOC14 can traverse the BBB in vivo. LOC14 administered via (FIG. 17A) intravenous or (FIG. 17B) oral route to wild-type C57BL/6j mice at 20 mg/kg. The concentration of compound in the brain tissue and plasma is shown for each individual mouse (represented by dots). The horizontal lines represent the mean±SD (n=3).

### DETAILED DESCRIPTION OF THE INVENTION

One embodiment of the present invention is a method for treating or ameliorating the effects of a neurodegenerative disorder in a subject in need thereof comprising administering to the subject an effective amount of a compound selected from the group consisting of:

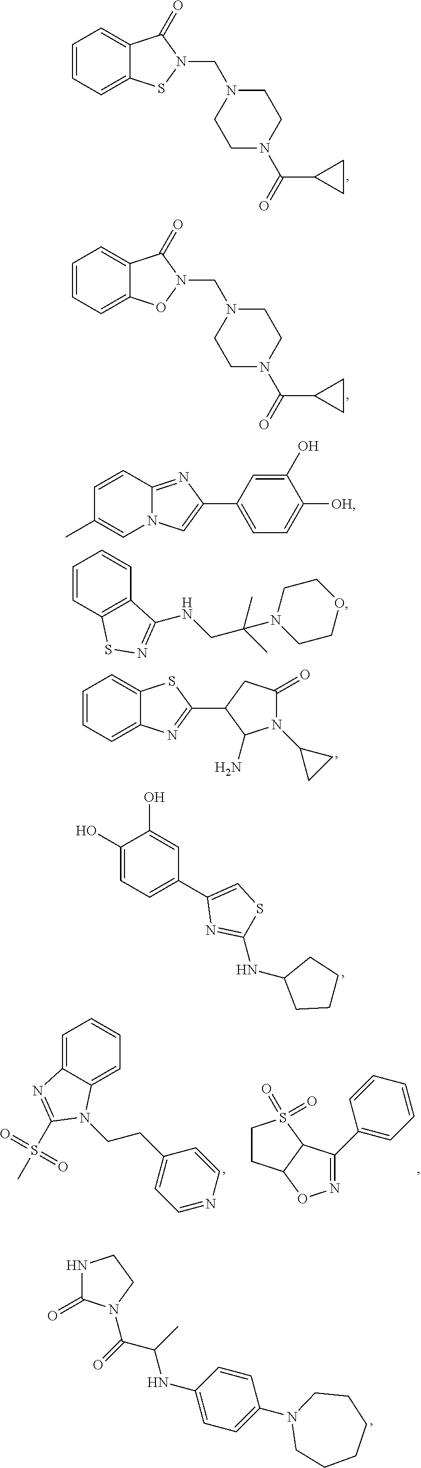

combinations thereof,
or an N-oxide, crystalline form, hydrate thereof, or a pharmaceutically acceptable salt thereof.

As used herein, an “N-oxide” means a compound containing an N—O bond with three additional hydrogen and/or side chains attached to N, so that there is a positive charge on the nitrogen. The N-oxides of compounds of the present invention may be synthesized by simple oxidation procedures well known to those skilled in the art. For example, the oxidation procedure described by P. Brougham et al. (Synthesis, 1015-1017, 1987), allows the two nitrogen of a piperazine ring to be differentiated, enabling both the N-oxides and N,N′-dioxide to be obtained. Other oxidation procedures are disclosed in, e.g., U.S. Patent Publication No. 20070275977; S. L. Jain, J. K. Joseph, B. Sain, Synlett, 2006, 2661-2663; A. McKillop, D. Kemp, Tetrahedron, 1989, 45, 3299-3306; R. S. Varma, K. P. Naicker, Org. Lett., 1999, 1, 189-191; and N. K. Jana, J. G. Verkade, Org. Lett., 2003, 5, 3787-3790. Thus, the present invention includes these and other well known procedures for making N-oxides, so long as the end product is sufficiently effective as set forth in more detail below.

The term “crystalline form”, as used herein, refers to the crystal structure of a compound. A compound may exist in one or more crystalline forms, which may have different structural, physical, pharmacological, or chemical characteristics. Different crystalline forms may be obtained using variations in nucleation, growth kinetics, agglomeration, and breakage. Nucleation results when the phase-transition energy barrier is overcome, thereby allowing a particle to form from a supersaturated solution. Crystal growth is the enlargement of crystal particles caused by deposition of the chemical compound on an existing surface of the crystal. The relative rate of nucleation and growth determine the size distribution of the crystals that are formed. The thermodynamic driving force for both nucleation and growth is supersaturation, which is defined as the deviation from thermodynamic equilibrium. Agglomeration is the formation of larger particles through two or more particles (e.g., crystals) sticking together and forming a larger crystalline structure.

As used herein, a “hydrate” means a compound that contains water molecules in a definite ratio and in which water forms an integral part of the crystalline structure of the compound. Methods of making hydrates are known in the art. For example, some substances spontaneously absorb water from the air to form hydrates. Others may form hydrates upon contact with water. In most cases, however, hydrates are made by changes in temperature or pressure. Additionally, the compounds of the present invention as well as their salts may contain, e.g., when isolated in crystalline form, varying amounts of solvents, such as water. Included within the scope of the invention are, therefore, all hydrates of the compounds and all hydrates of salts of the compounds of the present invention, so long as such hydrates are sufficiently effective as set forth in more detail below.

In one aspect of this embodiment the neurodegenerative disease is selected from the group consisting of Alzheimer's disease, Parkinson's disease, amyotrophic lateral sclerosis (ALS), Friedreich's ataxia, multiple sclerosis, Huntington's Disease, transmissible spongiform encephalopathy, Charcot-Marie-Tooth disease, dementia with Lewy bodies, corticobasal degeneration, progressive supranuclear palsy, and hereditary spastic paraparesis. In a preferred embodiment, the neurodegenerative disease is Huntington's Disease.

In one aspect of this embodiment the compound is

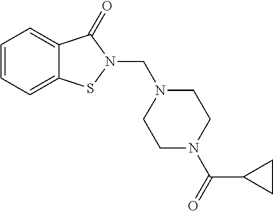 [SMILES: O=C(C1CC1)N1CCN(Cn2sc3ccccc3c2=O)CC1]

or an N-oxide, crystalline form, hydrate thereof, or a pharmaceutically acceptable salt thereof.

In another aspect of this embodiment the subject is a mammal. In some aspects of this embodiment the mammal is selected from the group consisting of humans, veterinary animals, and agricultural animals. In another aspect of this embodiment the subject is a human.

In one aspect of this embodiment the method further comprises co-administering to the subject an effective amount of one or more additional therapeutic agents. Preferably, the one or more additional therapeutic agents are selected from the group consisting of 5-hydroxytryptophan, Activase, AFQ056 (Novartis), Aggrastat, Albendazole, alpha-lipoic acid/L-acetyl carnitine, Alteplase, Amantadine (Symmetrel), amlodipine, Ancrod, Apomorphine (Apokyn), Arimoclomol, Arixtra, Armodafinil, Ascorbic acid, Ascriptin, Aspirin, atenolol, Avonex, baclofen (Lioresal), Banzel, Benztropine (Cogentin), Betaseron, BGG492 (Novartis Corp.), Botulinum toxin, Bufferin, Carbatrol®, Carbidopa/levodopa immediate-release (Sinemet), Carbidopa/levodopa oral disintegrating (Parcopa), Carbidopa/levodopa/Entacapone (Stalevo), CERE-110: Adeno-Associated Virus Delivery of NGF (Ceregene), cerebrolysin, CinnoVex, citalopram, citicoline, Clobazam, Clonazepam, Clopidogrel, clozapine (Clozaril), Coenzyme Q, Creatine, dabigatran, dalteparin, Dapsone, Davunetide, Deferiprone, Depakene®, Depakote ER®, Depakote®, Desmoteplase, Diastat, Diazepam, Digoxin, Dilantin®, Dimebon, dipyridamole, divalproex (Depakote), Donepezil (Aricept), EGb 761, Eldepryl, ELND002 (Elan Pharmaceuticals), Enalapril, enoxaparin, Entacapone (Comtan), epoetin alfa, Eptifibatide, Erythropoietin, Escitalopram, Eslicarbazepine acetate, Esmolol, Ethosuximide, Ethyl-EPA (Miraxion™), Exenatide, Extavia, Ezogabine, Felbamate, Felbatol®, Fingolimod (Gilenya), fluoxetine (Prozac), fondaparinux, Fragmin, Frisium, Gabapentin, Gabitril®, Galantamine, Glatiramer (Copaxone), haloperidol (Haldol), Heparin, human chorionic gonadotropin (hCG), Idebenone, Inovelon®, insulin, Interferon beta 1a, Interferon beta 1b, ioflupane 1231 (DATSCAN®), IPX066 (Impax Laboratories Inc.), JNJ-26489112 (Johnson and Johnson), Keppra®, Klonopin, Lacosamide, L-Alpha glycerylphosphorylcholine, Lamictal®, Lamotrigine, Levetiracetam, liraglutide, Lisinopril, Lithium carbonate, Lopressor, Lorazepam, losartan, Lovenox, Lu AA24493, Luminal, LY450139 (Eli Lilly), Lyrica, Masitinib, Mecobalamin, Memantine, methylprednisolone, metoprolol tartrate, Minitran, Minocycline, mirtazapine, Mitoxantrone (Novantrone), Mysoline®, Natalizumab (Tysabri), Neurontin®, Niacinamide, Nitro-Bid, Nitro-Dur, nitroglycerin, Nitrolingual, Nitromist, Nitrostat, Nitro-Time, Norepinephrine (NOR), Carbamazepine, octreotide, Onfi®, Oxcarbazepine, Oxybutinin chloride, PF-04360365 (Pfizer), Phenobarbital, Phenytek®, Phenytoin, piclozotan, Pioglitazone, Plavix, Potiga, Pramipexole (Mirapex), pramlintide, Prednisone, Primidone, Prinivil, probenecid, Propranolol, PRX-00023 (EPIX Pharmaceuticals Inc.), PXT3003, Quinacrine, Ramelteon, Rasagiline (Azilect), Rebif, ReciGen, remacemide, Resveratrol, Retavase, reteplase, riluzole (Rilutek), Rivastigmine (Exelon), Ropinirole (Requip), Rotigotine (Neupro), Rufinamide, Sabril, safinamide (EMD Serono), Salagen, Sarafem, Selegiline (1-deprenyl, Eldepryl), SEN0014196 (Siena Biotech), sertraline (Zoloft), Simvastatin, Sodium Nitroprussiate (NPS), sodium phenylbutyrate, Stanback Headache Powder, Tacrine (Cognex), Tamoxifen, tauroursodeoxycholic acid (TUDCA), Tegretol®, Tenecteplase, Tenormin, Tetrabenazine (Xenazine), THR-18 (Thrombotech Ltd.), Tiagabine, Tideglusib, tirofiban, tissue plasminogen activator (tPA), tizanidine (Zanaflex), TNKase, Tolcapone (Tasmar), Tolterodine, Topamax®, Topiramate, Trihexyphenidyl (formerly Artane), Trileptal®, ursodiol, Valproic Acid, valsartan, Varenicline (Pfizer), Vimpat, Vitamin E, Warfarin, Zarontin®, Zestril, Zonegran®, Zonisamide, Zydis selegiline HCL Oral disintegrating (Zelapar), and combinations thereof.

Another embodiment of the present invention is a method for treating or ameliorating the effects of a condition associated with increased protein disulfide isomerase (PDI) activity in a subject in need thereof comprising administering to the subject an effective amount of a compound selected from the group consisting of:

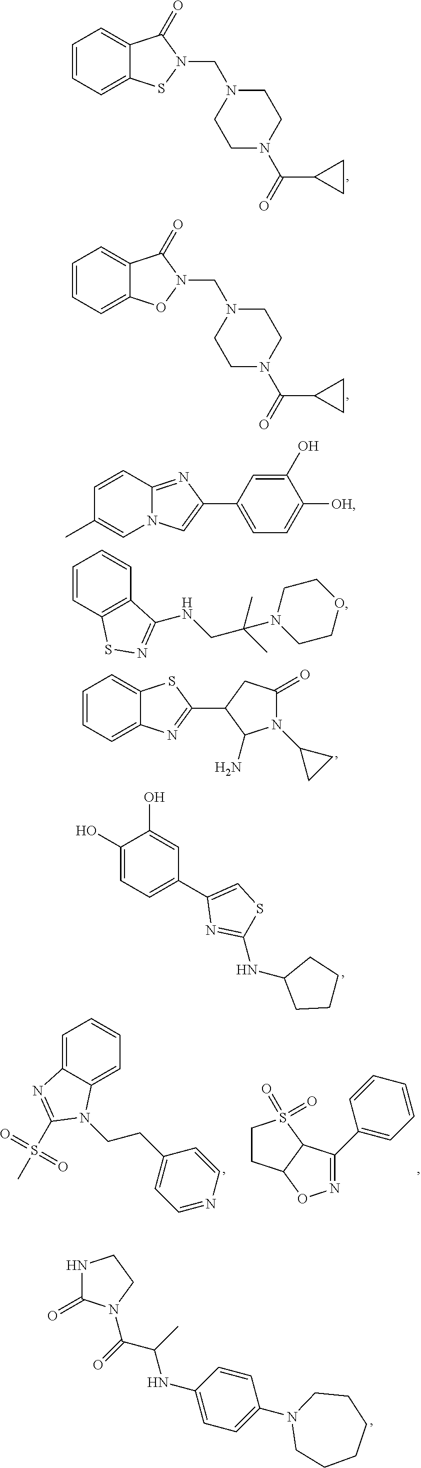

combinations thereof,
or an N-oxide, crystalline form, hydrate thereof, or a pharmaceutically acceptable salt thereof.

In one aspect of this embodiment the compound is

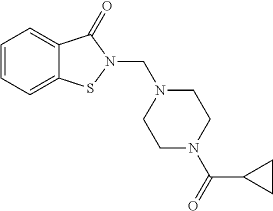 [SMILES: O=C(C1CC1)N1CCN(Cn2sc3ccccc3c2=O)CC1]

or an N-oxide, crystalline form, hydrate thereof, or a pharmaceutically acceptable salt thereof.

In another aspect of this embodiment the subject is a mammal. In some aspects of this embodiment the mammal is selected from the group consisting of humans, veterinary animals, and agricultural animals. In another aspect of this embodiment the subject is a human.

In one aspect of this embodiment the condition is a protein folding disorder. In another aspect of this embodiment the condition is cancer. In yet another aspect of this embodiment the condition is HIV. In yet another aspect of this embodiment the condition is a blood clot.

Another embodiment of the present invention is a method of modulating PDI activity in a cell comprising contacting the cell with an effective amount of a compound selected from the group consisting of:

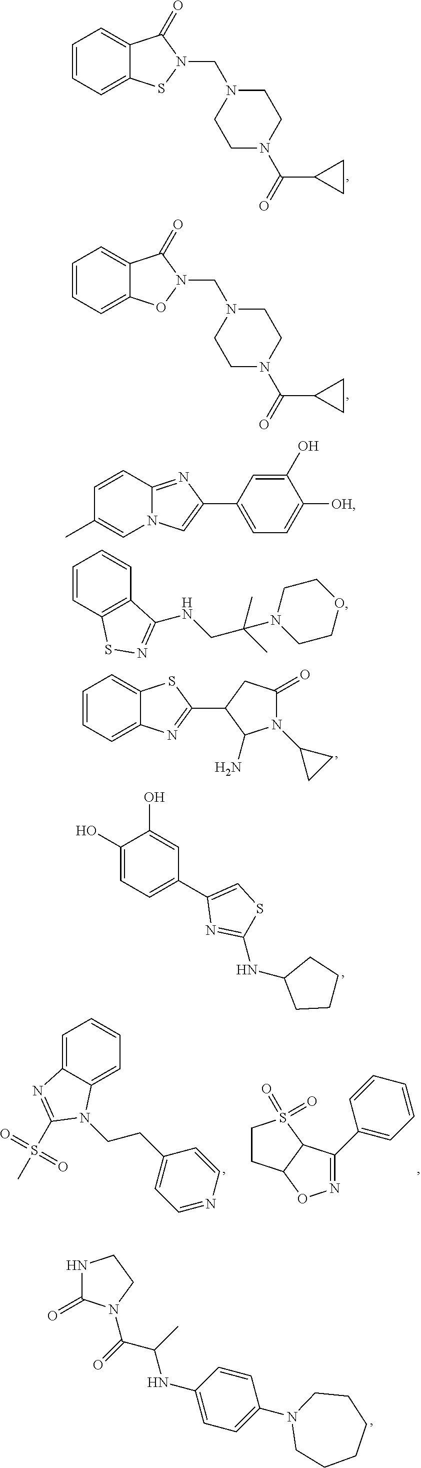

combinations thereof,
or an N-oxide, crystalline form, hydrate thereof, or a pharmaceutically acceptable salt thereof.

In one aspect of this embodiment the compound is

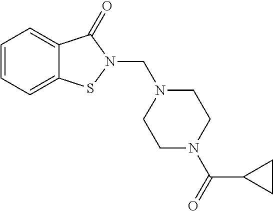 [SMILES: O=C(C1CC1)N1CCN(Cn2sc3ccccc3c2=O)CC1]

or an N-oxide, crystalline form, hydrate thereof, or a pharmaceutically acceptable salt thereof.

Another embodiment of the present invention is a method for treating or ameliorating the effects of a neurodegenerative disorder in a subject in need thereof comprising administering to the subject an effective amount of a compound selected from the group consisting of formula (I)

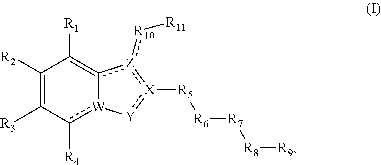

formula (II)

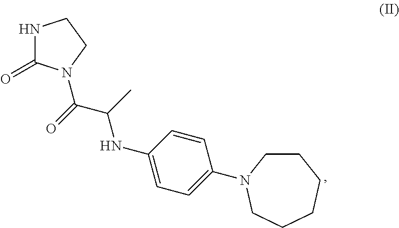 [SMILES: CC(Nc1ccc(N2CCCCCC2)cc1)C(=O)N1CCNC1=O]

formula (III)

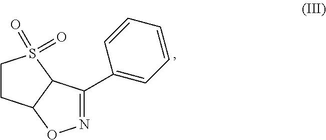 [SMILES: O=S1(=O)CCC2ON=C(c3ccccc3)C21]

and formula (IV):

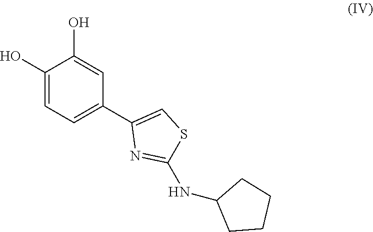 [SMILES: Oc1ccc(-c2csc(NC3CCCC3)n2)cc1O]

wherein a dashed line indicates the presence of an optional double bond,
wherein W, X, Y and Z are independently selected from the group consisting of C, N, S and O,
wherein R₁, R₂, R₃, and R₄ are independently selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₅, R₆, R₇, R₈ and R₁₀ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₉ and R₁₁ are independently selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
or an N-oxide, crystalline form, hydrate thereof, or a pharmaceutically acceptable salt thereof.

The term “aliphatic”, as used herein, refers to a group composed of carbon and hydrogen atoms that do not contain aromatic rings. Accordingly, aliphatic groups include alkyl, alkenyl, alkynyl, and carbocyclyl groups. Additionally, unless otherwise indicated, the term “aliphatic” is intended to include both “unsubstituted aliphatics” and “substituted aliphatics”, the latter of which refers to aliphatic moieties having substituents replacing a hydrogen on one or more carbons of the aliphatic group. Such substituents can include, for example, a halogen, a deuterium, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, an aromatic, or heteroaromatic moiety.

The term “alkyl” refers to the radical of saturated aliphatic groups that does not have a ring structure, including straight-chain alkyl groups, and branched-chain alkyl groups. In certain embodiments, a straight chain or branched chain alkyl has 6 or fewer carbon atoms in its backbone (e.g., C₁-C₆ for straight chains, C₃-C₆ for branched chains). Such substituents include all those contemplated for aliphatic groups, except where stability is prohibitive.

Moreover, unless otherwise indicated, the term “alkyl” as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Indeed, unless otherwise indicated, all groups recited herein are intended to include both substituted and unsubstituted options.

The term “alkenyl”, as used herein, refers to an aliphatic group containing at least one double bond and unless otherwise indicated, is intended to include both “unsubstituted alkenyls” and “substituted alkenyls”, the latter of which refers to alkenyl moieties having substituents replacing a hydrogen on one or more carbons of the alkenyl group. Such substituents include all those contemplated for aliphatic groups, as discussed below, except where stability is prohibitive. For example, substitution of alkenyl groups by one or more alkyl, carbocyclyl, aryl, heterocyclyl, or heteroaryl groups is contemplated.

The term “C_{x-y}” when used in conjunction with a chemical moiety, such as, alkyl and cycloalkyl, is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-tirfluoroethyl, etc.

The terms “amine” and “amino” are art-recognized and refer to both unsubstituted and substituted amines and salts thereof, e.g., a moiety that can be represented by

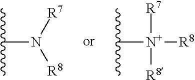

wherein R⁷, R⁸, and R^{8′} each independently represent a hydrogen or a hydrocarbyl group, or R⁷ and R⁸ taken together with the N atom to which they are attached complete a heterocycle having from 4 to 8 atoms in the ring structure. The term “primary” amine means only one of R⁷ and R⁸ or one of R⁷, R⁸, and R^{8′} is a hydrocarbyl group. Secondary amines have two hydrocarbyl groups bound to N. In tertiary amines, all three groups, R⁷, R⁸, and R^{8′}, are replaced by hydrocarbyl groups.

The term “aryl” as used herein includes substituted or unsubstituted single-ring aromatic groups in which each atom of the ring is carbon. Preferably the ring is a 3- to 8-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “alkyl-aryl” refers to an alkyl group substituted with at least one aryl group.

The terms “halo” and “halogen” are used interchangeably herein and mean halogen and include chloro, fluoro, bromo, and iodo.

The term “heterocycle” refers to substituted or unsubstituted non-aromatic ring structures, preferably 3- to 8-membered rings, whose ring structures include at least one heteroatom, preferably one to four heteroatoms, more preferably one or two heteroatoms. The term “heterocycle” also includes polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocycle groups include, for example, piperidine, piperazine, pyrrolidine, morpholine, lactones, lactams, and the like.

The term “heteroatom” as used herein means an atom of any element other than carbon or hydrogen. Preferred heteroatoms are nitrogen, oxygen, and sulfur; more preferably, nitrogen and oxygen.

The term “substituted” refers to moieties having substituents replacing a hydrogen on one or more carbons of the backbone. It will be understood that “substitution” or “substituted with” includes the implicit proviso that such substitution is in accordance with the permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

As set forth previously, unless specifically stated as “unsubstituted,” references to chemical moieties herein are understood to include substituted variants. For example, reference to an “aryl” group or moiety implicitly includes both substituted and unsubstituted variants.

In one aspect of this embodiment the neurodegenerative disease is selected from the group consisting of Alzheimer's disease, Parkinson's disease, amyotrophic lateral sclerosis (ALS), Friedreich's ataxia, multiple sclerosis, Huntington's Disease, transmissible spongiform encephalopathy, Charcot-Marie-Tooth disease, dementia with Lewy bodies, corticobasal degeneration, progressive supranuclear palsy, and hereditary spastic paraparesis. In a preferred embodiment, the neurodegenerative disease is Huntington's Disease.

In one aspect of this embodiment the compound is

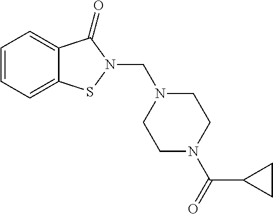 [SMILES: O=C(C1CC1)N1CCN(Cn2sc3ccccc3c2=O)CC1]

or an N-oxide, crystalline form, hydrate thereof, or a pharmaceutically acceptable salt thereof.

In another aspect of this embodiment the subject is a mammal. In some aspects of this embodiment the mammal is selected from the group consisting of humans, veterinary animals, and agricultural animals. In another aspect of this embodiment the subject is a human.

In one aspect of this embodiment the method further comprises co-administering to the subject an effective amount of one or more additional therapeutic agents. Preferably, the one or more additional therapeutic agents are selected from the group consisting of 5-hydroxytryptophan, Activase, AFQ056 (Novartis), Aggrastat, Albendazole, alpha-lipoic acid/L-acetyl carnitine, Alteplase, Amantadine (Symmetrel), amlodipine, Ancrod, Apomorphine (Apokyn), Arimoclomol, Arixtra, Armodafinil, Ascorbic acid, Ascriptin, Aspirin, atenolol, Avonex, baclofen (Lioresal), Banzel, Benztropine (Cogentin), Betaseron, BGG492 (Novartis Corp.), Botulinum toxin, Bufferin, Carbatrol®, Carbidopa/levodopa immediate-release (Sinemet), Carbidopa/levodopa oral disintegrating (Parcopa), Carbidopa/levodopa/Entacapone (Stalevo), CERE-110: Adeno-Associated Virus Delivery of NGF (Ceregene), cerebrolysin, CinnoVex, citalopram, citicoline, Clobazam, Clonazepam, Clopidogrel, clozapine (Clozaril), Coenzyme Q, Creatine, dabigatran, dalteparin, Dapsone, Davunetide, Deferiprone, Depakene®, Depakote ER®, Depakote®, Desmoteplase, Diastat, Diazepam, Digoxin, Dilantin®, Dimebon, dipyridamole, divalproex (Depakote), Donepezil (Aricept), EGb 761, Eldepryl, ELND002 (Elan Pharmaceuticals), Enalapril, enoxaparin, Entacapone (Comtan), epoetin alfa, Eptifibatide, Erythropoietin, Escitalopram, Eslicarbazepine acetate, Esmolol, Ethosuximide, Ethyl-EPA (Miraxion™), Exenatide, Extavia, Ezogabine, Felbamate, Felbatol®, Fingolimod (Gilenya), fluoxetine (Prozac), fondaparinux, Fragmin, Frisium, Gabapentin, Gabitril®, Galantamine, Glatiramer (Copaxone), haloperidol (Haldol), Heparin, human chorionic gonadotropin (hCG), Idebenone, Inovelon®, insulin, Interferon beta 1a, Interferon beta 1b, ioflupane 1231 (DATSCAN®), IPX066 (Impax Laboratories Inc.), JNJ-26489112 (Johnson and Johnson), Keppra®, Klonopin, Lacosamide, L-Alpha glycerylphosphorylcholine, Lamictal®, Lamotrigine, Levetiracetam, liraglutide, Lisinopril, Lithium carbonate, Lopressor, Lorazepam, losartan, Lovenox, Lu AA24493, Luminal, LY450139 (Eli Lilly), Lyrica, Masitinib, Mecobalamin, Memantine, methylprednisolone, metoprolol tartrate, Minitran, Minocycline, mirtazapine, Mitoxantrone (Novantrone), Mysoline®, Natalizumab (Tysabri), Neurontin®, Niacinamide, Nitro-Bid, Nitro-Dur, nitroglycerin, Nitrolingual, Nitromist, Nitrostat, Nitro-Time, Norepinephrine (NOR), Carbamazepine, octreotide, Onfi®, Oxcarbazepine, Oxybutinin chloride, PF-04360365 (Pfizer), Phenobarbital, Phenytek®, Phenytoin, piclozotan, Pioglitazone, Plavix, Potiga, Pramipexole (Mirapex), pramlintide, Prednisone, Primidone, Prinivil, probenecid, Propranolol, PRX-00023 (EPIX Pharmaceuticals Inc.), PXT3003, Quinacrine, Ramelteon, Rasagiline (Azilect), Rebif, ReciGen, remacemide, Resveratrol, Retavase, reteplase, riluzole (Rilutek), Rivastigmine (Exelon), Ropinirole (Requip), Rotigotine (Neupro), Rufinamide, Sabril, safinamide (EMD Serono), Salagen, Sarafem, Selegiline (1-deprenyl, Eldepryl), SEN0014196 (Siena Biotech), sertraline (Zoloft), Simvastatin, Sodium Nitroprussiate (NPS), sodium phenylbutyrate, Stanback Headache Powder, Tacrine (Cognex), Tamoxifen, tauroursodeoxycholic acid (TUDCA), Tegretol®, Tenecteplase, Tenormin, Tetrabenazine (Xenazine), THR-18 (Thrombotech Ltd.), Tiagabine, Tideglusib, tirofiban, tissue plasminogen activator (tPA), tizanidine (Zanaflex), TNKase, Tolcapone (Tasmar), Tolterodine, Topamax®, Topiramate, Trihexyphenidyl (formerly Artane), Trileptal®, ursodiol, Valproic Acid, valsartan, Varenicline (Pfizer), Vimpat, Vitamin E, Warfarin, Zarontin®, Zestril, Zonegran®, Zonisamide, Zydis selegiline HCL Oral disintegrating (Zelapar), and combinations thereof.

Another embodiment of the present invention is a method of treating or ameliorating the effects of a condition associated with increased protein disulfide isomerase (PDI) activity in a subject in need thereof comprising administering to the subject an effective amount of a compound selected from the group consisting of formula (I)

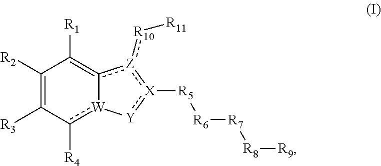

formula (II)

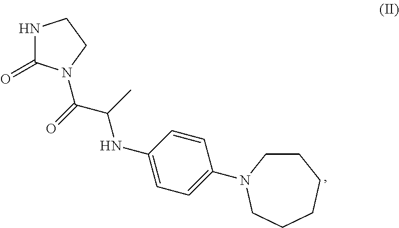 [SMILES: CC(Nc1ccc(N2CCCCCC2)cc1)C(=O)N1CCNC1=O]

formula (III)

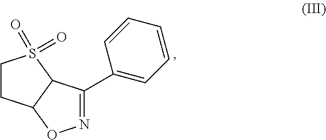 [SMILES: O=S1(=O)CCC2ON=C(c3ccccc3)C21]

and formula (IV):

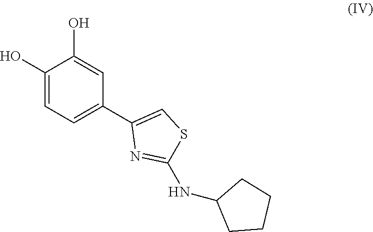 [SMILES: Oc1ccc(-c2csc(NC3CCCC3)n2)cc1O]

wherein a dashed line indicates the presence of an optional double bond,
wherein W, X, Y and Z are independently selected from the group consisting of C, N, S and O,
wherein R₁, R₂, R₃, and R₄ are independently selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₅, R₆, R₇, R₈ and R₁₀ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₉ and R₁₁ are independently selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
or an N-oxide, crystalline form, hydrate thereof, or a pharmaceutically acceptable salt thereof.

In another aspect of this embodiment the subject is a mammal. In some aspects of this embodiment the mammal is selected from the group consisting of humans, veterinary animals, and agricultural animals. In another aspect of this embodiment the subject is a human.

In one aspect of this embodiment the condition is a protein folding disorder. In another aspect of this embodiment the condition is cancer. In yet another aspect of this embodiment the condition is HIV. In yet another aspect of this embodiment the condition is a blood clot.

Another embodiment of the present invention is a method of modulating PDI activity in a cell comprising contacting the cell with an effective amount of a compound selected from the group consisting of formula (I)

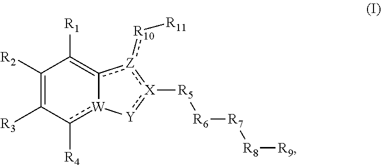

formula (II)

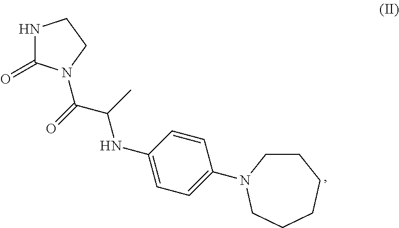 [SMILES: CC(Nc1ccc(N2CCCCCC2)cc1)C(=O)N1CCNC1=O]

formula (III)

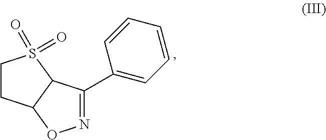 [SMILES: O=S1(=O)CCC2ON=C(c3ccccc3)C21]

and formula (IV):

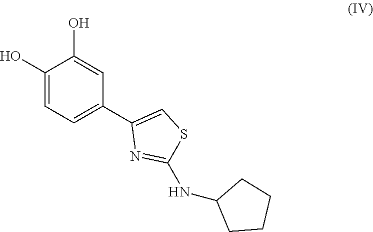 [SMILES: Oc1ccc(-c2csc(NC3CCCC3)n2)cc1O]

wherein a dashed line indicates the presence of an optional double bond,
wherein W, X, Y and Z are independently selected from the group consisting of C, N, S and O,
wherein R₁, R₂, R₃, and R₄ are independently selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₅, R₆, R₇, R₈ and R₁₀ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₉ and R₁₁ are independently selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
or an N-oxide, crystalline form, hydrate thereof, or a pharmaceutically acceptable salt thereof.

Another embodiment of the present invention is a compound having the

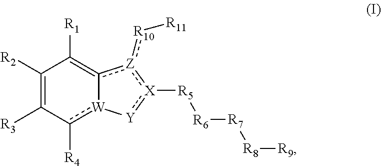

wherein a dashed line indicates the presence of an optional double bond,
wherein W, X, Y and Z are independently selected from the group consisting of C, N, S and O,
wherein R₁, R₂, R₃, and R₄ are independently selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₅, R₆, R₇, R₈ and R₁₀ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₉ and R₁₁ are independently selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
with the proviso that the compound is not

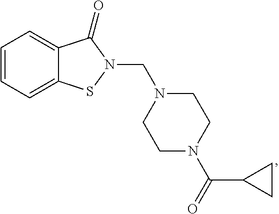 [SMILES: O=C(C1CC1)N1CCN(Cn2sc3ccccc3c2=O)CC1]

or an N-oxide, crystalline form, hydrate thereof, or a pharmaceutically acceptable salt thereof.

Another embodiment of the present invention is a compound having the formula (Ia)

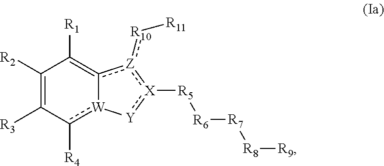

wherein a dashed line indicates the presence of an optional double bond,
wherein W, X, and Z are independently selected from the group consisting of C, N, S and O,
wherein Y is selected from the group consisting of C, N, Se, S and O, or another group VI atom,
wherein R₁, R₂, R₃, and R₄ are independently selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₅, R₆, R₇, R₈ and R₁₀ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₉ and R₁₁ are independently selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
with the proviso that the compound is not

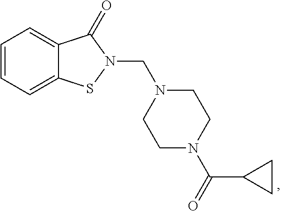 [SMILES: O=C(C1CC1)N1CCN(Cn2sc3ccccc3c2=O)CC1]

or an N-oxide, crystalline form, hydrate thereof, or a pharmaceutically acceptable salt thereof.

Another embodiment of the present invention is a compound having the formula (II)

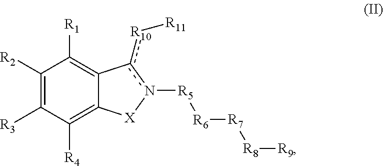

wherein a dashed line indicates the presence of an optional double bond,
wherein X is selected from the group consisting of S and Se,
wherein R₁, R₂, R₃, and R₄ are independently selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₅ is selected from the group consisting of C₁₋₄alkyl, C(O)NH, C(O), C(O)O, NH and O,
wherein R₆, is -(optionally substituted mono- or polycyclic group containing 3 to 20 carbon atoms and optionally 1 to 4 heteroatoms selected from O, N and S),
wherein R₇ and R₈ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₉ is selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₁₀ is selected from the group consisting of no atom and O,
wherein R₁₁ is selected from the group consisting of O and -(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof, with the proviso that the compound is not

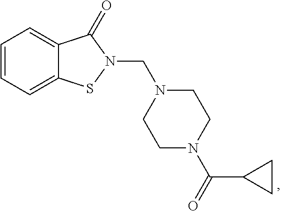 [SMILES: O=C(C1CC1)N1CCN(Cn2sc3ccccc3c2=O)CC1]

or an N-oxide, crystalline form, hydrate thereof, or a pharmaceutically acceptable salt thereof.

In one aspect of this embodiment the compound is

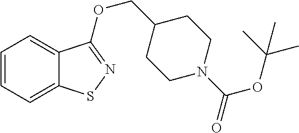 [SMILES: CC(C)(C)OC(=O)N1CCC(COc2nsc3ccccc23)CC1]

or an N-oxide, crystalline form, hydrate thereof, or a pharmaceutically acceptable salt thereof.

Another embodiment of the present invention is a compound having the formula (III)

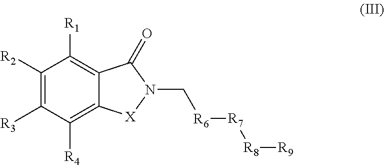

wherein X is selected from the group consisting of S and Se,
wherein R₆, is selected from the group consisting of the group consisting of phenyl,

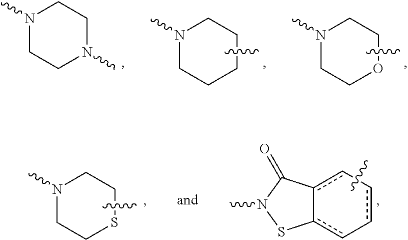

wherein a wavy line indicates an attachment point to the molecule,
wherein R₇ and R₈ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₉ is selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof, with the proviso that the compound is not

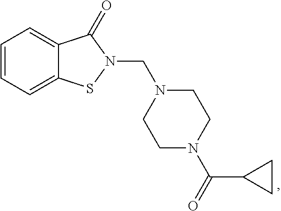 [SMILES: O=C(C1CC1)N1CCN(Cn2sc3ccccc3c2=O)CC1]

or an N-oxide, crystalline form, hydrate thereof, or a pharmaceutically acceptable salt thereof.

In one aspect of this embodiment the compound is selected from the group consisting of

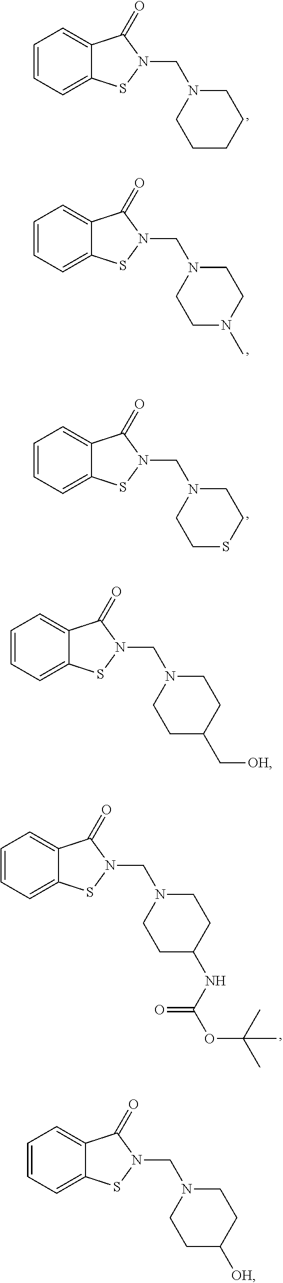 [SMILES: O=c1c2ccccc2sn1CN1CCCCC1 ; CN1CCN(Cn2sc3ccccc3c2=O)CC1 ; O=c1c2ccccc2sn1CN1CCSCC1 ; O=c1c2ccccc2sn1CN1CCC(CO)CC1 ; CC(C)(C)OC(=O)NC1CCN(Cn2sc3ccccc3c2=O)CC1 ; O=c1c2ccccc2sn1CN1CCC(O)CC1]

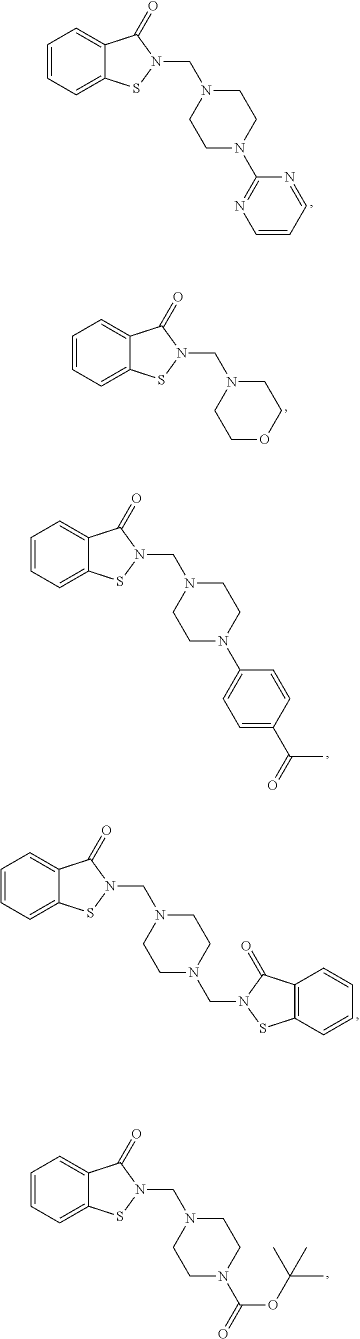 [SMILES: O=c1c2ccccc2sn1CN1CCN(c2ncccn2)CC1 ; O=c1c2ccccc2sn1CN1CCOCC1 ; CC(=O)c1ccc(N2CCN(Cn3sc4ccccc4c3=O)CC2)cc1 ; O=c1c2ccccc2sn1CN1CCN(Cn2sc3ccccc3c2=O)CC1 ; CC(C)(C)OC(=O)N1CCN(Cn2sc3ccccc3c2=O)CC1]

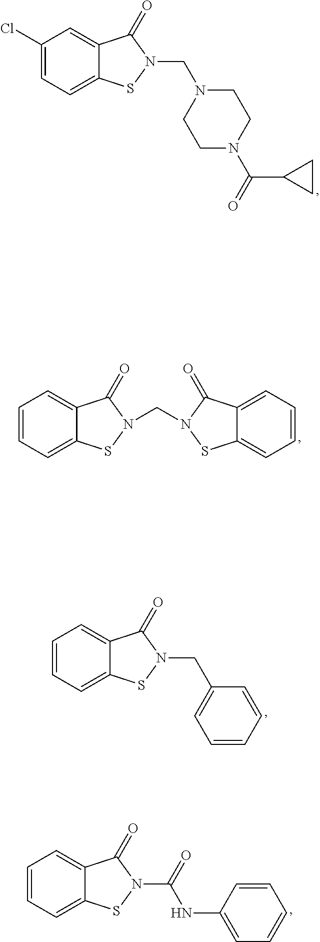 [SMILES: O=C(C1CC1)N1CCN(Cn2sc3ccc(Cl)cc3c2=O)CC1 ; O=c1c2ccccc2sn1Cn1sc2ccccc2c1=O ; O=c1c2ccccc2sn1Cc1ccccc1 ; O=C(Nc1ccccc1)n1sc2ccccc2c1=O]

and combinations thereof.

Another embodiment of the present invention is a composition comprising a compound of the present invention and a pharmaceutically acceptable carrier, adjuvant or vehicle.

Another embodiment of the present invention is a pharmaceutically acceptable salt of a compound of the present invention.

Another embodiment of the present invention is a composition comprising a pharmaceutically acceptable salt of the present invention and a pharmaceutically acceptable carrier, adjuvant or vehicle.

Another embodiment of the invention is a kit comprising a compound or composition of the present invention and instructions for use.

In one aspect of this embodiment, the instructions for use are instructions for treating or ameliorating the effects of a neurodegenerative disorder in a subject. In another aspect of this embodiment, the instruction for use are instructions for treating or ameliorating the effects of a condition associated with increased protein disulfide isomerase (PDI) activity in a subject. In another aspect of this embodiment, the instructions for use are instructions for modulating PDI activity in a cell.

Another embodiment of the invention is a method for treating or ameliorating the effects of a neurodegenerative disorder in a subject in need thereof comprising administering to the subject an effective amount of a compound selected from the group consisting of formula (Ia)

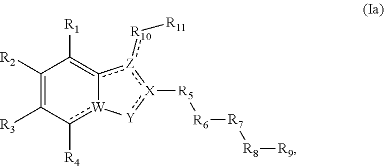

wherein a dashed line indicates the presence of an optional double bond,
wherein W, X, and Z are independently selected from the group consisting of C, N, S and O,
wherein Y is selected from the group consisting of C, N, Se, S and O, or another group VI atom,
wherein R₁, R₂, R₃, and R₄ are independently selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₅, R₆, R₇, R₈ and R₁₀ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₉ and R₁₁ are independently selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
or an N-oxide, crystalline form, hydrate thereof, or a pharmaceutically acceptable salt thereof;
formula (II)

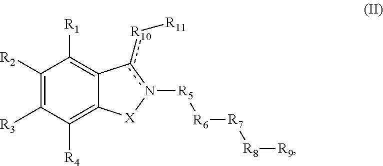

wherein a dashed line indicates the presence of an optional double bond,
wherein X is selected from the group consisting of S and Se,
wherein R₁, R₂, R₃, and R₄ are independently selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₅ is selected from the group consisting of C₁₋₄alkyl, C(O)NH, C(O), C(O)O, NH and O,
wherein R₆, is -(optionally substituted mono- or polycyclic group containing 3 to 20 carbon atoms and optionally 1 to 4 heteroatoms selected from O, N and S),
wherein R₇ and R₈ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₉ is selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₁₀ is selected from the group consisting of no atom and O,
wherein R₁₁ is selected from the group consisting of O and -(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
or an N-oxide, crystalline form, hydrate thereof, or a pharmaceutically acceptable salt thereof; and

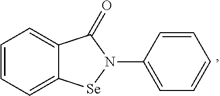 [SMILES: O=c1c2ccccc2[se]n1-c1ccccc1]

or an N-oxide, crystalline form, hydrate thereof, or a pharmaceutically acceptable salt thereof.

Another embodiment of the invention is a method for treating or ameliorating the effects of a neurodegenerative disorder in a subject in need thereof comprising administering to the subject an effective amount of a composition of the invention.

In one aspect of the above embodiments the neurodegenerative disease is selected from the group consisting of Alzheimer's disease, Parkinson's disease, amyotrophic lateral sclerosis (ALS), Friedreich's ataxia, multiple sclerosis, Huntington's Disease, transmissible spongiform encephalopathy, Charcot-Marie-Tooth disease, dementia with Lewy bodies, corticobasal degeneration, progressive supranuclear palsy, and hereditary spastic paraparesis. In a preferred embodiment, the neurodegenerative disease is Huntington's Disease.

In one aspect of the above embodiments the subject is a mammal. In some aspects of this embodiment the mammal is selected from the group consisting of humans, veterinary animals, and agricultural animals. In another aspect of this embodiment the subject is a human.

In one aspect of the above embodiments the method further comprises co-administering to the subject an effective amount of one or more additional therapeutic agents. Preferably, the one or more additional therapeutic agents are selected from the group consisting of 5-hydroxytryptophan, Activase, AFQ056 (Novartis), Aggrastat, Albendazole, alpha-lipoic acid/L-acetyl carnitine, Alteplase, Amantadine (Symmetrel), amlodipine, Ancrod, Apomorphine (Apokyn), Arimoclomol, Arixtra, Armodafinil, Ascorbic acid, Ascriptin, Aspirin, atenolol, Avonex, baclofen (Lioresal), Banzel, Benztropine (Cogentin), Betaseron, BGG492 (Novartis Corp.), Botulinum toxin, Bufferin, Carbatrol®, Carbidopa/levodopa immediate-release (Sinemet), Carbidopa/levodopa oral disintegrating (Parcopa), Carbidopa/levodopa/Entacapone (Stalevo), CERE-110: Adeno-Associated Virus Delivery of NGF (Ceregene), cerebrolysin, CinnoVex, citalopram, citicoline, Clobazam, Clonazepam, Clopidogrel, clozapine (Clozaril), Coenzyme Q, Creatine, dabigatran, dalteparin, Dapsone, Davunetide, Deferiprone, Depakene®, Depakote ER®, Depakote®, Desmoteplase, Diastat, Diazepam, Digoxin, Dilantin®, Dimebon, dipyridamole, divalproex (Depakote), Donepezil (Aricept), EGb 761, Eldepryl, ELND002 (Elan Pharmaceuticals), Enalapril, enoxaparin, Entacapone (Comtan), epoetin alfa, Eptifibatide, Erythropoietin, Escitalopram, Eslicarbazepine acetate, Esmolol, Ethosuximide, Ethyl-EPA (Miraxion™), Exenatide, Extavia, Ezogabine, Felbamate, Felbatol®, Fingolimod (Gilenya), fluoxetine (Prozac), fondaparinux, Fragmin, Frisium, Gabapentin, Gabitril®, Galantamine, Glatiramer (Copaxone), haloperidol (Haldol), Heparin, human chorionic gonadotropin (hCG), Idebenone, Inovelon®, insulin, Interferon beta 1a, Interferon beta 1b, ioflupane 1231 (DATSCAN®), IPX066 (Impax Laboratories Inc.), JNJ-26489112 (Johnson and Johnson), Keppra®, Klonopin, Lacosamide, L-Alpha glycerylphosphorylcholine, Lamictal®, Lamotrigine, Levetiracetam, liraglutide, Lisinopril, Lithium carbonate, Lopressor, Lorazepam, losartan, Lovenox, Lu AA24493, Luminal, LY450139 (Eli Lilly), Lyrica, Masitinib, Mecobalamin, Memantine, methylprednisolone, metoprolol tartrate, Minitran, Minocycline, mirtazapine, Mitoxantrone (Novantrone), Mysoline®, Natalizumab (Tysabri), Neurontin®, Niacinamide, Nitro-Bid, Nitro-Dur, nitroglycerin, Nitrolingual, Nitromist, Nitrostat, Nitro-Time, Norepinephrine (NOR), Carbamazepine, octreotide, Onfi®, Oxcarbazepine, Oxybutinin chloride, PF-04360365 (Pfizer), Phenobarbital, Phenytek®, Phenytoin, piclozotan, Pioglitazone, Plavix, Potiga, Pramipexole (Mirapex), pramlintide, Prednisone, Primidone, Prinivil, probenecid, Propranolol, PRX-00023 (EPIX Pharmaceuticals Inc.), PXT3003, Quinacrine, Ramelteon, Rasagiline (Azilect), Rebif, ReciGen, remacemide, Resveratrol, Retavase, reteplase, riluzole (Rilutek), Rivastigmine (Exelon), Ropinirole (Requip), Rotigotine (Neupro), Rufinamide, Sabril, safinamide (EMD Serono), Salagen, Sarafem, Selegiline (1-deprenyl, Eldepryl), SEN0014196 (Siena Biotech), sertraline (Zoloft), Simvastatin, Sodium Nitroprussiate (NPS), sodium phenylbutyrate, Stanback Headache Powder, Tacrine (Cognex), Tamoxifen, tauroursodeoxycholic acid (TUDCA), Tegretol®, Tenecteplase, Tenormin, Tetrabenazine (Xenazine), THR-18 (Thrombotech Ltd.), Tiagabine, Tideglusib, tirofiban, tissue plasminogen activator (tPA), tizanidine (Zanaflex), TNKase, Tolcapone (Tasmar), Tolterodine, Topamax®, Topiramate, Trihexyphenidyl (formerly Artane), Trileptal®, ursodiol, Valproic Acid, valsartan, Varenicline (Pfizer), Vimpat, Vitamin E, Warfarin, Zarontin®, Zestril, Zonegran®, Zonisamide, Zydis selegiline HCL Oral disintegrating (Zelapar), and combinations thereof.

In one aspect of the above embodiments the compound is selected from the group consisting of

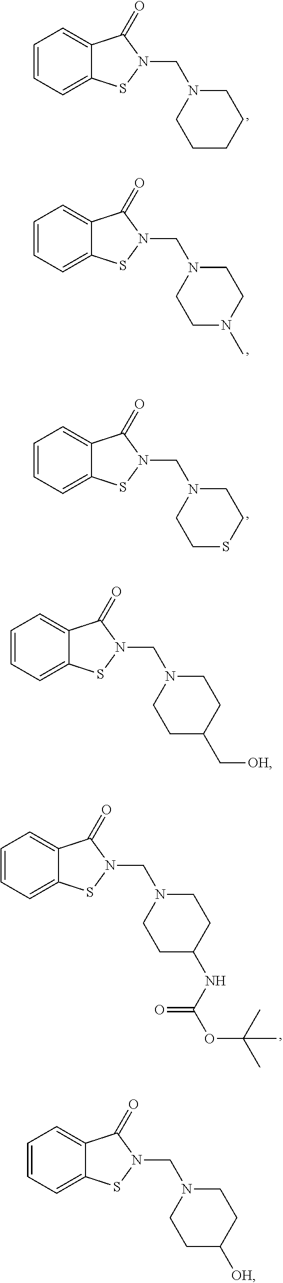 [SMILES: O=c1c2ccccc2sn1CN1CCCCC1 ; CN1CCN(Cn2sc3ccccc3c2=O)CC1 ; O=c1c2ccccc2sn1CN1CCSCC1 ; O=c1c2ccccc2sn1CN1CCC(CO)CC1 ; CC(C)(C)OC(=O)NC1CCN(Cn2sc3ccccc3c2=O)CC1 ; O=c1c2ccccc2sn1CN1CCC(O)CC1]

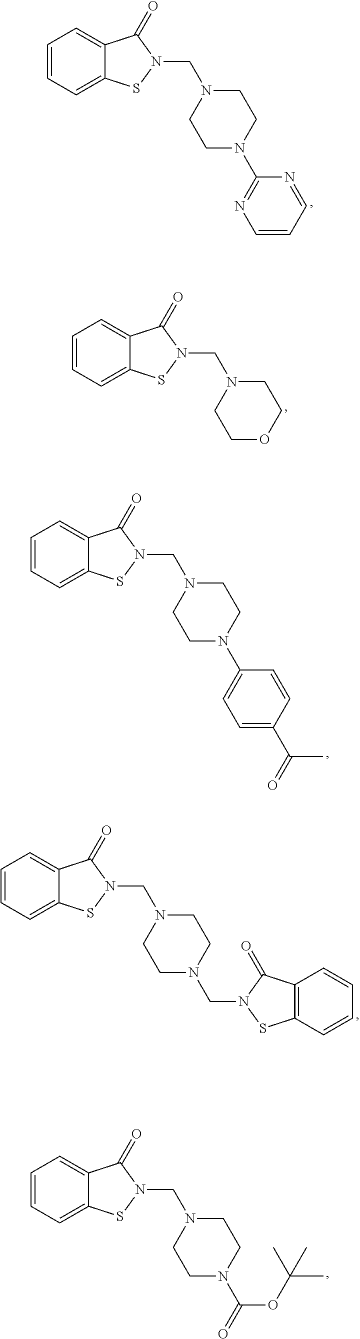 [SMILES: O=c1c2ccccc2sn1CN1CCN(c2ncccn2)CC1 ; O=c1c2ccccc2sn1CN1CCOCC1 ; CC(=O)c1ccc(N2CCN(Cn3sc4ccccc4c3=O)CC2)cc1 ; O=c1c2ccccc2sn1CN1CCN(Cn2sc3ccccc3c2=O)CC1 ; CC(C)(C)OC(=O)N1CCN(Cn2sc3ccccc3c2=O)CC1]

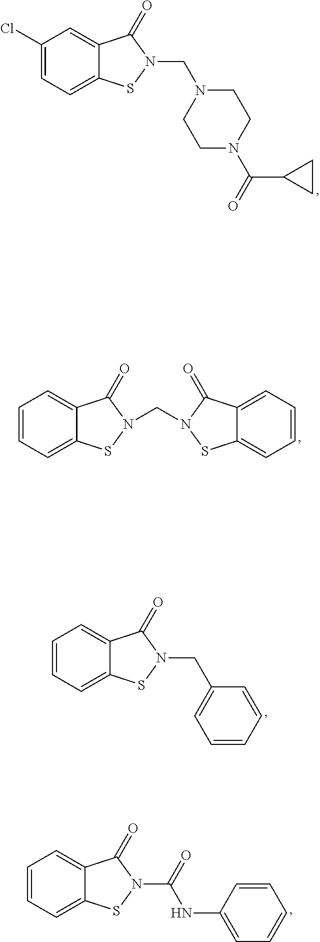 [SMILES: O=C(C1CC1)N1CCN(Cn2sc3ccc(Cl)cc3c2=O)CC1 ; O=c1c2ccccc2sn1Cn1sc2ccccc2c1=O ; O=c1c2ccccc2sn1Cc1ccccc1 ; O=C(Nc1ccccc1)n1sc2ccccc2c1=O]

and combinations thereof.

Another embodiment of the invention is a method for treating or ameliorating the effects of a condition associated with increased protein disulfide isomerase (PDI) activity in a subject in need thereof comprising administering to the subject an effective amount of a compound selected from the group consisting of formula (Ia)

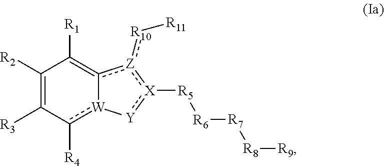

wherein a dashed line indicates the presence of an optional double bond,
wherein W, X, and Z are independently selected from the group consisting of C, N, S and O,
wherein Y is selected from the group consisting of C, N, Se, S and O, or another group VI atom,
wherein R₁, R₂, R₃, and R₄ are independently selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₅, R₆, R₇, R₈ and R₁₀ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₉ and R₁₁ are independently selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof, or an N-oxide, crystalline form, hydrate thereof, or a pharmaceutically acceptable salt thereof;

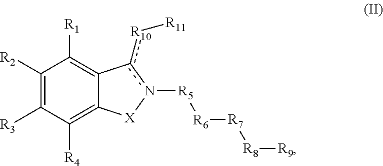

wherein a dashed line indicates the presence of an optional double bond,
wherein X is selected from the group consisting of S and Se,
wherein R₁, R₂, R₃, and R₄ are independently selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₅ is selected from the group consisting of C₁₋₄alkyl, C(O)NH, C(O), C(O)O, NH and O,
wherein R₆, is -(optionally substituted mono- or polycyclic group containing 3 to 20 carbon atoms and optionally 1 to 4 heteroatoms selected from O, N and S),
wherein R₇ and R₈ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₉ is selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₁₀ is selected from the group consisting of no atom and O,
wherein R₁₁ is selected from the group consisting of O and -(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
or an N-oxide, crystalline form, hydrate thereof, or a pharmaceutically acceptable salt thereof; and

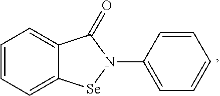 [SMILES: O=c1c2ccccc2[se]n1-c1ccccc1]

or an N-oxide, crystalline form, hydrate thereof, or a pharmaceutically acceptable salt thereof.

Another embodiment of the invention is a method for treating or ameliorating the effects of a condition associated with increased protein disulfide isomerase (PDI) activity in a subject in need thereof comprising administering to the subject an effective amount of a composition of the present invention.

In one aspect of the above embodiments the subject is a mammal. In some aspects of this embodiment the mammal is selected from the group consisting of humans, veterinary animals, and agricultural animals. In another aspect of this embodiment the subject is a human.

In one aspect of the above embodiments the condition is selected from the group consisting of a protein folding disorder, cancer, HIV, and a blood clot.

In one aspect of the above embodiments the compound is selected from the group consisting of

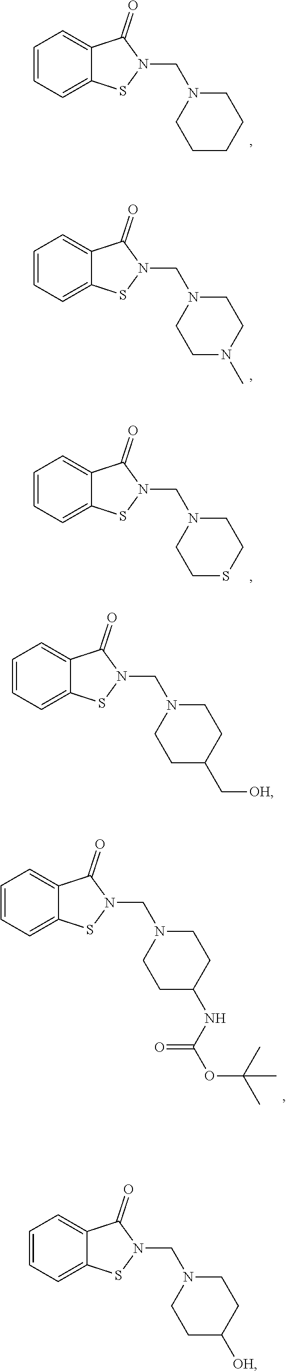 [SMILES: O=c1c2ccccc2sn1CN1CCCCC1 ; CN1CCN(Cn2sc3ccccc3c2=O)CC1 ; O=c1c2ccccc2sn1CN1CCSCC1 ; O=c1c2ccccc2sn1CN1CCC(CO)CC1 ; CC(C)(C)OC(=O)NC1CCN(Cn2sc3ccccc3c2=O)CC1 ; O=c1c2ccccc2sn1CN1CCC(O)CC1]

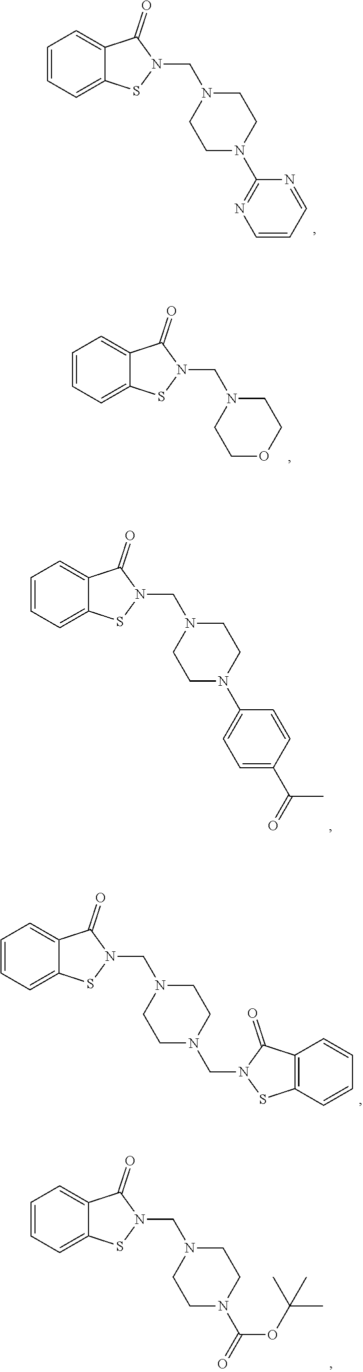 [SMILES: O=c1c2ccccc2sn1CN1CCN(c2ncccn2)CC1 ; O=c1c2ccccc2sn1CN1CCOCC1 ; CC(=O)c1ccc(N2CCN(Cn3sc4ccccc4c3=O)CC2)cc1 ; O=c1c2ccccc2sn1CN1CCN(Cn2sc3ccccc3c2=O)CC1 ; CC(C)(C)OC(=O)N1CCN(Cn2sc3ccccc3c2=O)CC1]

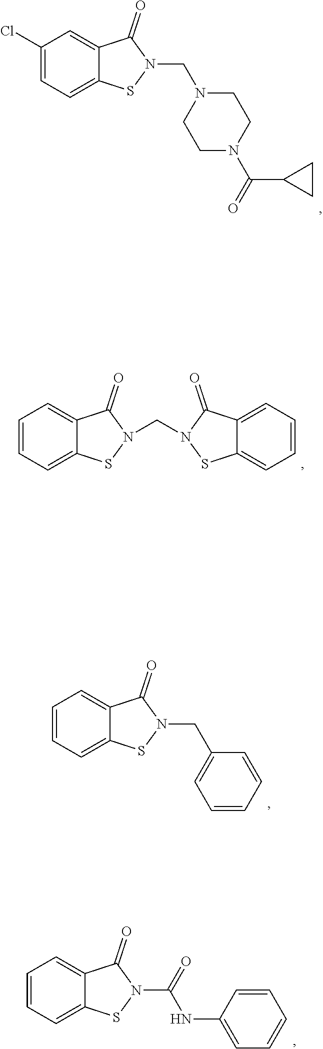 [SMILES: O=C(C1CC1)N1CCN(Cn2sc3ccc(Cl)cc3c2=O)CC1 ; O=c1c2ccccc2sn1Cn1sc2ccccc2c1=O ; O=c1c2ccccc2sn1Cc1ccccc1 ; O=C(Nc1ccccc1)n1sc2ccccc2c1=O]

and combinations thereof.

Another embodiment of the invention is a method of modulating PDI activity in a cell comprising administering to the subject an effective amount of a compound selected from the group consisting of formula (Ia)

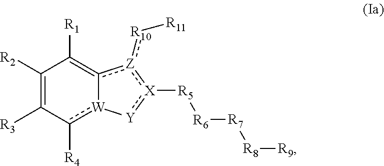

wherein a dashed line indicates the presence of an optional double bond,
wherein W, X, and Z are independently selected from the group consisting of C, N, S and O,
wherein Y is selected from the group consisting of C, N, Se, S and O, or another group VI atom,
wherein R₁, R₂, R₃, and R₄ are independently selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₅, R₆, R₇, R₈ and R₁₀ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₉ and R₁₁ are independently selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
or an N-oxide, crystalline form, hydrate thereof, or a pharmaceutically acceptable salt thereof;
formula (II)

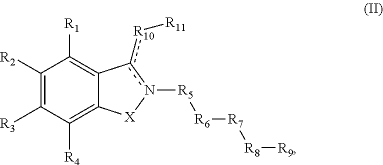

wherein a dashed line indicates the presence of an optional double bond,
wherein X is selected from the group consisting of S and Se,
wherein R₁, R₂, R₃, and R₄ are independently selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₅ is selected from the group consisting of C₁₋₄alkyl, C(O)NH, C(O), C(O)O, NH and O,
wherein R₆, is -(optionally substituted mono- or polycyclic group containing 3 to 20 carbon atoms and optionally 1 to 4 heteroatoms selected from O, N and S),
wherein R₇ and R₈ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₉ is selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₁₀ is selected from the group consisting of no atom and O,
wherein R₁₁ is selected from the group consisting of O and -(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
or an N-oxide, crystalline form, hydrate thereof, or a pharmaceutically acceptable salt thereof; and

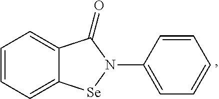 [SMILES: O=c1c2ccccc2[se]n1-c1ccccc1]

or an N-oxide, crystalline form, hydrate thereof, or a pharmaceutically acceptable salt thereof.

Another embodiment of the invention is a method of modulating PDI activity in a cell comprising administering to the subject an effective amount of a composition of the present invention.

In one aspect of the above embodiments the compound is selected from the group consisting of

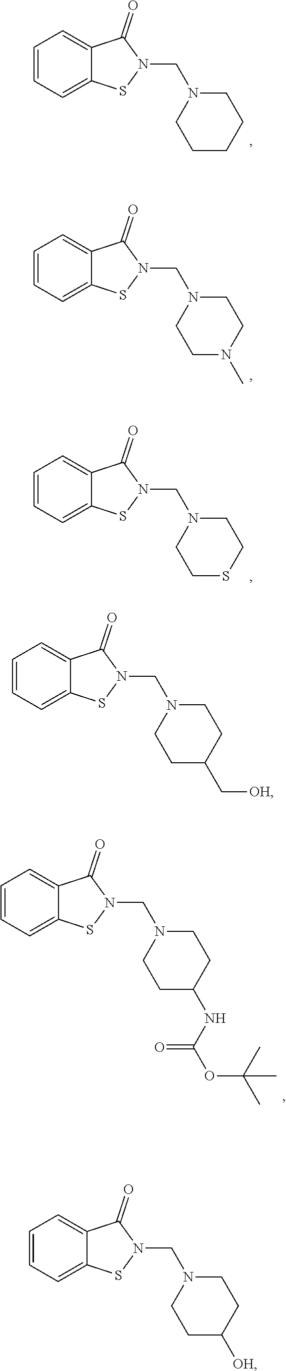 [SMILES: O=c1c2ccccc2sn1CN1CCCCC1 ; CN1CCN(Cn2sc3ccccc3c2=O)CC1 ; O=c1c2ccccc2sn1CN1CCSCC1 ; O=c1c2ccccc2sn1CN1CCC(CO)CC1 ; CC(C)(C)OC(=O)NC1CCN(Cn2sc3ccccc3c2=O)CC1 ; O=c1c2ccccc2sn1CN1CCC(O)CC1]

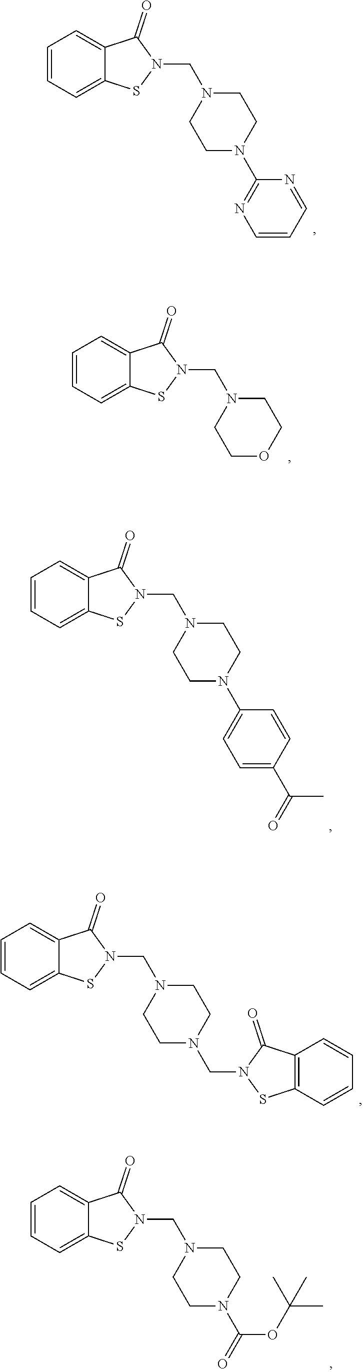 [SMILES: O=c1c2ccccc2sn1CN1CCN(c2ncccn2)CC1 ; O=c1c2ccccc2sn1CN1CCOCC1 ; CC(=O)c1ccc(N2CCN(Cn3sc4ccccc4c3=O)CC2)cc1 ; O=c1c2ccccc2sn1CN1CCN(Cn2sc3ccccc3c2=O)CC1 ; CC(C)(C)OC(=O)N1CCN(Cn2sc3ccccc3c2=O)CC1]

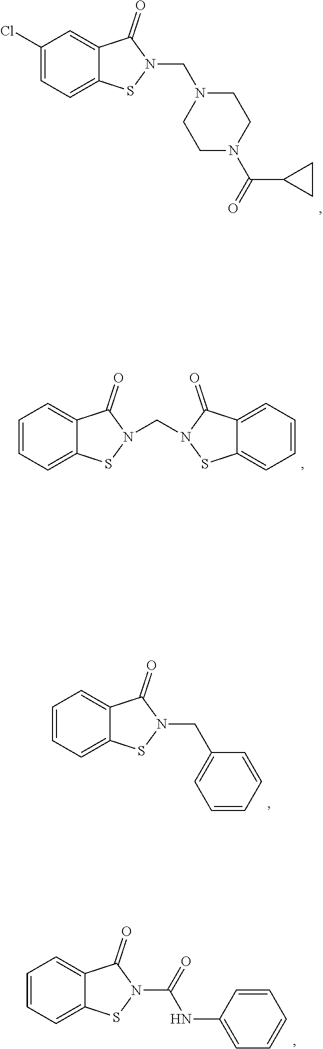 [SMILES: O=C(C1CC1)N1CCN(Cn2sc3ccc(Cl)cc3c2=O)CC1 ; O=c1c2ccccc2sn1Cn1sc2ccccc2c1=O ; O=c1c2ccccc2sn1Cc1ccccc1 ; O=C(Nc1ccccc1)n1sc2ccccc2c1=O]

and combinations thereof.

Another embodiment of the invention is a method for treating or ameliorating the effects of a neurodegenerative disorder in a subject in need thereof comprising administering to the subject an effective amount of a compound selected from the group consisting of:

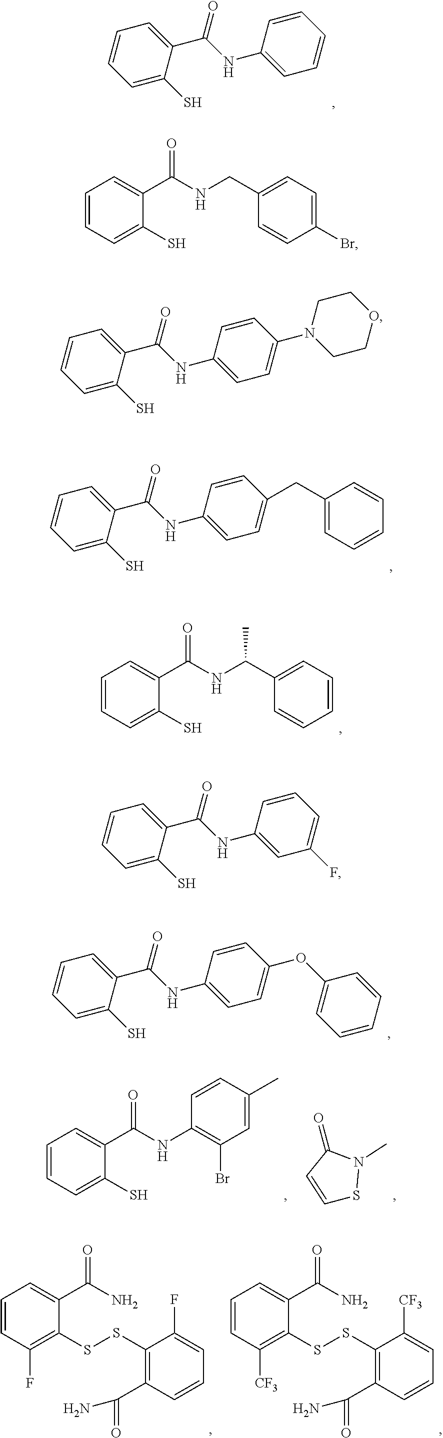

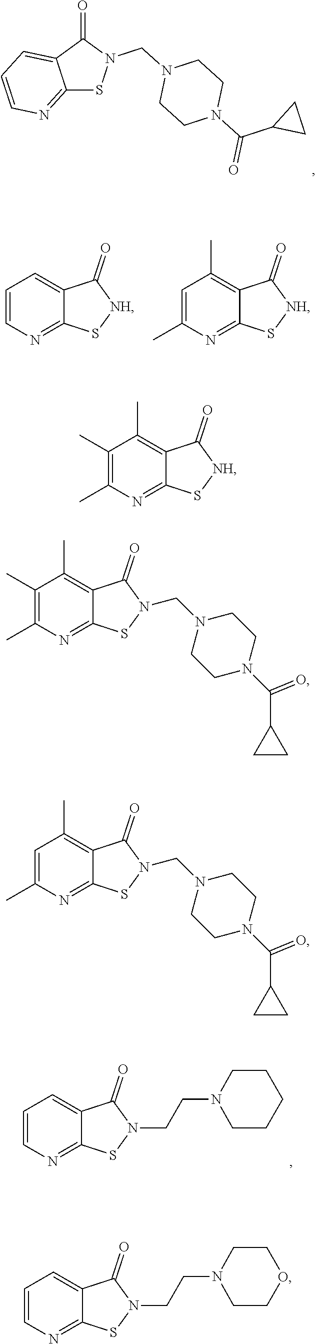

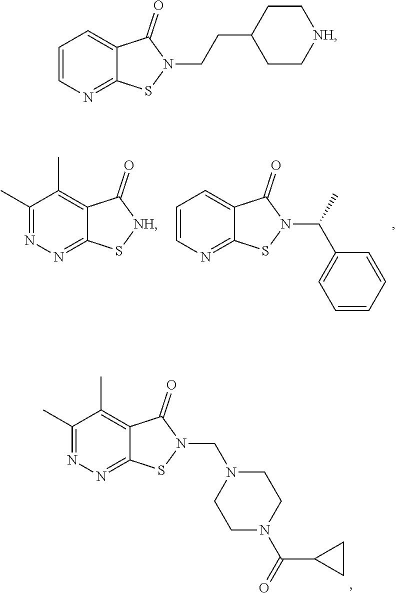

combinations thereof, or an N-oxide, crystalline form, hydrate thereof, or a pharmaceutically acceptable salt thereof.

Another embodiment of the invention is a method for treating or ameliorating the effects of a condition associated with increased protein disulfide isomerase (PDI) activity in a subject in need thereof comprising administering to the subject an effective amount of a compound selected from the group consisting of:

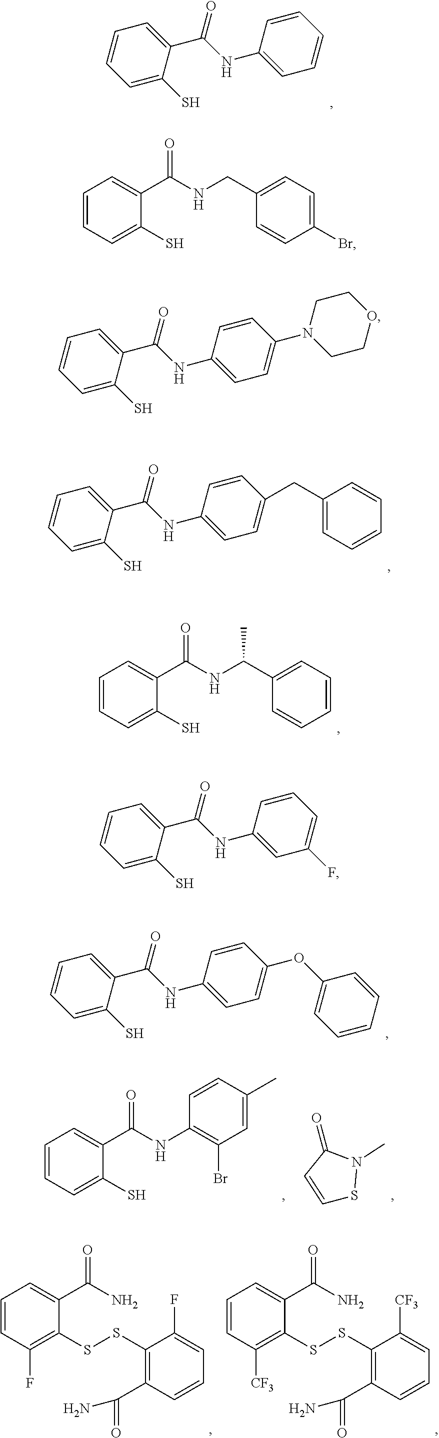

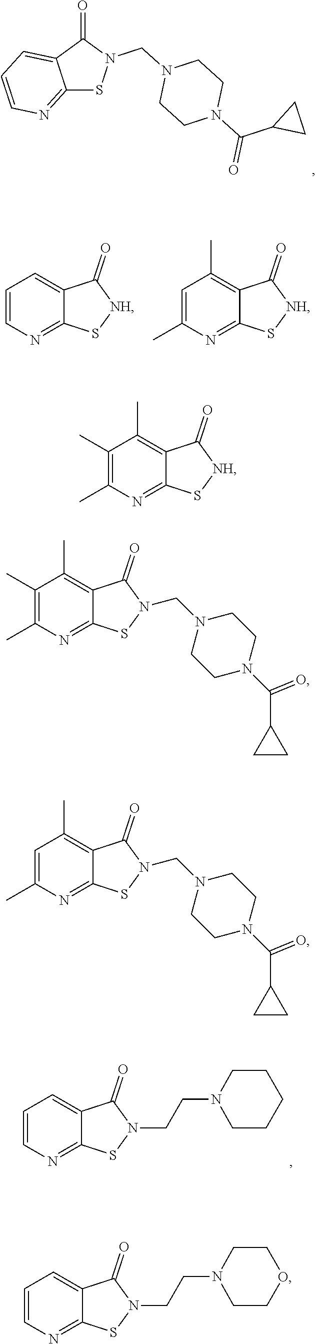

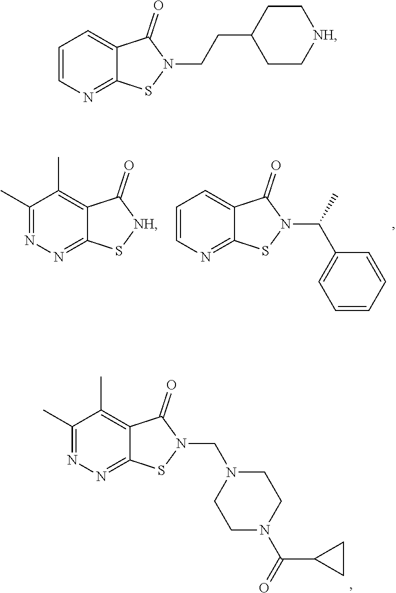

combinations thereof, or an N-oxide, crystalline form, hydrate thereof, or a pharmaceutically acceptable salt thereof.

Another embodiment of the invention is a method of modulating PDI activity in a cell comprising contacting the cell with an effective amount of a compound selected from the group consisting of:

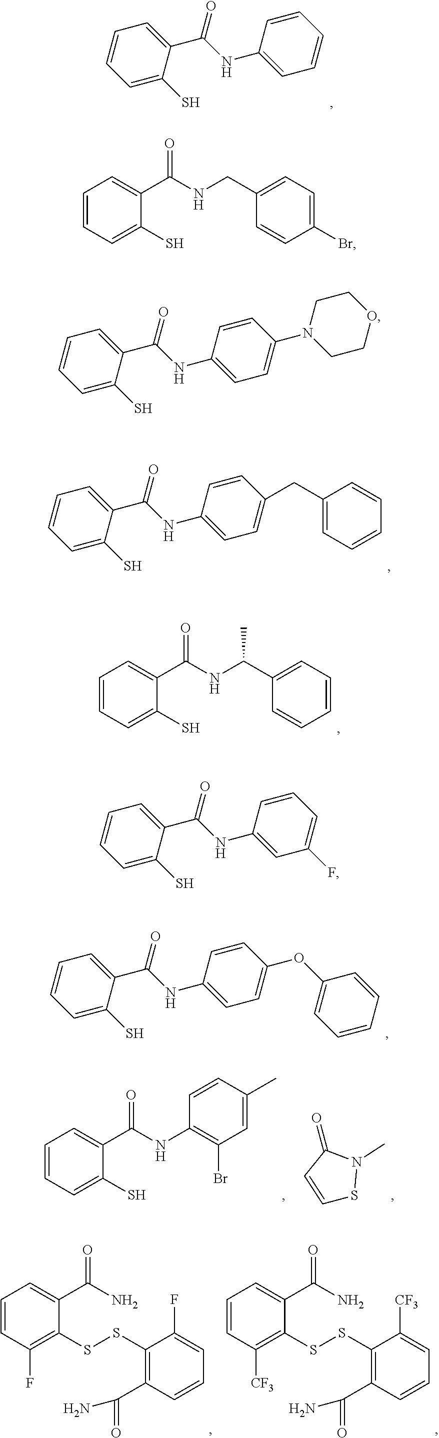

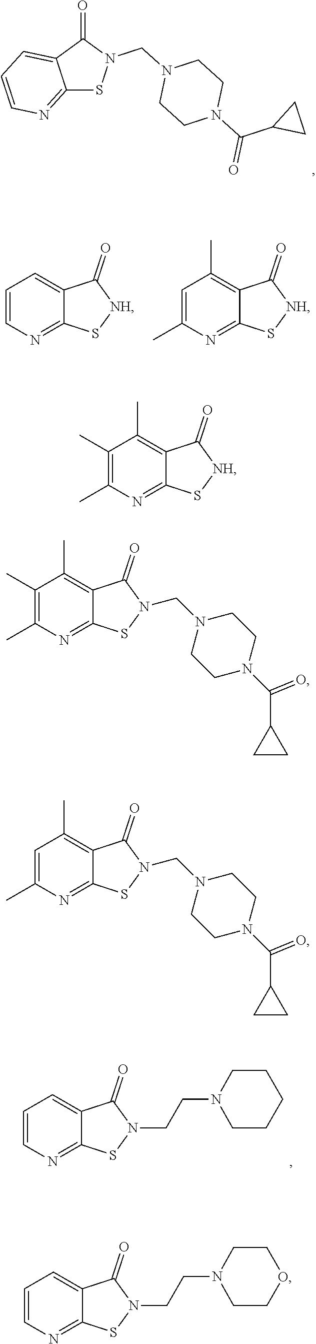

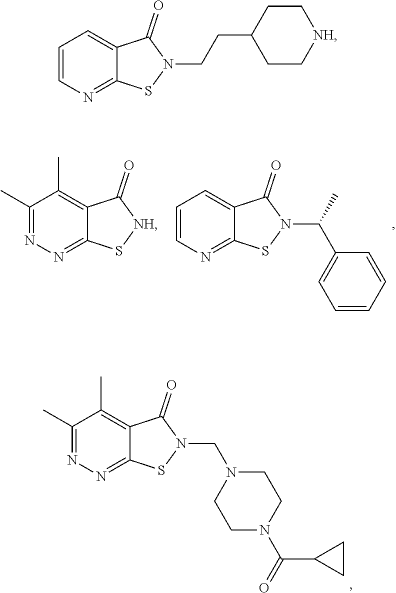

combinations thereof, or an N-oxide, crystalline form, hydrate thereof, or a pharmaceutically acceptable salt thereof.

Another embodiment of the invention is a compound selected from the group consisting of:

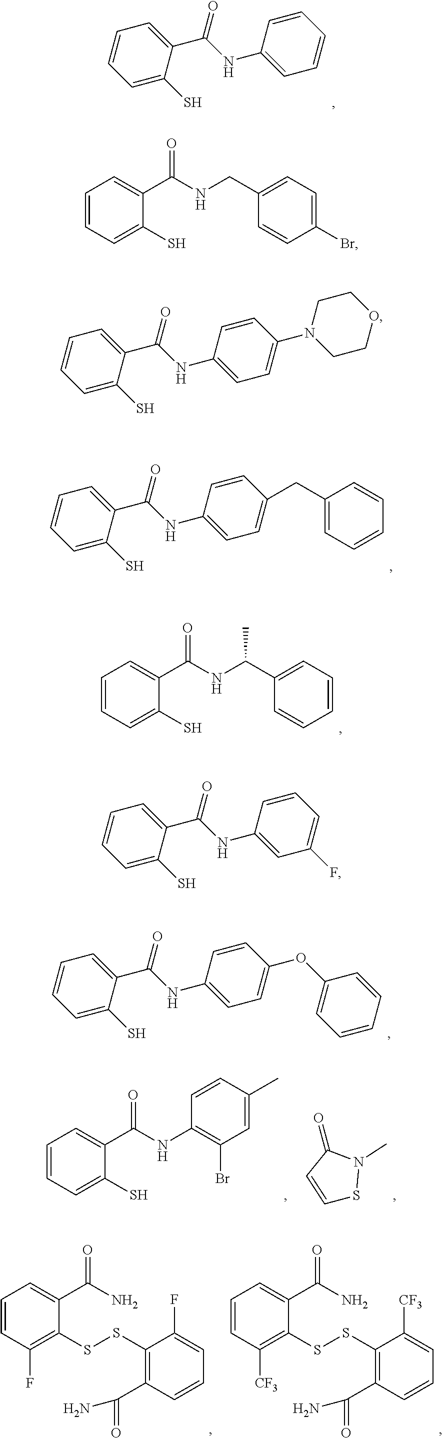

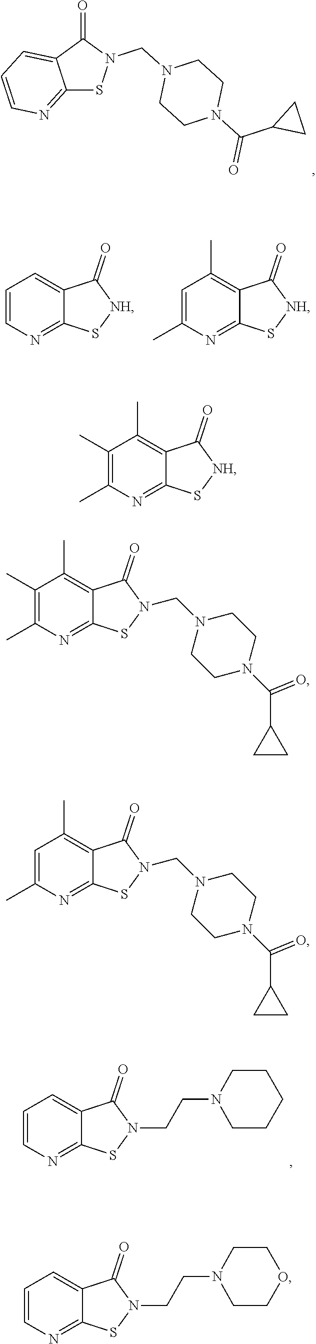

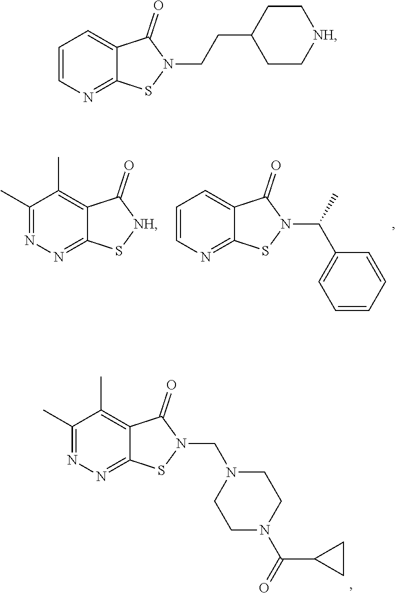

combinations thereof, or an N-oxide, crystalline form, hydrate thereof, or a pharmaceutically acceptable salt thereof. Compositions of the foregoing embodiments are also provided wherein one or more of the foregoing compounds are combined with a pharmaceutically acceptable carrier, adjuvant or vehicle.

In the various embodiments of the present invention, compounds may be recited as a group. It is to be understood that the present invention includes each individual compound alone or in any possible grouping of one, two, three or more compounds.

In the present invention, and as noted in the Examples, the residue numbering is based on the sequence of the mature PDI protein, dapeeedhvlvlrksnfaealaahkyllvefyapwcghckalapeyakaagklkaegseirlakvdateesdlaqqygvr gyptikffrngdtaspkeytagreaddivnwlkkrtgpaattlpdgaaaeslvessevavigffkdvesdsakqflqaaeaid dipfgitsnsdvfskyqldkdgvvlfkkfdegrnnfegevtkenlldfikhnqlplviefteqtapkifggeikthllflpksvsdyd gklsnfktaaesfkgkilfifidsdhtdnqrileffglkkeecpavrlitleeemtkykpeseeltaeritefchrflegkikphlmsq elpedwdkqpvkvlvgknfedvafdekknvfvefyapwcghckqlapiwdklgetykdheniviakmdstaneveavk vhsfptlkffpasadrtvidyngertldgfkkflesggqdgagddddledleeaeepdmeedddqkavkdel, (SEQ ID NO: 3) i.e., residue 1 of the mature PDI corresponds to residue 18 in the full length PDI. The first 17 amino acids in full length PDI are the signal sequence that is processed out to generate the mature PDI.

The following examples are provided to further illustrate certain aspects of the present invention. These examples are illustrative only and are not intended to limit the scope of the invention in any way.

### EXAMPLES

### Example 1

### Materials and Methods

### Cell Culture

PC12 mHTT^{Q103} cells were a gift from Erik S. Schweitzer (UCLA School of Medicine, Los Angeles, Calif.). These cells are stably transfected with the first exon of human HTT gene containing the pathogenic 103 CAG/CAA repeat expansion, under the control of the ecdysteroid promoter (Aiken et al., 2004). The plasmid also contains a Bombyx mori ecdysone receptor gene fused at N-terminal with VP16 transactivation domain (Suhr et al., 1998; Vilaboa et al., 2011). Addition of the ecdysone analog, tebufenozide, to the cell culture medium is used to initiate the transcription of mutant HTT (Aiken et al., 2004).

PC12 mHTT^{Q103} cells were cultured in DMEM containing 4.5 g/I glucose, 25 mM HEPES, sodium pyruvate, and no L-glutamine (Mediatech, cat. no. 15-018-CV), supplemented with 10% (v/v) Cosmic Calf serum, 2 mM L-glutamine, 100 units/mL of penicillin-streptomycin, and 0.5 mg/ml active geneticin. Cells were grown at 37° C., 9.5% CO₂, and the medium was replaced with fresh medium every 2-3 days. To induce mHTT^{Q103} expression for experiments, tebufenozide, a gift from Lynne Moore and Fred H. Gage (The Salk Institute for Biological Studies, La Jolla, Calif.), was added to the medium at 200 nM final concentration from 1 mM stock in 85% ethanol.

### High-Throughput Screen of LOC Library

LOC mother plates with compounds at 4 mg/ml were thawed and spun down (1000 rpm, 20° C., 1 min) prior to use. Biomek FX (Beckman Coulter) robotic liquid dispenser was used to handle all liquid transferring and mixing. Replica daughter plates (D1) were prepared by transferring 2 μl of compound from the mother plate into 384-deep-well clear, round bottom, polypropylene plates (Grenier cat. no. 781270) containing 98 μl of PC12 medium without selective agent geneticin to obtain compound concentrations at 80 μl/ml in 2% DMSO. Two fold serial dilution was performed across five daughter plates by transferring 50 μl of compounds (at 80 μl/ml) from the D1 plate into 50 μl of PC12 medium in daughter plate D2, mixing, and then repeating the process for the remaining three plates. Daughter plate D1 with compounds at 80 μl/ml, daughter plate D3 with compounds at 20 μl/ml and daughter plate D5 with compounds at 5 μl/ml were then used for the screen. Assay plates were set up by seeding tebufenozide-induced PC12 mHTT^{Q103} cells into 384-well black, clear-bottom plates (Corning Inc. cat. no. 3712) at a density of 7,500 cells per well in 57 μl PC12 medium without geneticin. Three microliters of compound from the daughter plates (D1, D3, and D5) were added to the assay plates for a final compound concentration of 4 μl/ml, 1 μl/ml, and 0.25 μl/ml. Four wells containing uninduced PC12 mHTT^{Q103} cells and four wells containing medium only, were also included on each plate as controls. The assay plates were incubated at 37° C., 9.5% CO₂ for 48 hours. Twenty microliters of 40% Alamar blue (Life Technologies cat. no. DAL1100) solution in PC12 medium was added to each well (1:10 final dilution) and the plates were incubated for an additional 12-24 hours at 37° C., 9.5% CO₂. Alamar blue fluorescence was read on a fluorescence plate reader (PerkinElmer Victor3) with 530 nm excitation filter and 590 nm emission filter. Each compound concentration was tested in triplicate.

The follow-up testing of primary screen hits was performed in a similar way. Fresh powder stocks of primary hits were re-ordered from vendors, dissolved in DMSO and tested in a two-fold serial dilution across 10 wells in both tebufenozide-induced and uninduced PC12 mHTT^{Q103} cells. Dose-response curves were plotted as mean±SD and fit to either four-parameter sigmoidal (for uninduced cells) or bell-shaped (induced) function using Prism (GraphPad Software).

### Molecular Cloning, Protein Expression and Purification of PDIa

Sequence for the human catalytic PDI A1 a domain (PDIa) (SEQ ID NO: 3) was generated by PCR from Ultimate ORF Clone IOH9865 (Life Technologies) as an Nde I-BamH I fragment. The amplified a domain (amino acids 18-134 in the full length PDI sequence) was then subcloned into Nde I-BamH I sites of pET-15b vector (Novagen) containing the N-terminal His₆ tag and confirmed by DNA sequencing (GeneWiz, Inc.).

The PDIa construct was transformed into Escherichia coli BL21-Gold (DE3) competent cells (Agilent Technologies) and grown at 37° C. in LB medium with 100 μg/ml ampicillin until OD_{600 nm} reached 0.5. Expression was induced with 0.5 mM IPTG at 37° C. for overnight (usually 12-15 hr). Cells were pelleted (4,000×g, 20 minutes at 4° C.) and lysed by sonication in buffer containing 50 mM Tris-HCl, pH 8.0, 150 mM NaCl, 1 mM TCEP and 5 mM MgCl₂. Cell lysate was then centrifuged at 12,000×rpm for 30 minutes at 4° C. The supernatant was loaded onto a chromatography column containing Ni Sepharose 6 Fast Flow beads (GE Life Sciences) equilibrated with PDI Suspension Buffer (50 mM Tris-HCl, pH 8.0, 150 mM NaCl and 1 mM TCEP). The bound PDIa was eluted with 250 mM imidazole in the same buffer. Recombinant PDIa was further purified using gel filtration Superdex 100 column (GE Life Sciences) in a buffer containing 20 mM Tris-HCl, pH 8.0, 150 mM NaCl, 1 mM TCEP. The fractions containing PDIa were concentrated, flash frozen, and stored at −80° C. Protein concentration was determined using absorbance at 280 nm with molar extinction coefficient (E) 19940 M⁻¹ cm⁻¹ (for reduced PDIa with N-terminal His₆ tag as calculated from amino acid sequence by ExPASy ProtParam). PDIa purity was verified by SDS-PAGE as more than 98% pure.

For NMR studies, uniformly ¹⁵N-labeled PDIa protein with an N-terminal His₆ tag was prepared. The PDIa construct was transformed into Escherichia coli BL21-Gold (DE3) competent cells (Agilent Technologies). Cells were grown at 37° C. in 1 L of M9 minimal medium supplemented with 2 mM MgSO₄, 0.1 mM CaCl₂, 100 μg/ml ampicillin, 22.2 mM glucose, metals 44 solution, 30 mg nicotinic acid, 3 mg p-aminobenzoic acid, 0.3 mg biotin, 0.5 mg thiamine hydrochloride, and 0.6 g ¹⁵NH₄Cl as the sole nitrogen source. When OD_{600 nm} reached 0.9, the temperature was reduced to 20° C. and protein expression was induced with 0.5 mM IPTG for overnight. Protein was purified as described above except the histidine tag was removed after size exclusion chromatography. Thrombin was added at 5 U/mg protein to cleave the N-terminal His₆ tag. The reaction was allowed to proceed overnight at 4° C. The next day, protein solution was passed over Ni Sepharose 6 Fast Flow beads (GE Life Sciences) equilibrated with PDI Suspension Buffer and flow-through containing the ¹⁵N-labeled PDIa protein without histidine tag was concentrated, and incubated with 5 mM TCEP overnight with gentle shaking at 4° C. The next day, the protein was dialyzed into MilliQ water, flash frozen and stored at −80° C. ¹⁵N-labeled PDIa purity was verified by SDS-PAGE as more than 98% pure.

### Isothermal Titration Calorimetry (ITC)

All ITC experiments were carried out at 25° C. on MicoCal Auto-ITC₂₀₀ system (GE Healthcare). Reduced PDIa was dialyzed into ITC buffer (20 mM sodium phosphate buffer pH 7.8) and loaded into a sample cell at 40 μM concentration. The compound solution was loaded into a syringe at 400 μM in the same ITC buffer with a final DMSO concentration at 0.4% (v/v). ITC titration experiments were carried out at 25° C. with 19 injections, 2 μl per injection, and 180 seconds between each injection. The reference cell power was set to 5 μcal/sec. A control experiment was performed for each compound, where each compound was titrated into buffer to account for heat released due to dilution. This background was subtracted from test data before a final dissociation constant was obtained. Data were analyzed using a one-site binding model in Origin 7.1 software. The dissociation constant, K_{d}, was calculated according to equation K_{d}=1/Kₐ. Gibbs free energy, ΔG, was calculated from equation ΔG=ΔH−TΔS. All other parameters, Kₐ, n, ΔH, ΔS, were determined directly from the titration data.

For ITC experiments with 16F16 pre-treatment, 40 μM PDIa (after dialysis into ITC buffer) was treated with 200 μM 16F16 for 12-15 hours at 4° C. Next day the whole solution was loaded into the sample cell. LOC14 at 400 μM in ITC buffer was loaded into syringe and titrated into PDIa+16F16 loaded cell. For control experiments, ITC buffer with 200 μM 16F16 was used in the cell, while 400 μM LOC14 in ITC buffer was used in the syringe.

### Size-Exclusion Buffer Dialysis Experiments for Reversibility of Binding

Prior to dialysis, fluorescence emission spectra were recorded from 315 nm-550 nm wavelength with excitation at 280 nm on a Tecan Infinite 200 microplate reader. Fluorescence readings were carried out in a 384-well low volume, black bottom plate. Each well contained 40 μl of either LOC14 (300 μM), PDIa (20 μM), or PDIa (20 μM) treated with LOC14 (300 μM) overnight. All samples were dissolved in buffer B (20 mM sodium phosphate buffer pH 7.8). After recording the initial fluorescence spectra, samples were then transferred to Amicon Ultra 10 kDa size exclusion filter spin columns for dialysis. 400 μl of buffer B was added to the spin column, the samples were centrifuged for 8 minutes on a table top microcentrifuge (12,000 rpm, 4° C.), and afterwards the flow-through was transferred to a new tube. This step was repeated three more times with fresh buffer B. 40 μl of the collected samples from the flow-through and the spin-column chamber were transferred to a new 384-well plate and the emission spectra recorded as described above.

### NMR Spectroscopy

The residue numbering in all HSQC spectra are based on the sequence of the mature PDI protein (SEQ ID NO: 3) i.e., residue 1 of the mature PDI corresponds to residue 18 in the full length PDI. The first 17 amino acids in full length PDI are the signal sequence that is processed out to generate the mature PDI.

The ¹H-¹⁵N HSQC spectra were performed on Bruker Avance III 500 Ascend (500 MHz) spectrometers at 300 K. The uniformly ¹⁵N-labeled PDIa was dissolved at 50 μM or 100 μM in 90% H₂O/10% D₂O (v/v), pH 5.1. The ¹H carrier frequency was positioned at the water resonance. The ¹⁵N carrier frequency was positioned at 115 ppm. The spectral width in the ¹H dimension was 7500 Hz and the width in ω₁ (¹⁵N) dimension was 1824.6 Hz. Suppression of water signal was accomplished using the WATERGATE sequence. Heteronuclear decoupling was accomplished using GARP decoupling scheme.

The 3D NMR experiments were performed on a Bruker Avance 500 MHz spectrometer equipped with a 5 mm TXI cryogenic probe. The ¹⁵N-NOESY-HSQC and ¹⁵N-TOCSY-HSQC spectra were recorded at 300 K on the uniformly ¹⁵N-labeled PDIa that was dissolved at 500 μM in 90% H₂O/10% D₂O (v/v), pH 5.1. The proton carrier frequency was positioned at the water resonance. The ¹⁵N carrier frequency was positioned at 118 ppm. The spectral width in the ¹H dimension was 7501.9 Hz and the width in ω₂ (¹⁵N) dimension was 2027.3 Hz. Suppression of water signal was accomplished using the WATERGATE sequence. The ¹⁵N-NOESY-HSQC was recorded using the mixing time of 150 msec. The ¹⁵N-TOCSY-HSQC was recorded using the mixing time of 60 msec (Kemmink et al., 1995). All the data was processed and analyzed using TopSpin 3.1 (Bruker). The assignments were performed using Sparky (T. D. Goddard and D. G. Kneller, UCSF). The mean chemical shift difference for ¹H and ¹⁵N (Δδ_{NH}) was calculated using formula (Williamson, 2013):

$$Δ {δ}_{NH} = \frac{\sqrt{{( Δ δ {H}_{N} )}^{2} + {( ( \frac{Δ δ H}{10} ) )}^{2}}}{2}$$

### Mouse Microsome Stability Assay

Test compound (0.5 μM) was incubated at 37° C. for up to 45 minutes in 100 mM of potassium phosphate buffer (pH 7.4) containing microsomal protein (0.5 mg/mL) and an NADPH generating system (0.34 mg/mL β-nicotinamide adenine dinucleotide phosphate (NADP), 1.56 mg/mL glucose-6-phosphate, and 1.2 units/mL glucose-6-phosphate dehydrogenase). At 0, 5, 15, 30 and 45 minute intervals, an aliquot was taken and quenched with acetonitrile (ACN) containing internal standard. No-cofactor controls at 45 minutes were prepared. Following completion of the experimentation, the samples were analyzed by LC-MS/MS. The half-life (t_{1/2}) was calculated using the following equation: t_{1/2}=0.693/k, where k is the elimination rate constant of test compounds obtained by fitting the data to the equation: C=initial×exp (−k×t). Intrinsic clearance (CLᵢₙₜ) was calculated as liver clearance from the half-life using the following equation: CLᵢₙₜ=k×(ml incubation/0.5 mg protein)×(52.5 mg protein/g liver). Results were reported as peak area ratios of analyte to internal standard. The intrinsic clearance (CLᵢₙₜ) was determined from the first order elimination constant by non-linear regression.

### In Vitro Drug Metabolism Studies: Mouse Plasma Stability Assay

Test compound (1 μM) was incubated at 37° C. for up to 120 minutes in mouse plasma. At 0, 15, 30, 60, and 120 minute intervals an 100 μL aliquot was taken and quenched with 200 μL acetonitrile (ACN) containing internal standard. Following completion of the experimentation, the samples were analyzed by LC-MS/MS. The half-life (t_{1/2}) was calculated using the following equation: t_{1/2}=0.693/k, where, k is the elimination rate constant of test compounds obtained by fitting the data to the equation: C=initial×exp (−k×t). Results were reported as peak area ratios of analyte to internal standard.

In Vitro Drug Metabolism Studies: Mouse plasma protein binding assay

A test compound at the concentration of 2000 ng/mL in plasma was added into the sample chamber, and a dialysis buffer phosphate-buffered saline (PBS) was added into the buffer chamber, covering the unit with sealing tape and incubating for 4 hours at 37° C. at approximately 100 rpm on an orbital shaker. The incubation samples were taken from both plasma and buffer chamber at the end of the incubation, and then samples were analyzed by LC-MS/MS. Protein binding and free fraction percentage were determined using peak area ratio of analyte to internal standard. Fraction bound percent was calculated as: % Bound=100*(Cₚₗₐₛₘₐ−C_{PBS})/Cₚₗₐₛₘₐ. The fraction recovered percent was calculated as: % Recovery=(V_{PBS}*C_{PBS}+Vₚₗₐₛₘₐ*Cₚₗₐₛₘₐ)/(Vₚₗₐₛₘₐ*Cₛₚᵢₖₑ) where V_{PBS} is Volume of PBS, Vₚₗₐₛₘₐ is Volume of Plasma, C_{PBS} is Drug concentration in PBS (Analyte/IS peak area ratio), Cₚₗₐₛₘₐ is Drug concentration in plasma (Analyte/IS peak area ratio), Cₛₚᵢₖₑ is Drug concentration in spiked plasma (Analyte/IS peak area ratio).

### In Vitro Drug Metabolism Studies

The microsome stability assay, plasma stability assay, and plasma protein binding assay were each performed by Alliance Pharma, Inc. (Malvern, Pa.).

Liquid Chromatography-Mass spectrometry

Proteins were separated by one-dimensional SDS-PAGE electrophoresis and digested with trypsin as described previously (Cardinale et al., 2008). Peptides were separated with a NanoAcquity UPLC as described previously (Yang et al., 2014) except that Solvent B was increased in a 30 minute linear gradient between 5 and 40% and post-gradient cycled to 95% B for 7 min, followed by post-run equilibration at 5% B.

Spectra were recorded in sensitivity positive ion mode with a Synapt G2 quadrupole-time-of-flight HDMS mass spectrometer (Waters Corp). Spectra were acquired for the first 59 minutes of the chromatographic run. Source settings were capillary voltage (3.2 kV), extraction cone (4 V), sampling cone (30 V), and source temperature of 80° C. The cone gas N₂ flow was 30 L/hour. Analyzer settings included quadrupole profile set at manual with mass 1 as 400 (dwell time 25%, ramp time 25%), mass 2 as 500 (dwell time 25%, ramp time 25%) and mass 3 as 600. A reference sprayer was operated at 500 nL/minute to produce a lockmass spectrum with Glu-1-Fibrinopeptide B (EGVNDNEEGFFSAR) (m/z 785.8426) leucine enkephalin (YGGFL) at m/z 556.2771 every 30 s.

Data were collected by data-dependent acquisition with a scan time of 0.25 seconds. A survey scan was conducted over the range of 300 to 2000 Da. Acquisition was performed in sensitivity mode and switched when individual ion counts exceeded 1000 count/second. MS/MS spectra were acquired over the range of 50 to 2000 m/z. A maximum of the five most intense ions were selected from a single MS survey scan. Return to MS survey scan was triggered when the intensity exceeded 60,000 counts/second or 3 seconds had elapsed. Charge state peak detection was enabled for +2, +3, and +4. Collision energy in the trap was ramped from 12 to 20 volts for low mass (300 Da) and 40 to 60 V for high mass (2000 Da).

Raw spectrum processing was performed with the PLGS software (Vers. 2.5, RC9). The electrospray survey was calibrated to a lock mass of Glu-1-Fibrinopeptide B at 785.8426 m/z, averaging three scans with a tolerance of 0.1 Da, adaptive background subtraction with slow algorithm deisotoping function with 30 iterations and a 3% threshold. MS/MS spectra were calibrated to singly-charged leucine enkephalin at m/z 556.2771 with the same settings as for the survey scan. Spectra were processed and exported as .pkl files that were then imported into the Mascot database search program (Vers. 2.3.02) (Matrix Science, London, UK.). Observed masses were searched with Mascot against the full NCBI nr protein database of Jan. 4, 2012 (16,826,875 sequences; 5,780,204,515 residues). In addition, a custom database was used with sequence of the protein disulfide isomerase construct used for the reaction. Decoy search was enabled and no taxonomic restriction and up to one missed trypsin cleavage with no fixed modifications. Charge states of +1, +2, or +3 were considered. Monoisotopic mass tolerance for precursor peptides was 10 ppm, and for products, 0.02 Da. Variable modifications included carbamidomethyl (C), oxidation (M) and a custom modification of C representing inhibitor 16F16 with a monoisotopic mass shift of 284.1160 Da and an elemental composition C (16) H (16) N (2) O (3).

All raw data files and DDA .pkl files were deposited in a public repository at www.chorusproject.org.

### HD Brain Slice Assay

Brain slice explants were prepared and transfected as previously described (Reinhart et al., 2011). Briefly, brains were taken from postnatal day 10 CD Sprague-Dawley rat pups and cut into 250 μm coronal slices on a vibratome (Vibratome Co., St. Louis, Mo.). Brain slices containing striatum and cortex were then placed in individual wells of 12-well plates atop culture medium set in 0.5% agarose and maintained at 32° C. under 5% CO₂. Co-transfection with YFP and Htt exon-1 containing a 73 polyglutamine repeat was done using a biolistic device (Bio-Rad Helios Gene Gun, Hercules, Calif.). Positive controls were transfected with YFP only or treated with a combination of 50 μM KW-6002 (istradefylline) and 50 μM SP600125. Negative control brain slices were treated with 0.1% DMSO carrier only. Striatal medium spiny neurons (MSNs) expressing YFP were visualized under fluorescence microscopy and identified based on their location within brain slices and their characteristic morphology. MSNs exhibiting normal-sized cell bodies, and even and continuous expression of YFP in at least 2 discernible primary dendrites at least 2 cell body diameters long were scored as healthy.

### Example 2

### Compound Synthesis

All commercial reagents were used without further purification. All solvents used were reagent or HPLC grade. All reactions were carried out in flame-dried glassware under a nitrogen atmosphere. Chemical yields refer to isolated, spectroscopically pure compounds. Proton (¹H) and carbon (¹³C) NMR spectra were recorded on a Bruker Avance III 400 or 500 MHz spectrometer at ambient temperature. Chemical shifts were recorded in parts per million relative to residual solvent CDCl₃ (1H, 7.26 ppm; ¹³C, 77.16 ppm). Multiplicities were reported as follows: s=singlet, d=doublet, t=triplet, q=quartet, m=multiplet, comp m=complex multiplet, td=triplet of doublets.

### 2-((4-(cyclopropanecarbonyl)piperazin-1-yl)methyl)benzo[d]isothiazol-3(2H)-one (LOC14)

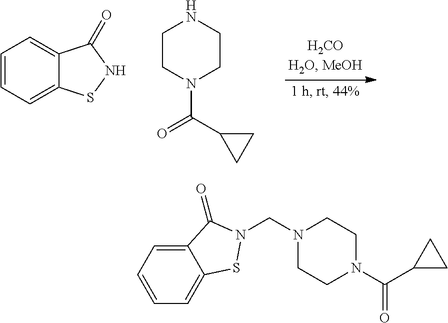

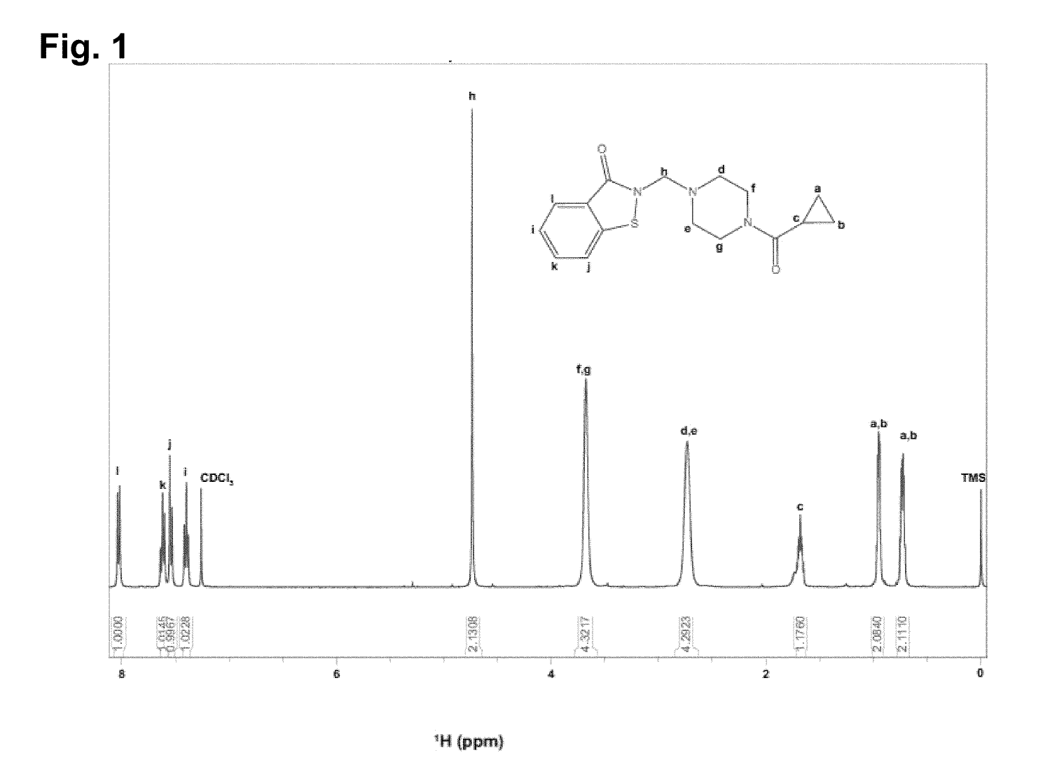

Methanol (1 mL) and a 38% solution of formaldehyde in water (75 μL 1.2 eq.) were combined and stirred at room temperature. 1,2-Benzisothiazol-3(2H)-one (98 mg, 1 eq.) and 1-(cyclopropylcarbonyl)piperazine (92 μL, 1 eq.) were then added and the solution was allowed to stir at room temperature until white solids precipitated, about one hour. These solids were collected via vacuum filtration and recrystallized from ethyl acetate and hexanes providing LOC14 (91 mg, 44%) as a white powder. ¹H NMR (400 MHz, CDCl₃) δ 8.037 (d, J=8 Hz, 1H), 7.63 (t, J=7.2 Hz, 1H), 7.55 (d, J=8 Hz, 1H), 7.397 (t, J=7.2 Hz, 1H), 4.73 (s, 1H), 3.67 (bs, 4H), 2.73 (bs, 4H), 1.67 (m, 1H), 0.96 (m, 2H), 0.74 (m, 2H); ¹³C NMR (500 MHz, CDCl₃) δ 172.0, 166.3, 141.1, 132.2, 126.9, 125.6, 124.7, 120.4, 65.7, 50.7, 50.2, 45.4, 42.1, 11.0, 7.5. LRMS (APCI+): calculated for (C₁₆H₁₉N₃O₂S) 317.4 g/mol, found m/z (relative intensity %): 317.07 (M⁺, 1%), 309.10 (1%), 302.90 (1%), 269.16 (4%), 184.15 (10%), 167.19 (4%), 155.18 (100%), 152.08 (100%), 122.12 (6%). This mass fragmentation pattern was consistent with the properties reported for Mannich bases (Idhayadhulla et al., 2011; Holla et al., 1998; Fuchslueger et al., 1999). The identity of LOC14 as a single compound under non-ionizing conditions was verified by thin layer chromatography in 10% MeOH/90% Dichloromethane, showing a single spot with an Rf of 0.5 upon iodine visualization and by NMR (see FIG. 1).

### 2-((4-(cyclopropanecarbonyl)piperazin-1-yl)methyl)benzo[d]isoxazol-3(2H)-one (Oxy-LOC14)

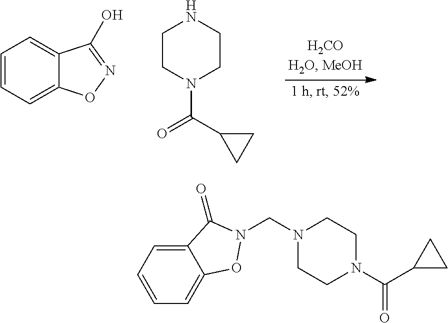

Methanol (1 mL) and a 38% solution of formaldehyde in water (75 μL 1.2 eq.) were combined and stirred at room temperature. 3-hydroxybenzisoxazole (87 mg, 1 eq.) and 1-(cyclopropylcarbonyl)piperazine (92 μL, 1 eq.) were then added and the solution was allowed to stir at room temperature for one hour. Precipitation of a white solid was induced by sonication. These solids were collected via vacuum filtration and recrystallized from ethyl acetate and hexanes providing Oxy-LOC14 (101 mg, 52%) as a white powder. ¹H NMR (400 MHz, CDCl₃) δ 7.83 (d, J=8 Hz, 1H), 7.64 (t, J=6 Hz, 1H), 7.30 (t, J=8 Hz, 1H), 7.23 (d, J=8.4 Hz, 1H), 4.93 (s, 2H), 3.68 (bs, 4H), 2.78 (bs, 4H), 1.67 (m, 1H), 0.95 (m, 2H), 0.74 (m, 2H); ¹³C NMR (500 MHz, CDCl₃) δ 171.9, 163.4, 160.3, 133.69, 124.5, 123.6, 115.94, 110.0, 67.136, 50.5, 50.0, 45.3, 42.0, 10.8, 7.42.

### tert-butyl 4-((3-oxobenzo[d]isothiazol-2(3H)-yl)methyl)piperazine-1-carboxylate

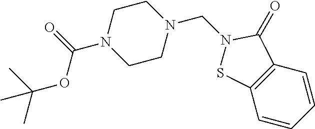 [SMILES: CC(C)(C)OC(=O)N1CCN(Cn2sc3ccccc3c2=O)CC1]

To a solution of 37% aq. formaldehyde (75 uL) in MeOH (1 mL) was added benzoisothiazol-3-one (98 mg, 0.65 mmol) and tert-butyl piperazine-1-carboxylate (121 mg, 0.65 mmol). After 18 hours the solution was cooled to 0° C. and water was added. The resulting precipitate was filtered, washed with water and dried in vacuo to give the title compound as a white powder (157 mg, 69% yield). ¹H NMR (500 MHz, CDCl₃) δ 8.03 (d, 1H), 7.62 (t, 1H), 7.54 (d, 1H), 7.40 (t, 1H), 4.71 (s, 2H), 3.45 (bds, 4H), 2.67 (bds, 4H), 1.43 (s, 9H). ¹³C NMR (500 MHz, CDCl₃) 166.5, 154.9, 141.4, 132.4 127.2, 125.8, 125.1, 120.7, 80.1, 66.1, 50.6, 28.7.

### 2,2′-(piperazine-1,4-diylbis(methylene))bis(benzo[d]isothiazol-3(2H)-one)

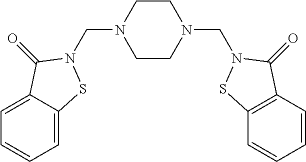 [SMILES: O=c1c2ccccc2sn1CN1CCN(Cn2sc3ccccc3c2=O)CC1]

To a solution of 37% aq. formaldehyde (1 mL) in EtOH (19.5 mL) was added benzoisothiazol-3-one (98 mg, 0.65 mmol) and piperazine (56 mg, 0.65 mmol). The reaction mixture was heated at 80° C. for 12 hours then allowed to cool to room temperature. After 3 days the white precipitate was collected by centrifugation and dried in vacuo to give the title compound as a white powder (75 mg, 28% yield). ¹H NMR (400 MHz, CDCl₃) δ 8.03 (d, 2H), 7.60 (t, 2H), 7.52 (d, 2H), 7.39 (t, 2H), 4.69 (s, 4H), 2.77 (bds, 8H). ¹³C NMR (500 MHz, CDCl₃) 166.5, 141.5, 132.3, 127.1, 125.7, 125.2, 120.6, 66.0, 50.5.

### 5-chloro-2-((4-(cyclopropanecarbonyl)piperazin-1-yl)methyl)benzo[d]isothiazol-3(2H)-one

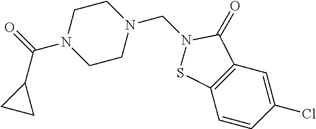 [SMILES: O=C(C1CC1)N1CCN(Cn2sc3ccc(Cl)cc3c2=O)CC1]

To a solution of 37% aq. formaldehyde (15 uL) in MeOH (1 mL) was added 5-chlorobenzoisothiazol-3-one (25 mg, 0.13 mmol) and cyclopropyl(piperazin-1-yl)methanone (20 mg, 0.13 mmol). The resulting suspension was stirred for several days, cooled to 0° C. and resulting precipitate was filtered to give the title compound as a white powder (35.6 mg, 78% yield). ¹H NMR (400 MHz, CDCl₃) δ 8.00 (d, 1H), 7.59 (dd, 1H), 7.48 (d, 2H), 4.72 (s, 2H), 3.68 (bds, 4H), 2.73 (bds, 4H), 1.68 (m, 1H), 0.96 (m, 1H), 0.74 (m, 1H).

### 2,2′-methylenebis(benzo[d]isothiazol-3(2H)-one)

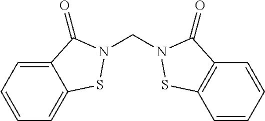 [SMILES: O=c1c2ccccc2sn1Cn1sc2ccccc2c1=O]

To a solution of 37% aq. formaldehyde (24 uL) in MeOH (1 mL) was added benzoisothiazol-3-one (32 mg, 0.21 mmol). After several days the reaction mixture was cooled to 0° C. and the crystalline material collected to give the title compound. ¹H NMR (500 MHz, CDCl₃) δ 8.00 (d, 2H), 7.60 (t, 2H), 7.51 (d, 2H), 7.37 (t, 2H), 5.37 (s, 4H).

### 4-((benzo[d]isothiazol-3-yloxy)methyl)piperidine-1-carboxylate

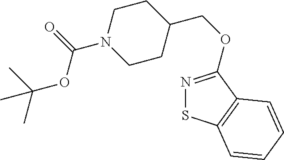 [SMILES: CC(C)(C)OC(=O)N1CCC(COc2nsc3ccccc23)CC1]

To benzoisothiazol-3-one (27 mg, 0.18 mmol) and 60% NaH in oil (9 mg, 0.22 mmol) was added anhydrous DMF. After gas evolution ceased, tert-butyl 4-(bromomethyl)piperidine-1-carboxylate (50 mg, 0.18 mmol) was added. After 72 h, the solution was diluted with EtOAc and washed with water and aq. NaHCO₃. Concentration in vacuo gave a colorless oil (59 mg). Silica gel chromatography (hexane:EtOAc) gave the title compound as a solid (40 mg, 64% yield). 1H NMR (400 MHz, CDCl₃) δ 7.95 (d, 1H), 7.77 (d, 2H), 7.52 (t, 1H), 7.38 (t, 1H), 4.40 (d, 2H), 4.16 (m, 2H), 2.77 (m, 2H), 2.09 (m, 1H), 1.85 (m, 2H), 1.47 (s, 9H), 1.33 (m, 2H). ¹³C NMR (400 MHz, CDCl₃) 163.4, 155.2, 152.0, 129.0, 125.7, 124.7, 123.3, 120.5, 79.8, 73.0, 43.8, 36.3, 29.1, 28.8.

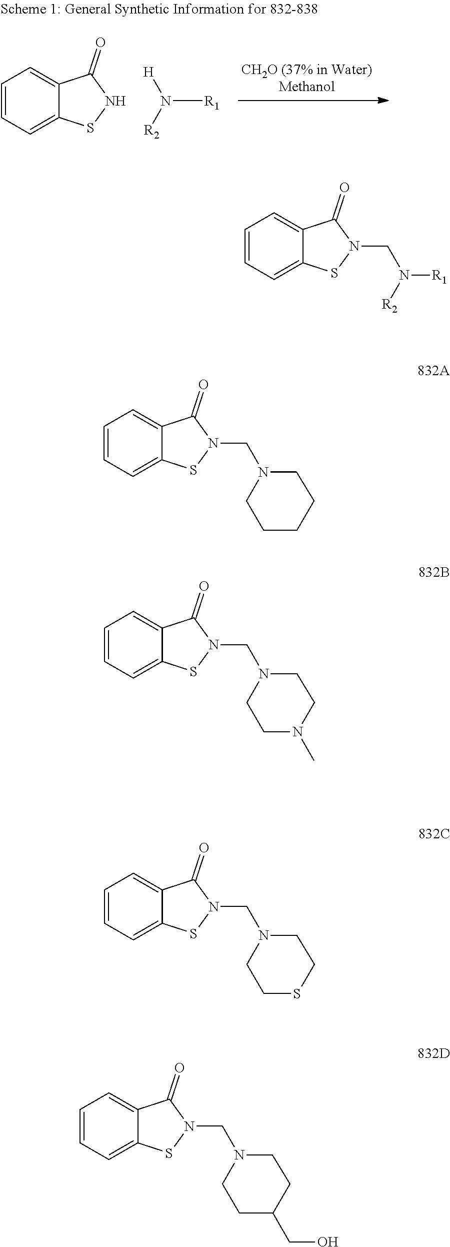

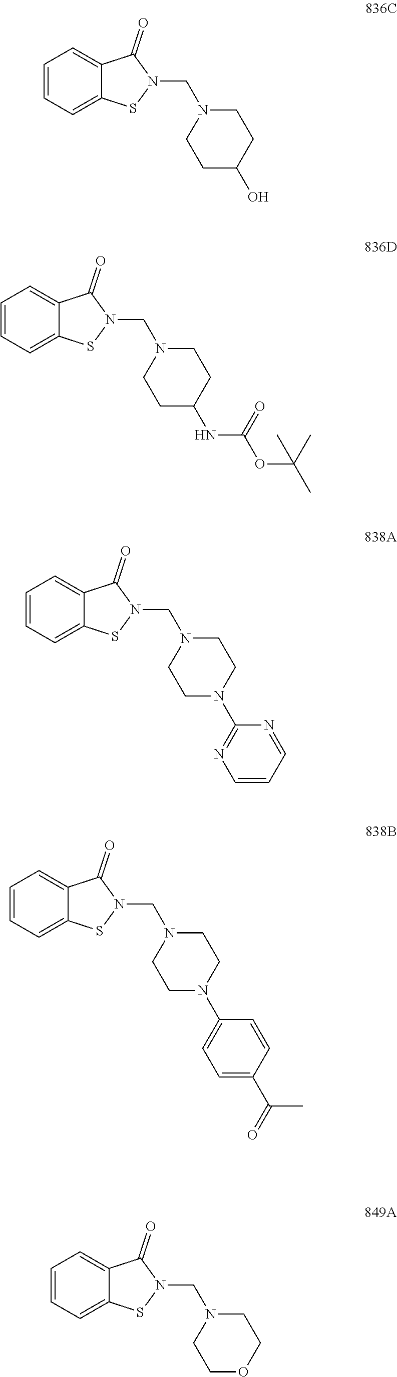 [SMILES: O=c1c2ccccc2sn1CN1CCC(O)CC1 ; CC(C)(C)OC(=O)NC1CCN(Cn2sc3ccccc3c2=O)CC1 ; O=c1c2ccccc2sn1CN1CCN(c2ncccn2)CC1 ; CC(=O)c1ccc(N2CCN(Cn3sc4ccccc4c3=O)CC2)cc1 ; O=c1c2ccccc2sn1CN1CCOCC1]

### General Procedure a (Used for all Compounds Except 849A)

0.992 mmol of 1,2,-benzoisothiazol-1-one were combined with 1 mL of methanol and 116 uL (1.19 mmol; 1.2 eq) of a 37% solution of formaldehyde in water. Suspended solids were dissolved into solution with brief heating. To this solution was added 0.992 mmol of the corresponding secondary amine. The resulting solution was let stir at room temperature overnight or until solids formed. Any residual solvent was removed under reduced pressure, and solids were induced to precipitate from a mixture of ethyl acetate and hexanes.

### General Procedure B (849A)

0.992 mmol of 1,2,-benzoisothiazol-1-one were combined with 1 mL of methanol and 116 uL (1.19 mmol; 1.2 eq) of a 37% solution of formaldehyde in water. Suspended solids were dissolved into solution with brief heating. To this solution was added 0.992 mmol of the corresponding secondary amine. The solution was left to stir overnight. Solvent was removed under reduced pressure, leaving an oil. This oil was purified on a silica column by running a gradient of 0410% methanol in dichloromethane.

### Spectral Data

8-32A ¹H NMR (400 MHz, Chloroform-d) b 8.03 (dt, J=7.9, 1.0 Hz, 1H), 7.60 (ddd, J=8.2, 7.0, 1.3 Hz, 1H), 7.53 (dt, J=8.1, 1.0 Hz, 1H), 7.38 (ddd, J=8.0, 7.0, 1.1 Hz, 1H), 4.67 (s, 2H), 2.88-2.41 (m, 4H), 1.60 (p, J=5.7 Hz, 8H), 1.44 (tq, J=8.5, 5.4, 4.4 Hz, 2H).

8-32B ¹H NMR (300 MHz, Chloroform-d) b 8.02 (dt, J=7.9, 1.0 Hz, 1H), 7.60 (ddd, J=8.2, 7.0, 1.3 Hz, 1H), 7.53 (dt, J=8.1, 1.0 Hz, 1H), 7.38 (ddd, J=8.0, 6.9, 1.2 Hz, 1H), 4.69 (s, 2H), 2.76 (t, J=4.9 Hz, 4H), 2.45 (s, 4H), 2.28 (s, 3H).

8-32C ¹H NMR (400 MHz, Chloroform-d) b 8.03 (dt, J=7.9, 1.0 Hz, 1H), 7.62 (ddd, J=8.2, 7.1, 1.3 Hz, 1H), 7.54 (dt, J=8.1, 0.9 Hz, 1H), 7.40 (ddd, J=8.0, 7.0, 1.1 Hz, 1H), 4.70 (s, 2H), 3.13-2.94 (m, 4H), 2.75-2.60 (m, 4H).

8-32D ¹H NMR (400 MHz, Chloroform-d) δ 8.03 (dt, J=7.9, 1.3, 0.8 Hz, 1H), 7.60 (ddd, J=8.2, 7.0, 1.3 Hz, 1H), 7.54 (dt, J=8.0, 1.0 Hz, 1H), 7.39 (ddd, J=8.0, 7.0, 1.1 Hz, 1H), 4.69 (s, 2H), 3.50 (d, J=6.3 Hz, 2H), 3.11-2.99 (m, 2H), 2.40 (td, J=11.7, 2.6 Hz, 2H), 1.75 (d, J=12.2 Hz, 2H), 1.53-1.43 (m, 1H), 1.30 (qd, J=12.1, 4.0 Hz, 2H).

8-36C ¹H NMR (300 MHz, DMSO-d₆) δ 7.95 (dt, J=8.1, 0.9 Hz, 1H), 7.86 (dt, J=7.7, 1.0 Hz, 1H), 7.69 (ddd, J=8.2, 7.1, 1.3 Hz, 1H), 7.43 (ddd, J=8.0, 7.1, 1.0 Hz, 1H), 4.61 (s, 2H), 4.57 (d, J=4.2 Hz, 1H), 3.52-3.36 (m, 1H), 2.82 (dd, J=11.0, 5.3 Hz, 2H), 2.41-2.29 (m, 2H), 1.71 (dd, J=13.1, 4.1 Hz, 2H), 1.50-1.30 (m, 2H).

8-36D ¹H NMR (300 MHz, Chloroform-d) δ 8.03 (dt, J=7.8, 1.0 Hz, 1H), 7.61 (ddd, J=8.2, 6.9, 1.3 Hz, 1H), 7.54 (dt, J=8.1, 1.0 Hz, 1H), 7.39 (ddd, J=8.0, 6.9, 1.2 Hz, 1H), 4.67 (s, 2H), 4.43 (s, 1H), 3.45 (s, 1H), 2.95 (dd, J=10.2, 5.9 Hz, 2H), 2.49 (td, J=11.5, 2.7 Hz, 2H), 1.99-1.85 (m, 2H), 1.68 (s, 2H), 1.42 (s, 10H).

8-38A ¹H NMR (400 MHz, Chloroform-d) δ 8.28 (d, J=4.7 Hz, 2H), 8.03 (ddd, J=7.9, 1.3, 0.8 Hz, 1H), 7.61 (ddd, J=8.2, 7.0, 1.2 Hz, 1H), 7.53 (dt, J=8.1, 0.9 Hz, 1H), 7.39 (ddd, J=8.1, 7.1, 1.1 Hz, 1H), 6.46 (t, J=4.7 Hz, 1H), 4.75 (s, 2H), 3.93-3.79 (m, 4H), 2.84-2.73 (m, 4H).

8-38B ¹H NMR (400 MHz, Chloroform-d) δ 8.06-8.02 (m, 1H), 7.87-7.83 (m, 2H), 7.62 (ddd, J=8.3, 7.1, 1.3 Hz, 1H), 7.54 (dt, J=8.1, 0.9 Hz, 1H), 7.40 (ddd, J=8.1, 7.1, 1.0 Hz, 1H), 6.87-6.82 (m, 2H), 4.76 (s, 2H), 3.41-3.34 (m, 4H), 2.90-2.83 (m, 4H), 2.50 (s, 3H).

8-49A ¹H NMR (400 MHz, Chloroform-d) b 8.04-8.00 (m, 1H), 7.59 (ddd, J=8.2, 6.9, 1.3 Hz, 1H), 7.54 (dt, J=8.1, 1.0 Hz, 1H), 7.37 (ddd, J=8.0, 6.9, 1.3 Hz, 1H), 4.66 (s, 2H), 3.73-3.66 (m, 5H), 2.73-2.66 (m, 5H).

### 2-benzylbenzo[d]isothiazol-3(2H)-one

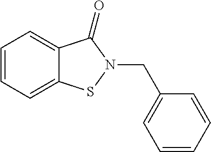 [SMILES: O=c1c2ccccc2sn1Cc1ccccc1]

1,2-benzisothiazol-3(2H)-one (1.0 eq, 2.5 mmol, 378 mg), Benzylbromide (2.0 eq, 5.0 mmol, 855 mg, 595 μL) were dissolved in tetrahydrofuran (5 mL), potassium carbonate (2.5 eq, 6.25 mmol, 864 mg) was added and the mixture was stirred overnight. The solvent was evaporated and the residue partitioned between water and ethyl acetate. The layers were separated and the aqueous layer was extracted with ethyl acetate (2×5.0 mL). The combined organic layer was dried with magnesium sulfate, filtered and the solvent evaporated. The crude product was purified by column chromatography on silica (0-60% ethyl acetate/n-hexanes) to give 2-benzylbenzo[d]isothiazol-3(2H)-one (415 mg, 69% yield) as a colorless oil that crystallized upon standing. The spectroscopic data matched those reported in literature. (Correa et al., 2006)

### 3-oxo-N-phenylbenzo[d]isothiazole-2(3H)-carboxamide

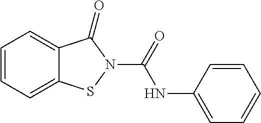 [SMILES: O=C(Nc1ccccc1)n1sc2ccccc2c1=O]

Aniline (1.0 eq, 0.5 mmol, 47 mg, 46 μL) was dissolved in dichloromethane (5 mL) and triethylamine was added (10.0 eq, 5.0 mmol, 506 mg, 693 μL). The resulting solution was added dropwise to a solution of triphosgene (1.0 eq, 0.5 mmol, 148 mg) in dichloromethane (5 mL) and the reaction mixture was stirred for 5 minutes at room temperature. The reaction mixture was added dropwise to a solution of 1,2-benzisothiazol-3(2H)-one (1.0 eq, 0.5 mmol, 76 mg) in tetrahydrofuran (2.0 mL). After stirring at room temperature overnight, the solvent was evaporated. Upon addition of acetone (3 mL) and water (3.5 mL) the product precipitated and was filtered off. After drying overnight 3-oxo-N-phenylbenzo[d]isothiazole-2(3H)-carboxamide (103 mg, 76% yield) was isolated as a pale yellow solid. The spectroscopic data matched those reported in literature. (Liu et al., 2013)

The other listed compounds disclosed herein are synthesized in a similar fashion or obtained as described herein.

### Example 3

### High Throughput Screen Identifies Small Molecule Inhibitors of PDI

Using a phenotypic high throughput screening approach, a small-molecule, neuroprotective compound 16F16 was previously identified (Hoffstrom et al., 2010). The alpha-chloro ketone moiety on this molecule made it likely an irreversible inhibitor and this property aided subsequent pull-down experiments that identified PDI as its target. Modulation of PDI by 16F16 was beneficial in an in vitro model of AD using rat corticostriatal brain slices expressing amyloid precursor protein, which is processed in situ to Aβ peptides centrally implicated in the amyloid-cascade hypothesis of AD. However, the reactive alpha-chloro ketone group and irreversible inhibition of PDI by 16F16 prompted us to search for compounds that were reversible inhibitors with improved properties that were suitable for in vivo studies.

To identify neuroprotective PDI modulators with attractive pharmaceutical properties, a library with lead-optimized compounds was assembled (Cantley et al., 2014; Dixon et al., 2012). Initially, a database of 3,372,615 commercially available small molecules (from Asinex, Life Chemicals, Enamine, TimTec, InterBioScreen and Chembridge suppliers) was compiled and stringently filtered computationally. The compounds were filtered to adhere to the Lipinski rule of five (Lipinski et al., 2001) and a molecular weight minimum of 235; compounds with noxious or reactive properties and water solubility less than 0.5 mM were eliminated. Strong emphasis was placed on compounds suitable for lead development; therefore potential nonspecific binders that might be acting as cationic and nonionic detergents were removed. Scaffolds with more than five rotatable bonds and a topological polar surface area larger than 70 Å² were eliminated to improve the likelihood of blood-brain barrier penetration. As a final step, the compounds were clustered based on their Tanimoto coefficient and only a diverse subset was purchased. The final lead optimized compound (LOC) library contained 9,719 unique small molecules.

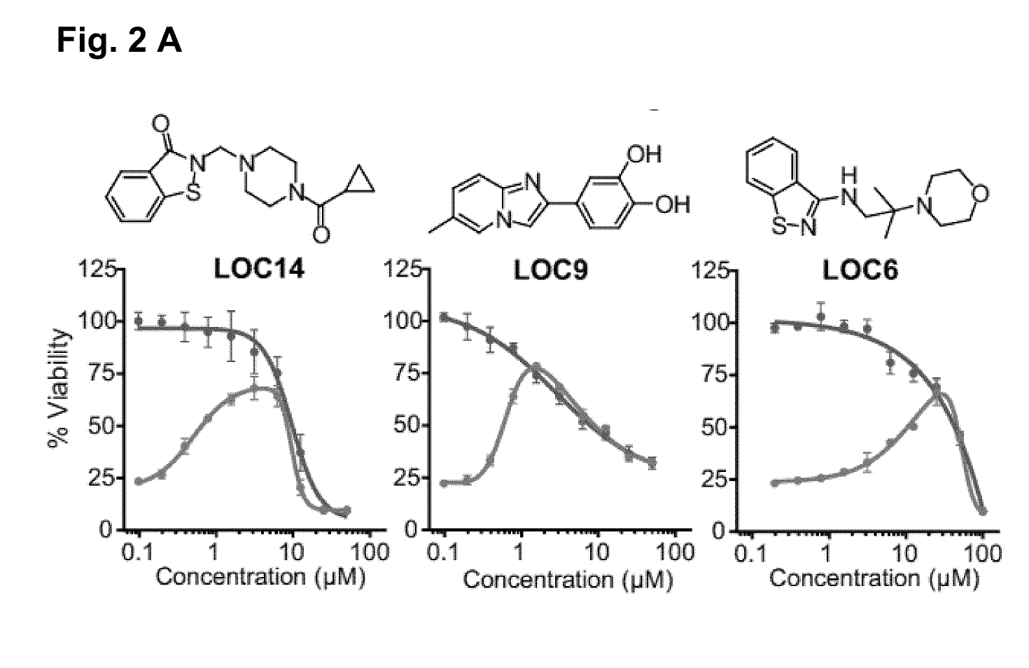

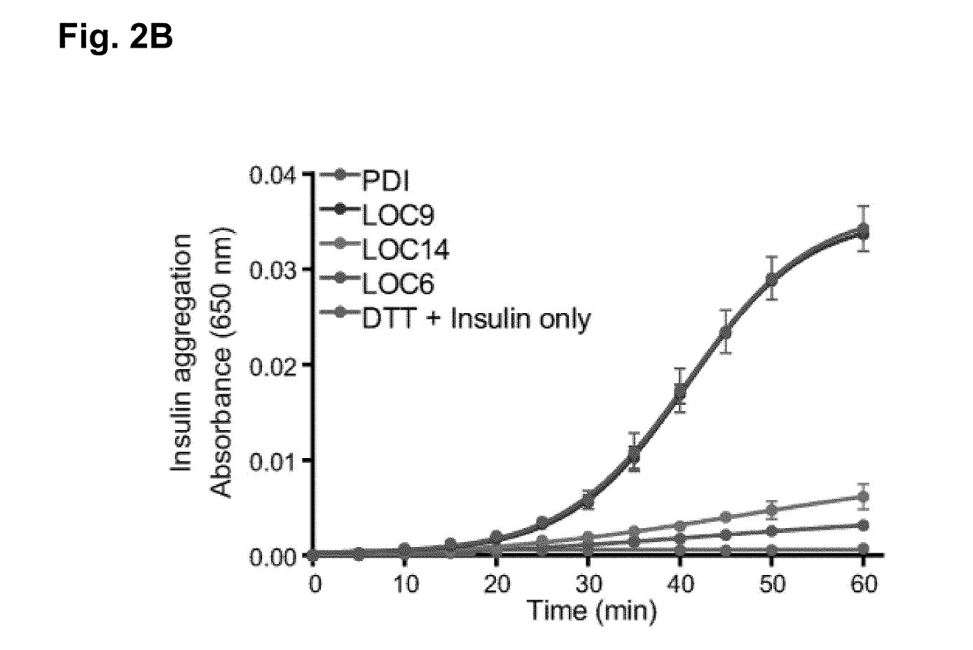

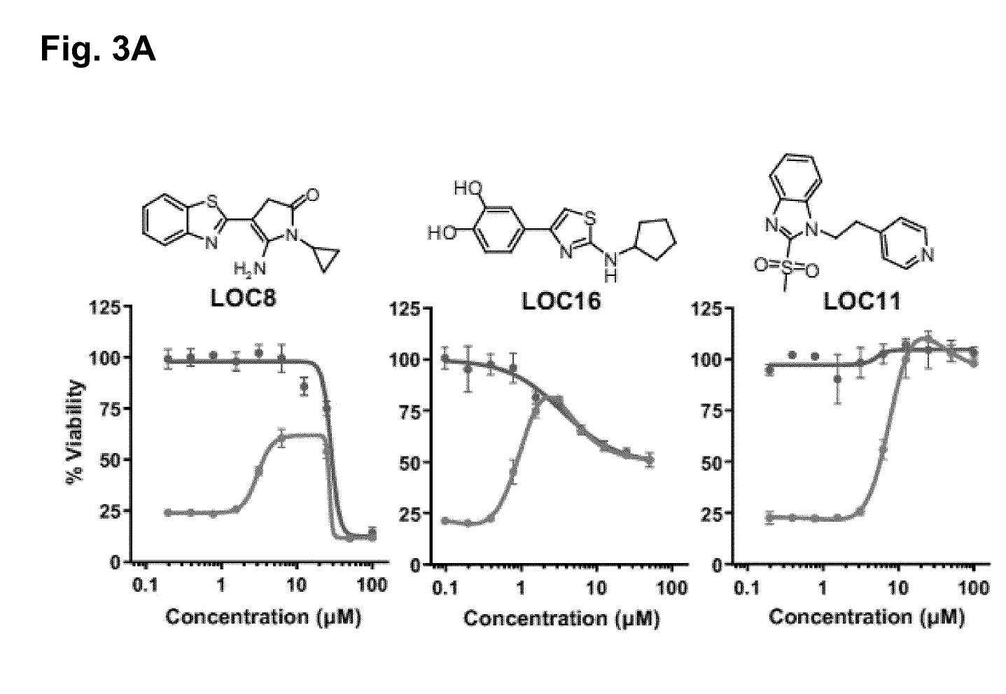

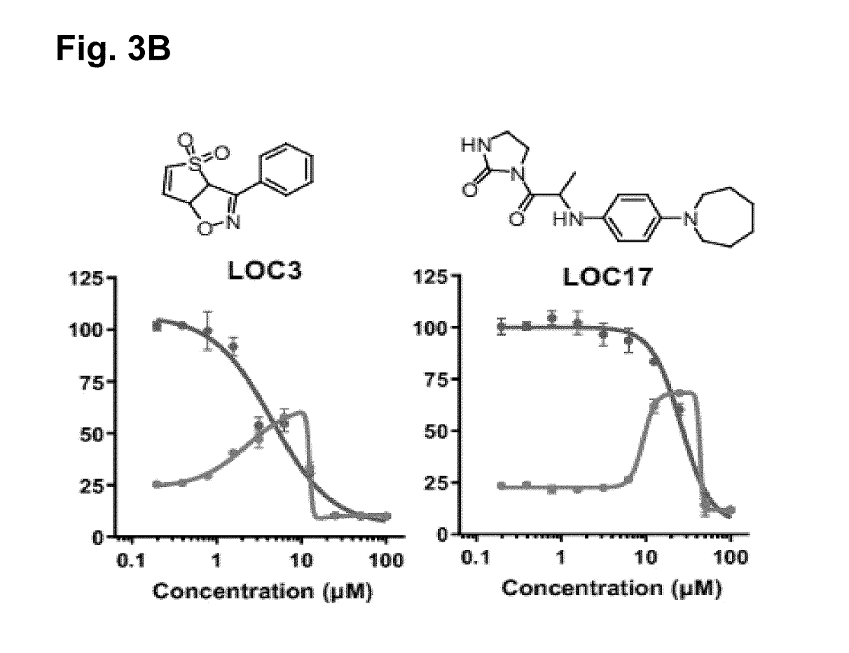

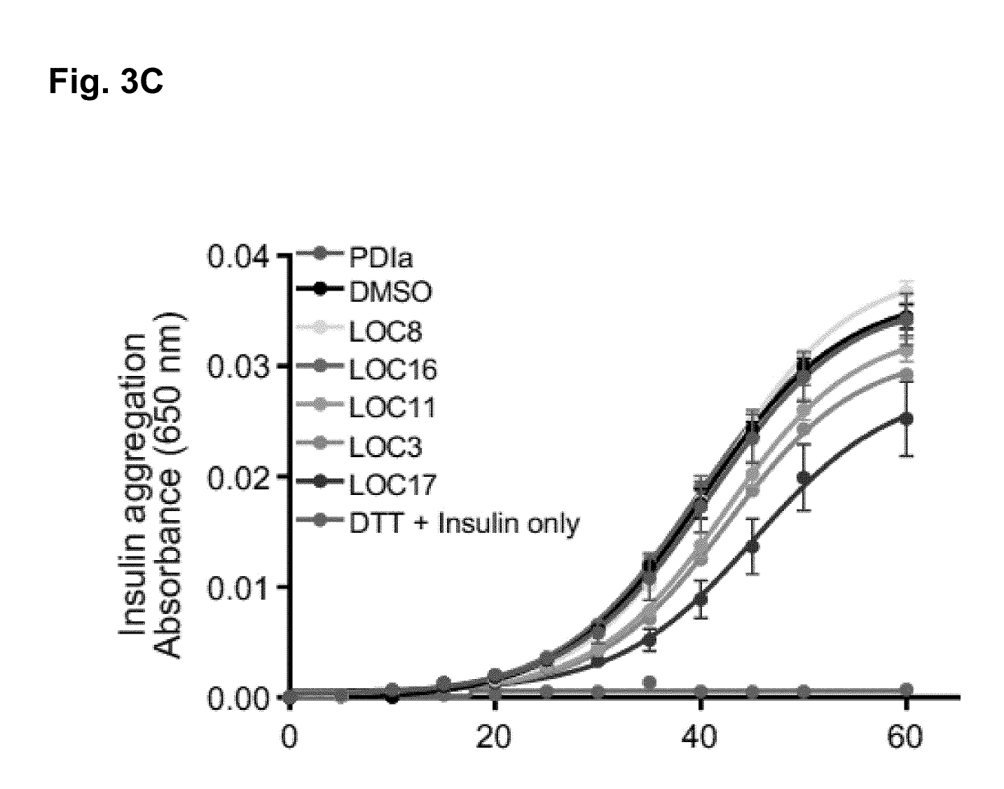

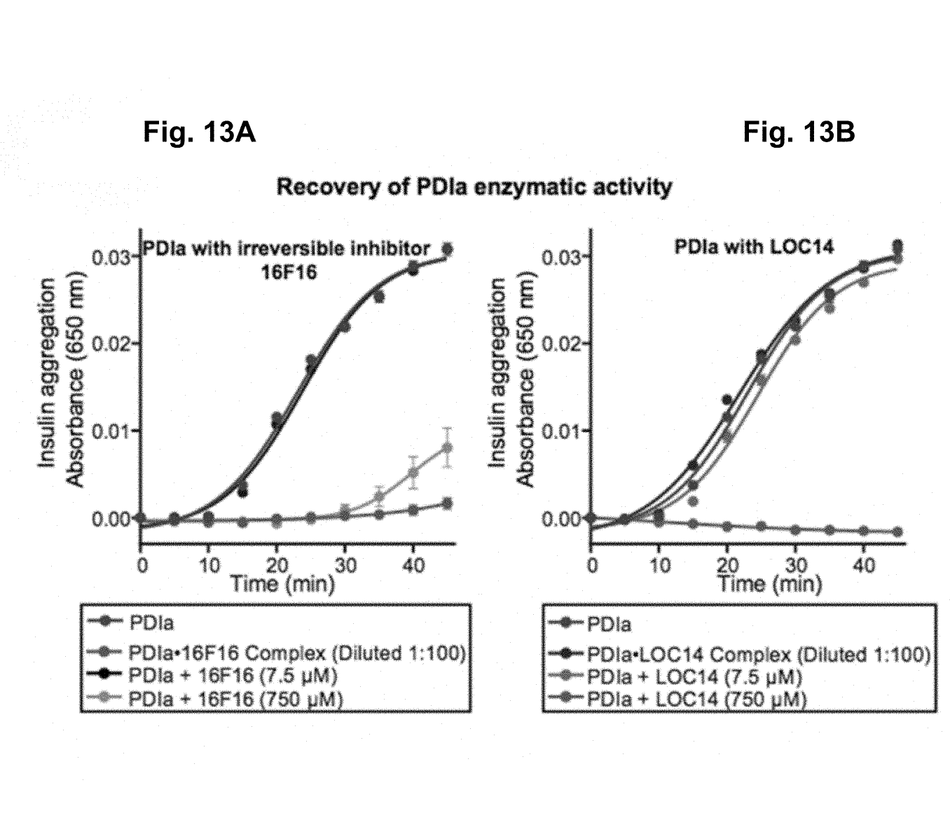

A cascade of two assays was used, involving both a phenotypic high-throughput screening (HTS) assay and an in vitro PDI reductase assay, to identify neuroprotective PDI-inhibiting compounds. (FIG. 13A-FIG. 13B) PC12 cells stably transfected with an inducible plasmid for mutant huntingtin protein (Aiken et al., 2004) (mHTTQ103) were used for the screen, because they previously showed reliance on PDI inhibition for survival from misfolded mHTT^{Q103}-induced cell death (Hoffstrom et al., 2010). Each compound in the LOC library was screened in triplicate at three different concentrations, 4 μg/ml, 1 μg/ml, and 0.25 μg/ml, resulting in nine data points per compound, in order to maximize the probability of identifying effective compounds. Alamar blue was used as a fluorescent readout for viability after 48 hours of compound treatment and mHTT^{Q103} induction. The overall Z′ factor for the screen was 0.78 with a signal-to-noise ratio at 165 and coefficient of variation of 5.8%, indicating a robust assay for hit identification (Zhang 1999). Out of 9,719 compounds, nine compounds rescued PC12 mHTT^{Q103} cells to at least 45% viability in the primary screen. All the candidate hit compounds were re-tested in a two-fold dilution series. The viability curves of the eight compounds that reproducibly exhibited >50% viability are shown in FIGS. 2A and 3A. Out of these, three compounds had EC₅₀ values in the nanomolar range; two compounds, LOC14 (EC₅₀=500 nM) and LOC9 (EC₅₀=600 nM) (FIG. 2A) were more potent than the previously identified irreversible neuroprotective PDI inhibitor 16F16 (EC₅₀=1000 nM) (Hoffstrom et al., 2010).

Because neuroprotection of PC12 mHTT^{Q103} cells can occur via additional pathways other than PDI modulation, e.g., caspase inhibition, the hits from the cell-based assay were screened for inhibition of PDI's reductase activity using insulin and the recombinant catalytic a domain of human PDI A1 (referred to as PDIa), which can perform the same catalytic oxidation and reduction reactions as full length PDI with one inactive domain (Darby & Creighton, 1995). In this insulin aggregation assay (Xu et al., 2012; Jasuja et al., 2012; Khan et al., 2011; Smith et al., 2004), PDIa reduced the two disulfide bonds between the α- and β-chains of insulin, causing the β-chain to aggregate and precipitate, resulting in an increase in absorbance at 650 nm. Out of eight hit compounds from the cell culture screen, two, LOC14 and LOC6, were able to almost completely inhibit PDIa enzymatic activity (FIG. 2B and FIG. 3C).

From these stages of screening, LOC14 emerged as the most potent small molecule that could both rescue PC12 mHTT^{Q103} cells and inhibit PDIa reductase activity; therefore LOC14 was selected as a lead compound for further analyses.

### Example 4

### LOC14 Binds with Nanomolar Affinity to PDI

To confirm the compound's identity, LOC14 was resynthesized (Materials and Methods and FIG. 1). The biochemical activity of the resynthesized LOC14 was identical to the commercially obtained compound.

The binding mode of LOC14 to PDIa was next investigated using isothermal titration calorimetry (ITC). ITC measures the heat released or absorbed during a biomolecular interaction. It is a direct analytical method for determining the binding and thermodynamic parameters, such as reaction stoichiometry (n), binding constants (Kₐ and K_{d}), enthalpy (ΔH), entropy (ΔS), and free energy (ΔG) of an interaction.

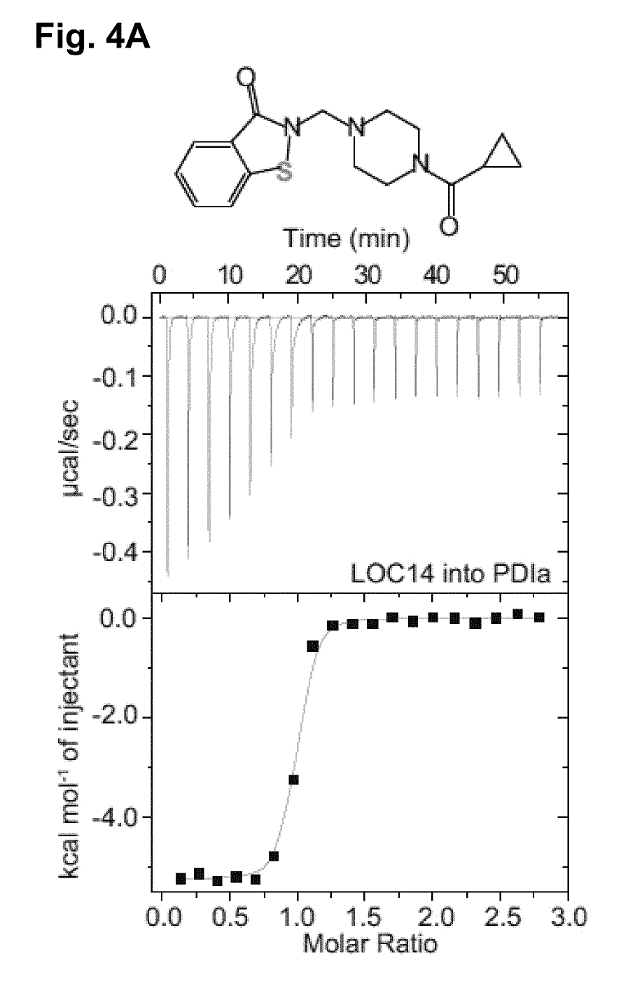

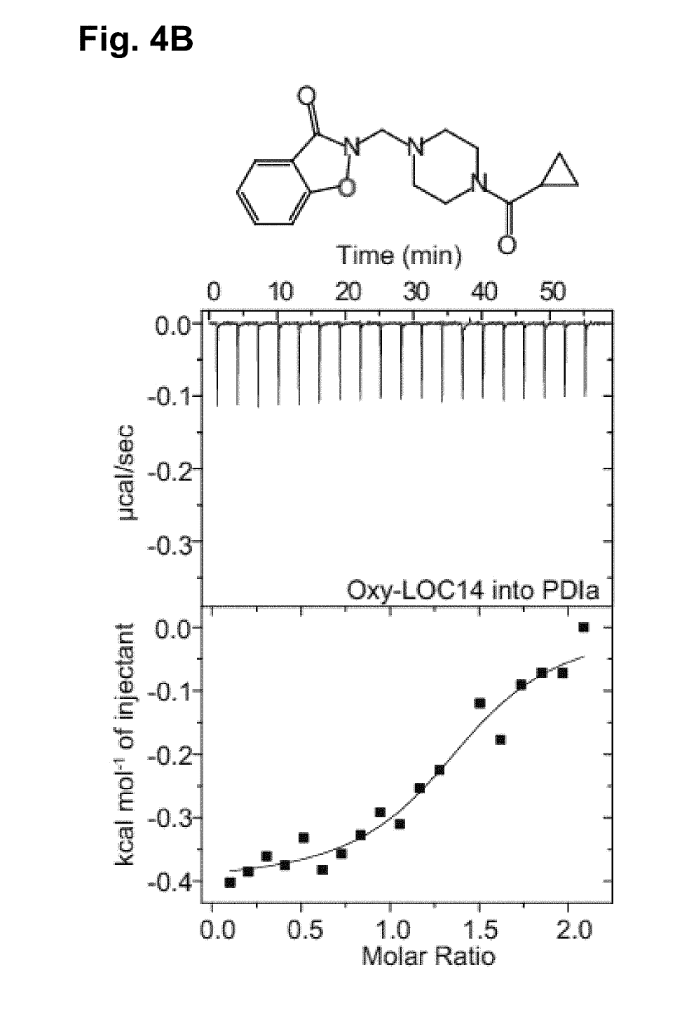

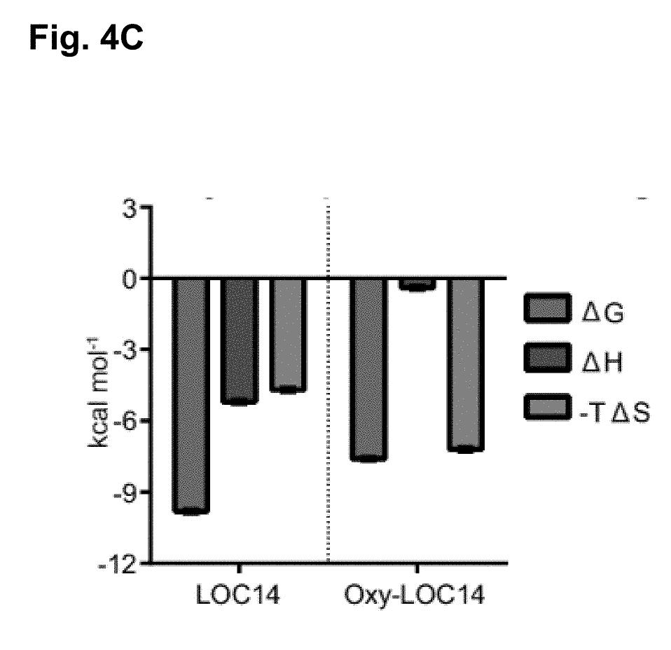

Calorimetric titration of LOC14 against PDIa showed an exothermic binding (FIG. 4A) with a dissociation constant (K_{d}) of 61.7±5.6 nM. The compound titration into buffer alone was subtracted from the raw binding data to account for the heat of dilution. The thermodynamic parameters plot (FIG. 4C, left panel) showed that the overall favorable affinity of LOC14 to PDIa was driven by both favorable (negative) enthalpic and entropic contributions. This finding indicated that likely both polar and hydrophobic interactions contribute to LOC14 binding affinity to PDIa.

To test if the sulfur atom on LOC14 is important for interaction with the protein, an isoxazolone analog of LOC14 was synthesized, termed Oxy-LOC14 (Materials and Methods) and its binding affinity to PDIa tested. Oxy-LOC14 had a 35-fold loss in binding affinity compared to LOC14 and a K_{d} of 2,433±764 nM by ITC (FIG. 4B). The thermodynamic parameters plot showed that Oxy-LOC14 had almost complete loss of its enthalpic binding component (FIG. 4C, right panel). This difference in the thermodynamic signatures due to a single atom, sulfur to oxygen, substitution indicated that the sulfur atom on LOC14 can form favorable interactions with the protein.

### Example 5

### LOC14 is a Reversible Modulator of PDI

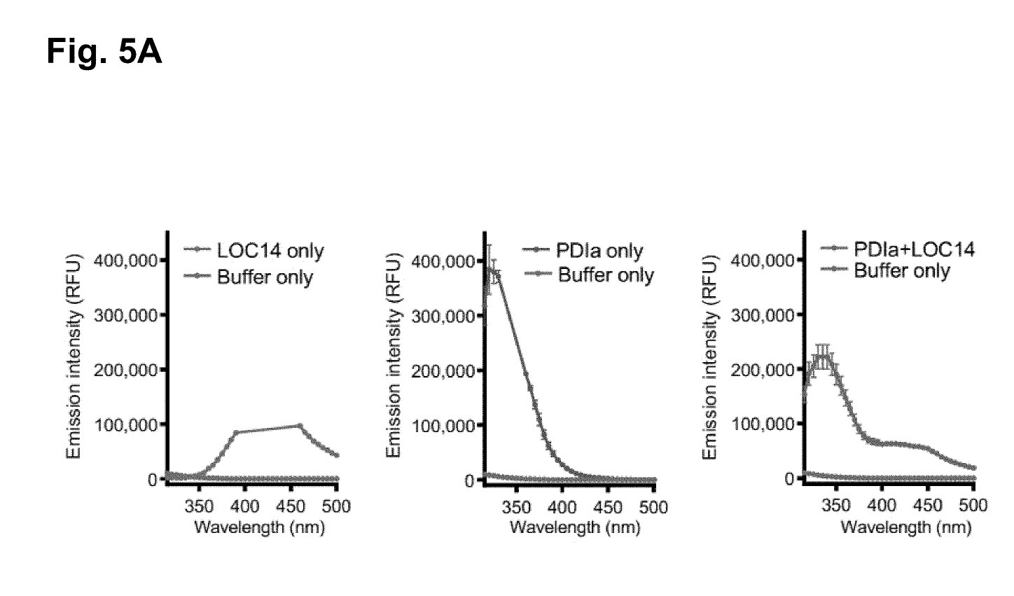

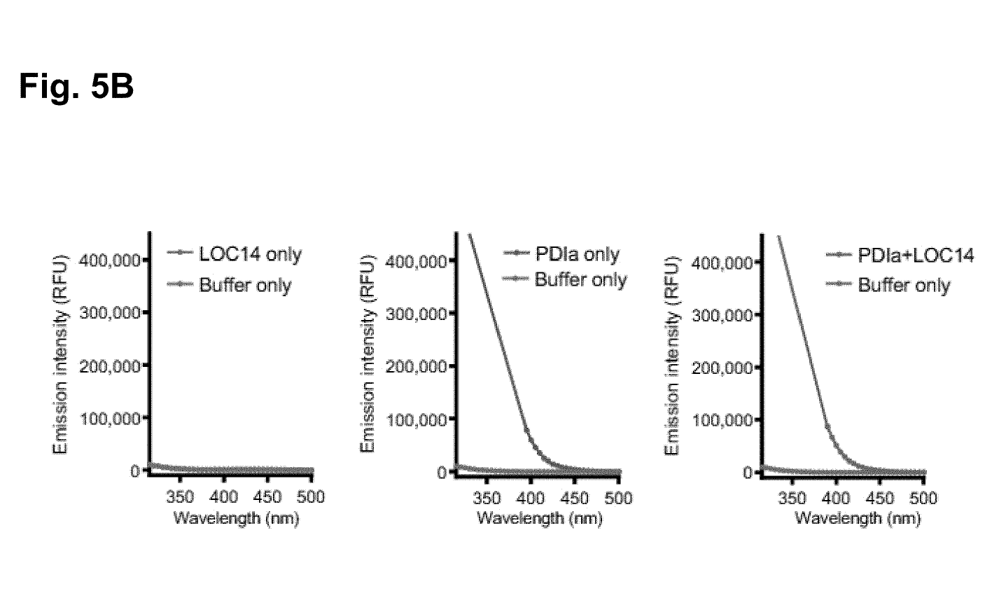

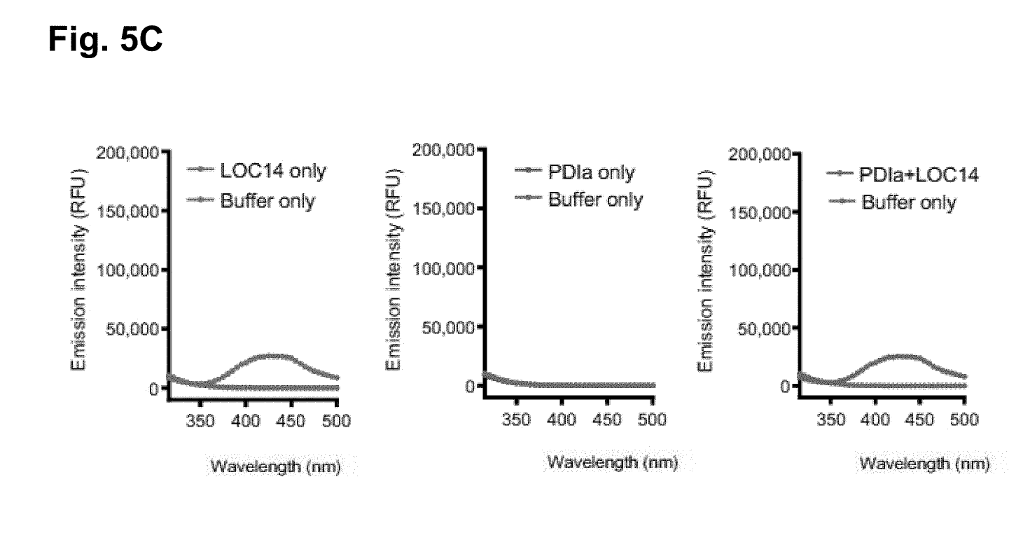

The importance of the sulfur atom on LOC14 suggested that it might be binding covalently to the active site of PDIa, which contained two cysteines with reactive thiol groups. To investigate the mode of binding further and to determine whether the binding was reversible or irreversible, the fluorescence of the LOC14-PDIa complex was analyzed before and after buffer dialysis. LOC14 had a distinct emission spectrum when excited at 280 nm that is different than the emission of PDIa alone, or from the LOC14-PDIa complex (FIG. 5A). If LOC14 binds irreversibly to the protein, then the same fluorescence spectrum of the LOC14-PDIa complex would be seen before and after dialysis in the dialysis chamber and none in the buffer compartment, assuming the fluorescence of LOC14 was not dramatically altered upon binding. LOC14 and PDIa were incubated together overnight (to allow for the maximum binding to occur in the case that LOC14 was a time-dependent irreversible binder) and dialyzed the next day with buffer four times using an Amicon Ultra 10 kDa cut-off size exclusion filtration device. As a control, samples that contained only PDIa or LOC14 were also used. The emission spectrum was recorded of samples from the flow-through and dialysis chamber. In the control samples, the fluorescence of LOC14 alone was observed only in the flow-through fraction while the fluorescence of PDIa alone was only observed in the dialysis compartment (FIGS. 5B and 5C) as was expected. Furthermore, their respective emission spectra were identical before and after the buffer exchange. The PDIa treated LOC14 sample however, showed a different fluorescence profile before and after dialysis (FIGS. 5B and 5C). The emission spectrum in the dialysis chamber resembled PDIa alone fluorescence, and the flow-through fraction resembled the fluorescence profile of LOC14 only. This illustrated that LOC14 binding to PDIa was reversible.

To investigate the mode of binding further and to determine whether the binding was reversible or irreversible, the recovery of enzymatic activity of PDIa after dilution of pre-formed concentrated PDIa-LOC14 complexes was analyzed. LOC14 (750 μM) was incubated with a concentrated solution of PDIa (500 μM). The mixture was then diluted 100-fold and PDI's enzymatic activity was measured in the insulin reduction assay. As a control, the same procedure was also performed on the sample containing an irreversible inhibitor, 16F16 (16F16 at 750 μM and PDIa at 500 μM). Without dilution, concentrated 16F16 (750 μM) (FIG. 13A, green) or concentrated LOC14 (750 μM) (FIG. 13B, gray) were both able to inhibit PDI's reduction of insulin. However, after dilution, the samples containing LOC14-PDIa diluted complexes showed complete recovery of PDI's enzymatic activity that lead to insulin reduction and precipitation (FIG. 13B, blue). PDI's enzymatic activity was still inhibited, after dilution, in samples that contained 16F16-PDIa diluted complexes (FIG. 13A, purple). This illustrated that unlike 16F16, LOC14 binding to PDIa is reversible.

### Example 6

### LOC14 Binds Adjacent to the Active Site and Oxidizes PDI

To identify residues on PDIa involved with the LOC14 interaction, ¹H-¹⁵N heteronuclear single quantum correlation (HSQC) binding studies were performed on the uniformly ¹⁵N-labeled PDIa with and without LOC14. ¹H-¹⁵N HSQC spectra displays directly bonded N—H resonance peaks from each amino acid in a protein. Upon ligand binding, the perturbations in the local environment induced a change in the chemical shift of the resonance peaks. Only the residues that were either involved in the binding site or in the conformational change of the protein upon compound binding were perturbed (Williamson, 2013). Knowing the resonance assignments is beneficial when mapping the binding site from chemical shift perturbation data.

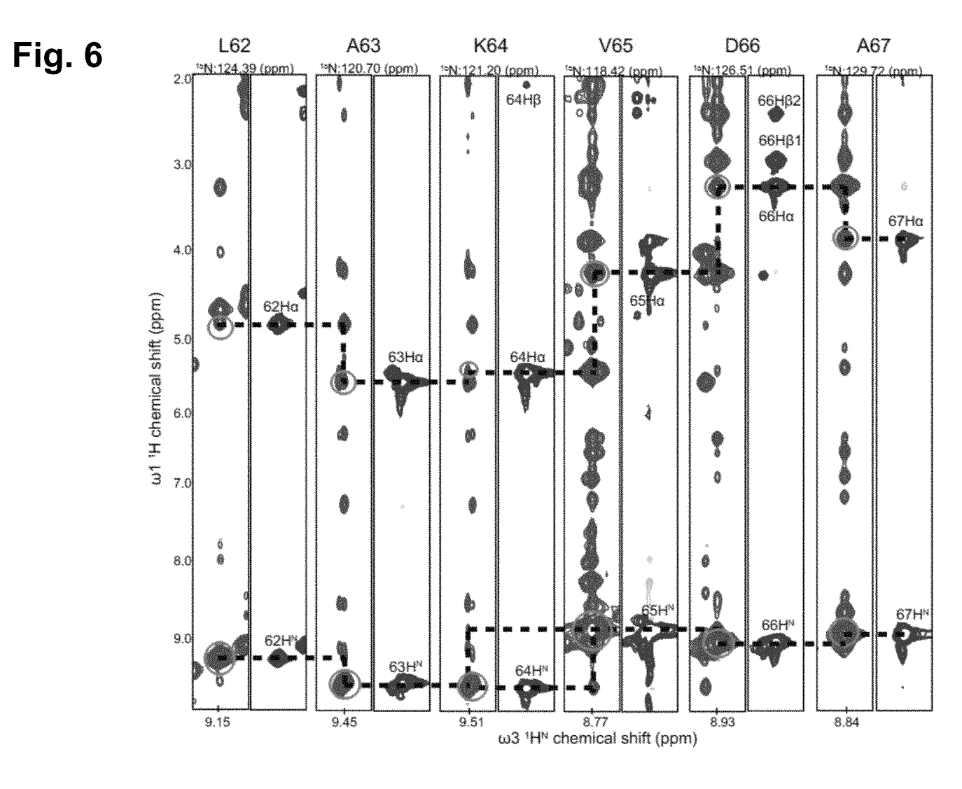

The resonance assignments of oxidized PDIa have been previously determined (Kemmink et al., 1995); however, the resonances for the reduced form of the protein were not known. By matching the conditions reported by Kemmink et al., the assignments of unaltered residues in the oxidized protein were able to be efficiently transferred to the peaks of ¹H-¹⁵N HSQC spectrum of reduced PDI protein. To validate the assignments ¹⁵N-TOCSY-HSQC and ¹⁵N-NOESY-HSQC were performed (FIG. 6). This 3D-NMR data was then used to identify the individual spin systems and sequentially assign the reduced PDIa protein.

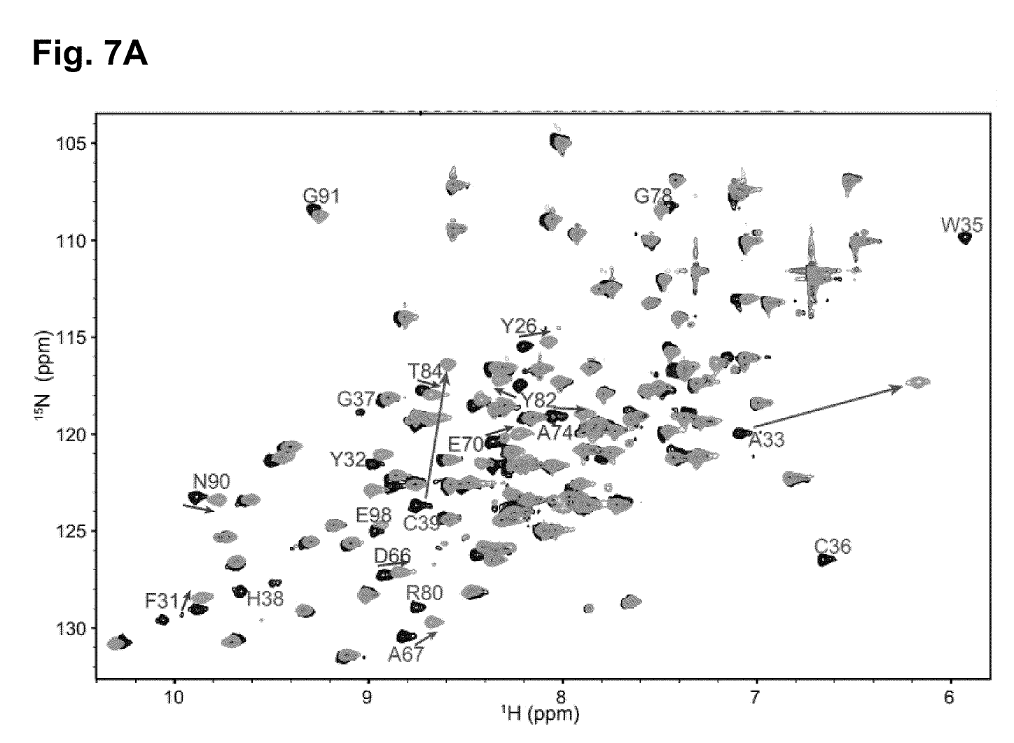

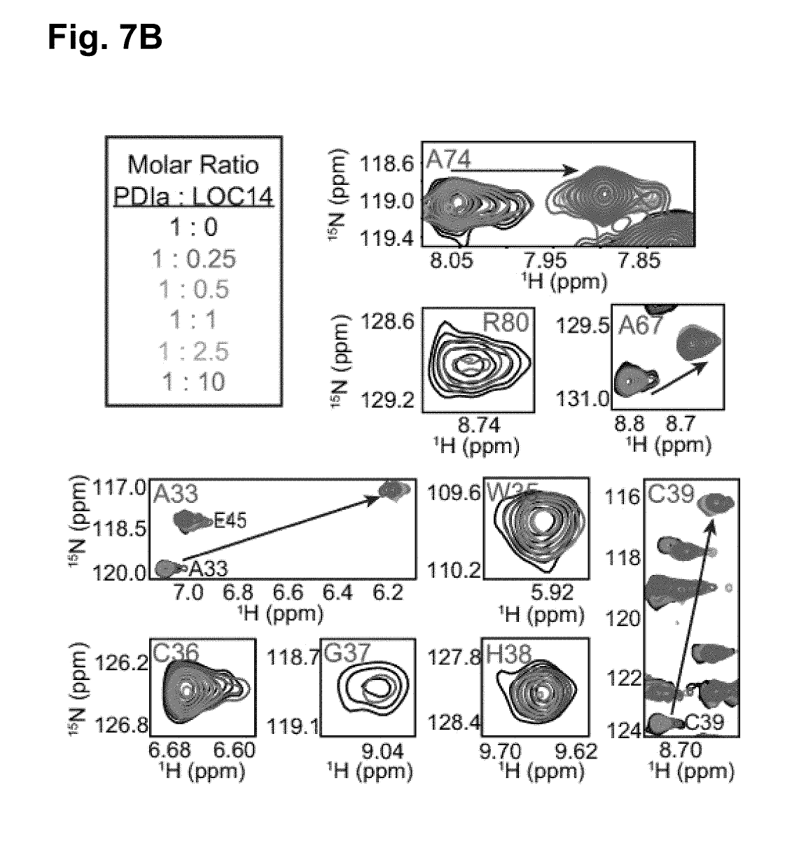

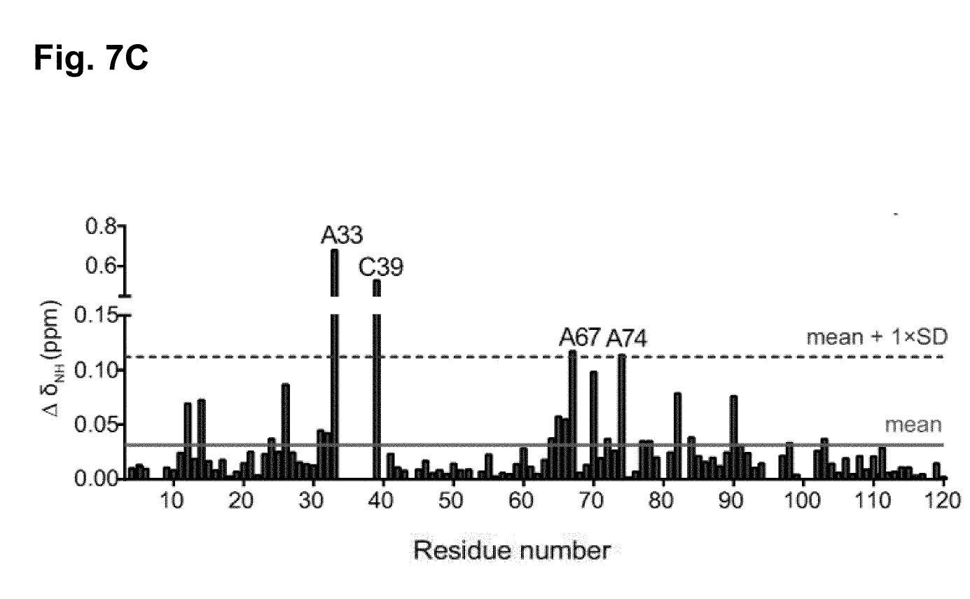

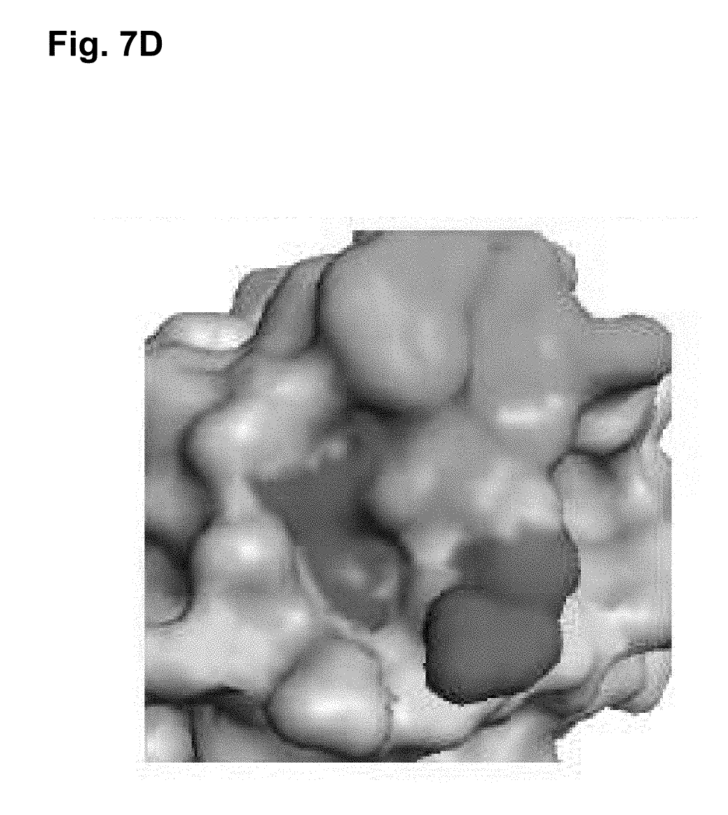

The resulting HSQC spectrum with one-to-one molar equivalence ratio (PDIa to LOC14) upon LOC14 binding is shown in FIG. 7A. Titrating LOC14 beyond one to one (protein:compound) molar ratio resulted in no additional shift changes (FIG. 7B), indicating that by one molar equivalent, the protein was fully saturated with ligand. This result is consistent with the calculated K_{d} data from the ITC experiments that determined nanomolar affinity of LOC14 for PDIa. The lowest concentration of reduced PDIa that could be used for optimal sensitivity in HSQC experiments was 50 μM and, as this is above the nanomolar K_{d}, one-to-one stoichiometric binding was expected.

Furthermore, it was observed that the majority of the peaks that were shifting were undergoing a slow exchange upon compound titration. This was evident upon an observable decrease in peak intensity and then either an abrupt appearance in a new location or a complete disappearance of the peaks all together (FIG. 7B). This slow exchange on the NMR timescale was indicative of tight binding and conformational change in the protein.

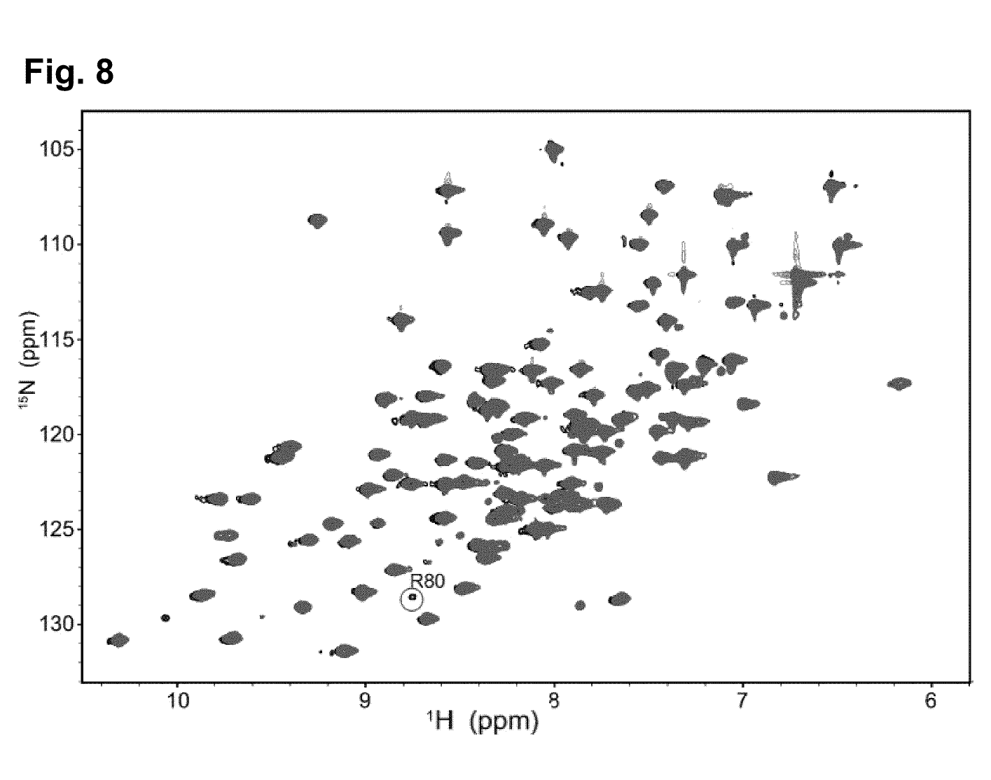

Closer examination of HSQC spectrum of reduced PDIa liganded to LOC14 revealed that upon compound binding, PDIa adopts an oxidized conformation. This was evident by the perfect overlay between the HSQC spectrum of oxidized protein alone and the HSQC spectrum of reduced PDIa liganded with LOC14 (FIG. 8). One major difference between these two, however, was seen in residue R80. The resonance peak for R80 was present in the oxidized PDIa HSQC spectrum, but disappeared when the reduced PDIa is treated with LOC14. This was indicative of protein-ligand interaction. All other residues that disappeared upon compound treatment such as W35, C36, G37, H38, were absent in the oxidized protein HSQC spectrum.

Mapping the residues with the most significant chemical shift change (FIG. 7C) and residues that disappeared upon compound titrations to the structure of PDIa illuminated a small area between the active site and R80 (FIG. 7D). The few residues not localized to this pocket, e.g., L12, K14, Y26 and N90, were involved in the conformational change of the protein upon ligand binding.

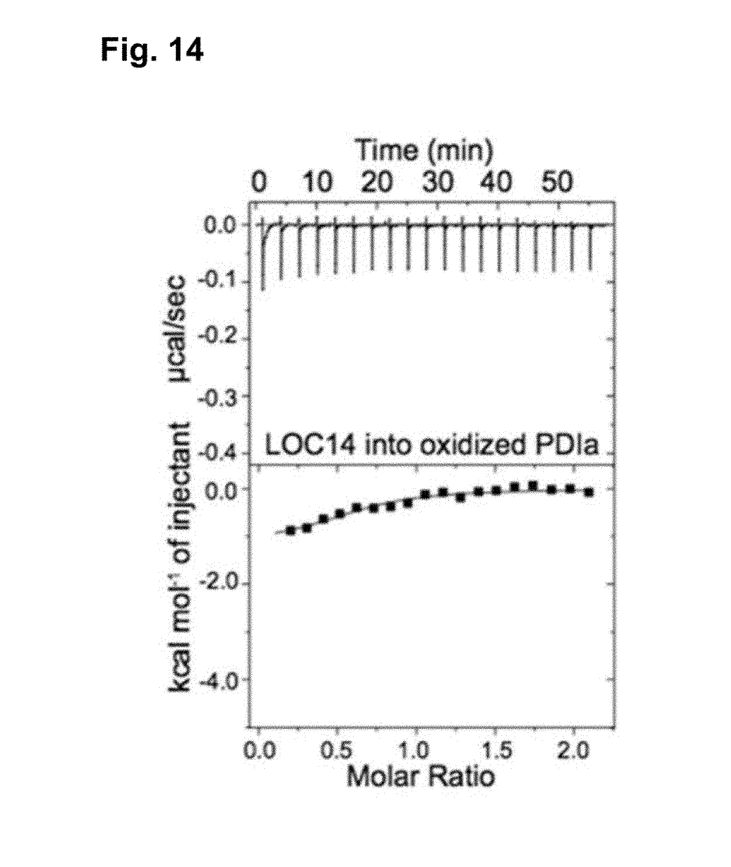

Calorimetric titration of LOC14 against oxidized PDIa showed a loss in binding affinity (compared to LOC14 titration into reduced PDIa) (FIG. 14). These data indicate that the presence of free cysteine thiols on PDIa are important for the tight interaction with LOC14. Additionally, this reinforces that the observed HSQC shifts that reflect oxidative conformational change in PDIa are due to LOC14 binding.

### Example 7

### LOC14 Binds to a Different Site than 16F16

Based on the mapped binding site from the NMR studies and the fact that sulfur atom was essential to retain tight binding of LOC14 to the protein, it was next examined whether LOC14 was binding to the two cysteines in the protein, C36 and C39, both in the active site.

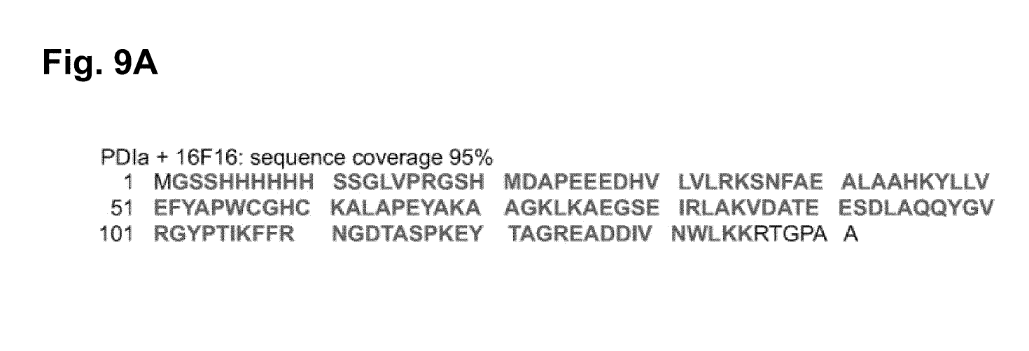

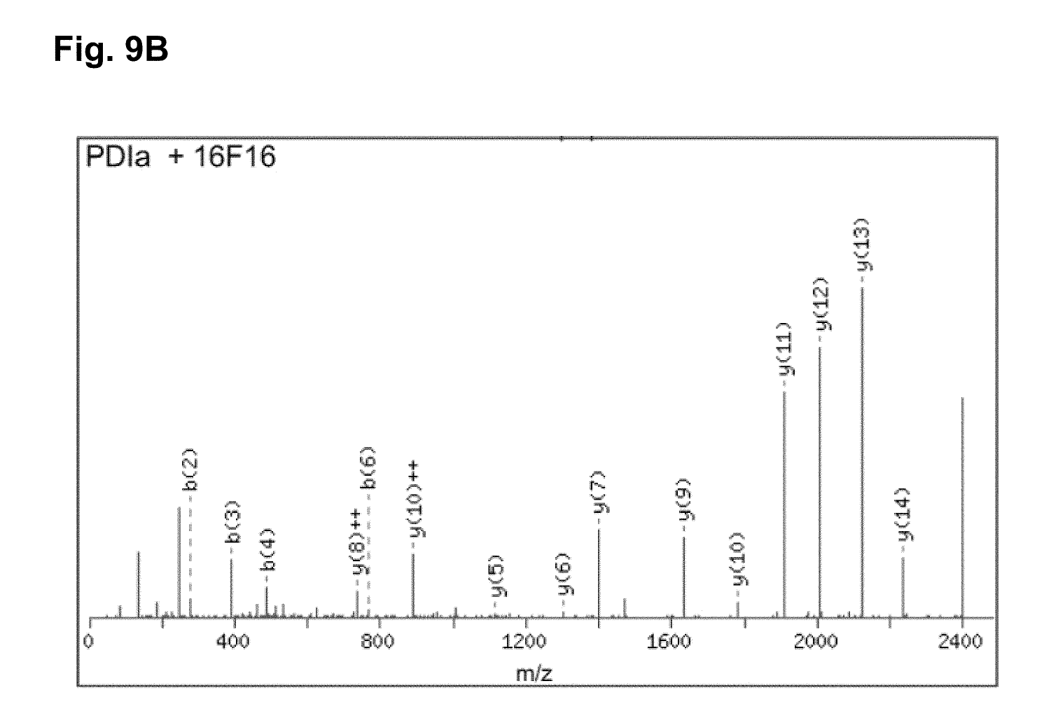

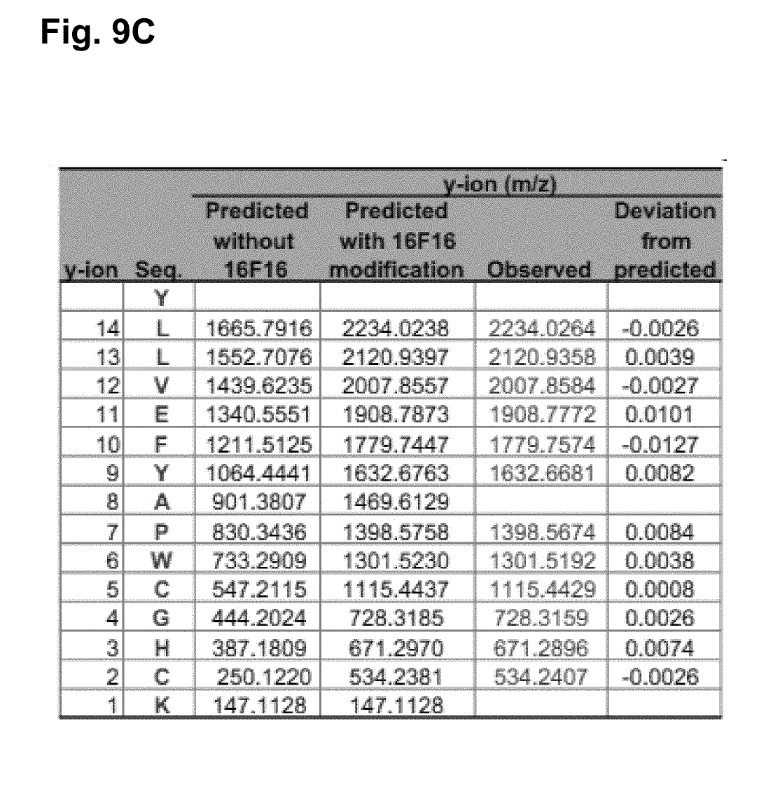

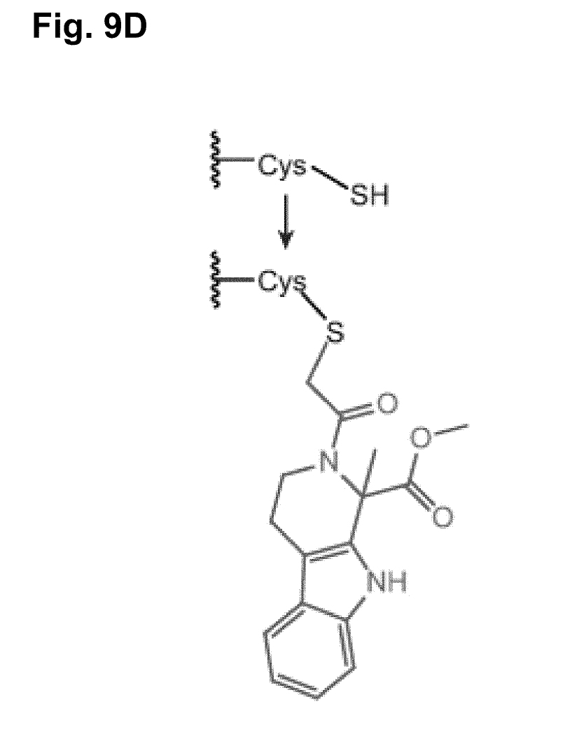

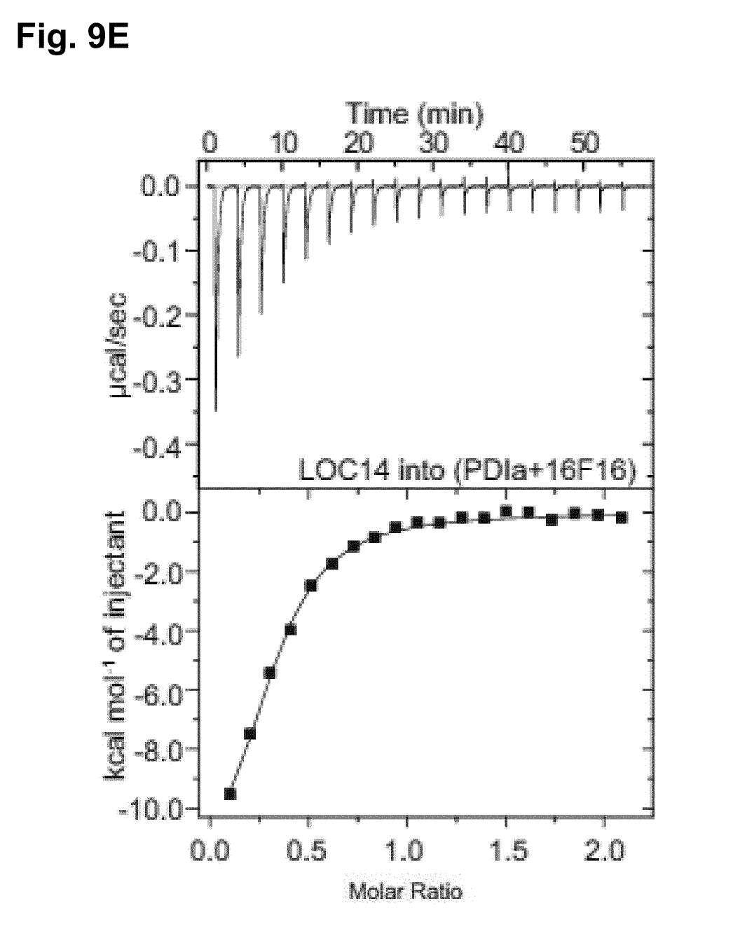

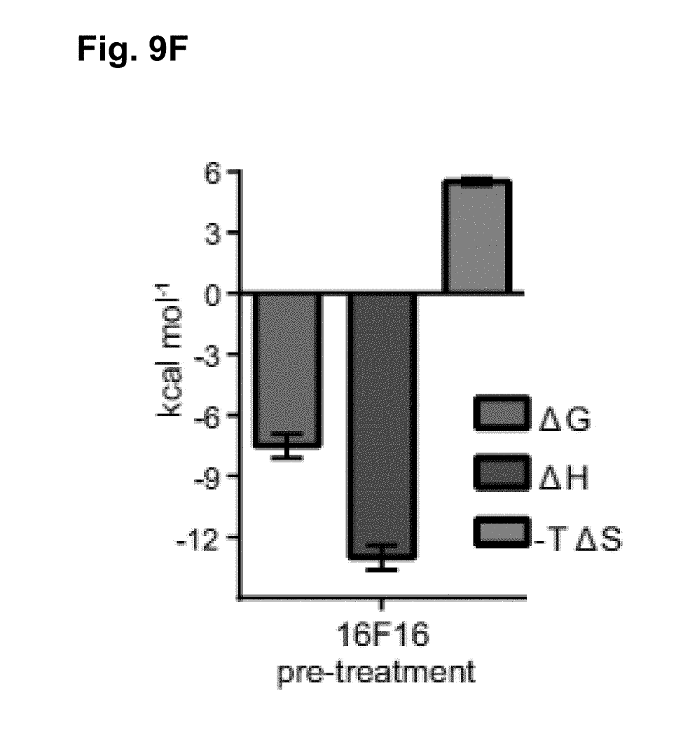

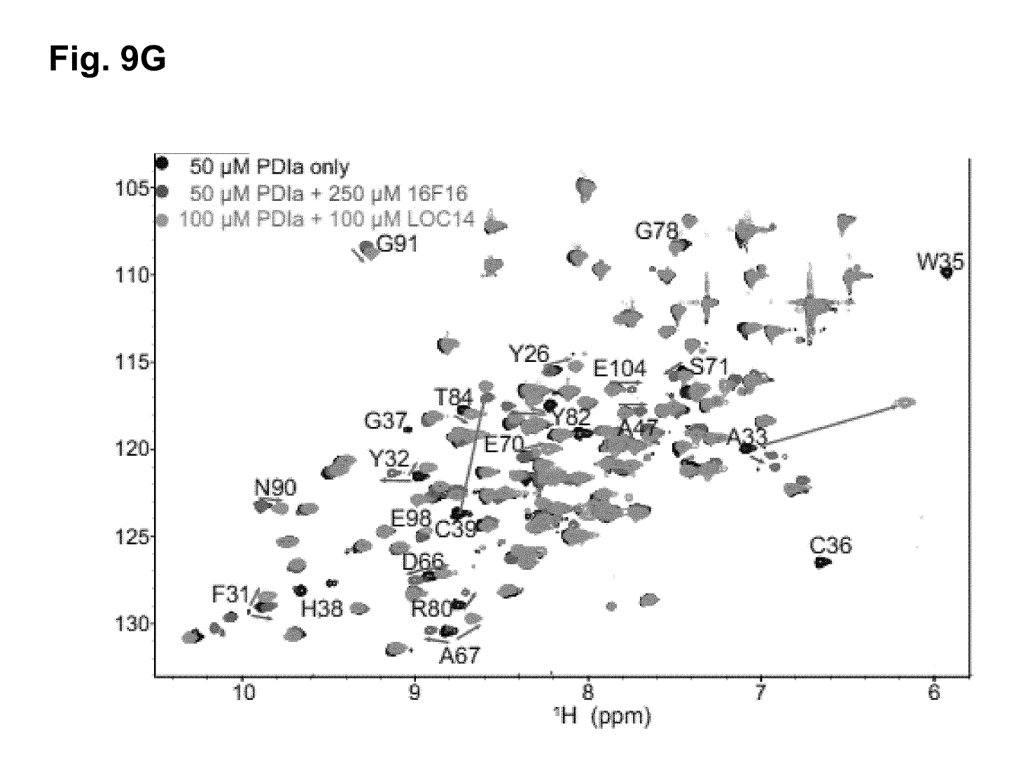

Previously, 16F16 was reported to function as an irreversible inhibitor of PDI A1 and PDI A3 proteins (Hoffstrom et al., 2010). The compound 16F16 contains a chloroacetyl group that covalently modifies free cysteine thiols. To confirm the likely covalent nature of the interaction and identify the cysteines on PDIa modified by 16F16, LC-MS/MS fragmentation was performed. Compound 16F16 selectively bound to the only cysteines in the PDIa protein and it was able to covalently modify both C36 and C39 (FIGS. 9A-9D). To test if LOC14 bound to the same active site cysteines as 16F16, PDIa was pre-incubated with 16F16 overnight and then analyzed by ITC upon LOC14 titrations. After pre-treatment, the two thiols in the active site were irreversibly bound to 16F16 and not available for LOC14 interaction. ITC data showed that the binding affinity was reduced with 16F16 pre-treatment, but not completely obliterated (FIG. 9E).

Additionally, the thermodynamic parameters plot (FIG. 9F) showed a different mode of binding than when the protein was treated with LOC14 alone (FIG. 4C). Even though the overall ΔG of binding was favorable (negative), there was a large entropic penalty (positive ΔS) when LOC14 bound, most likely due to the conformational change in the protein. This was also supported by NMR ¹H-¹⁵N HSQC data (FIG. 9G). PDIa treated with 16F16 displayed a different protein conformation, seen by the different chemical shift changes. Not only were there different residues involved with 16F16 binding, but even if the same residues were affected as when LOC14 binds, upon 16F16 treatment they had a different shift direction. Thus, it would make sense that the protein adopted one conformation when 16F16 was bound to the active site cysteines, most likely one that minimized the steric clash of having such a bulky group. Then, upon LOC14 binding, the protein was forced into another conformation, one that resembled its oxidation state, but paying the cost of unfavorable entropy.

The different mode of binding to PDIa between these small-molecule modulators is also supported by NMR ¹H-¹⁵N HSQC data (FIG. 9G). PDIa treated with 16F16 displays a different protein conformation, seen by the different chemical shift changes, than when LOC14 is bound to PDIa.

### Example 8

### LOC14 can Protect Medium Spiny Neurons from Neurotoxicity Induced by Mutant Huntingtin Protein

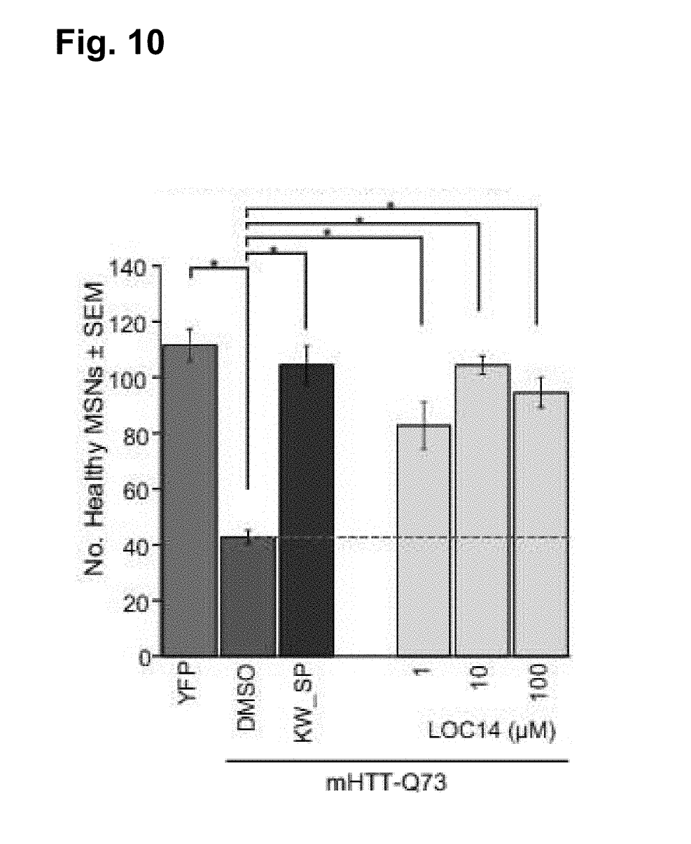

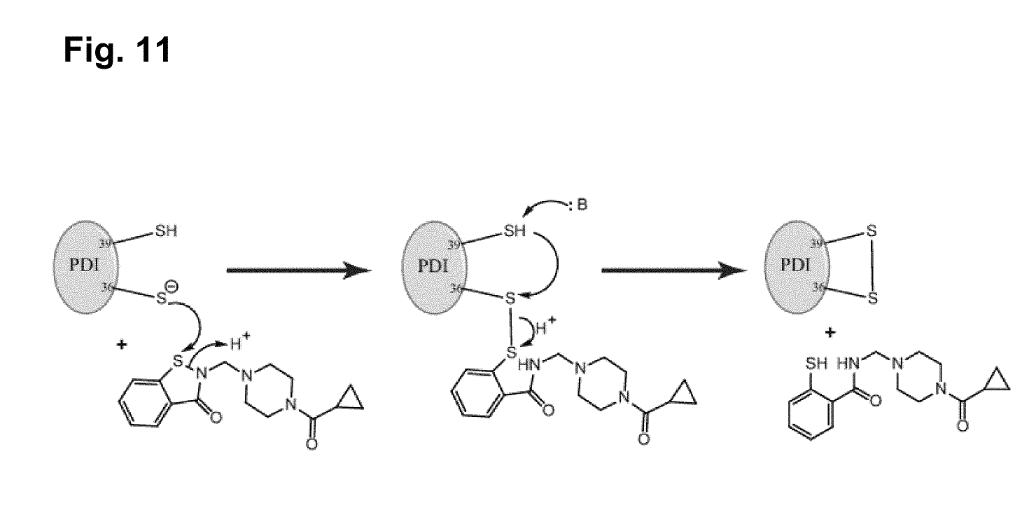

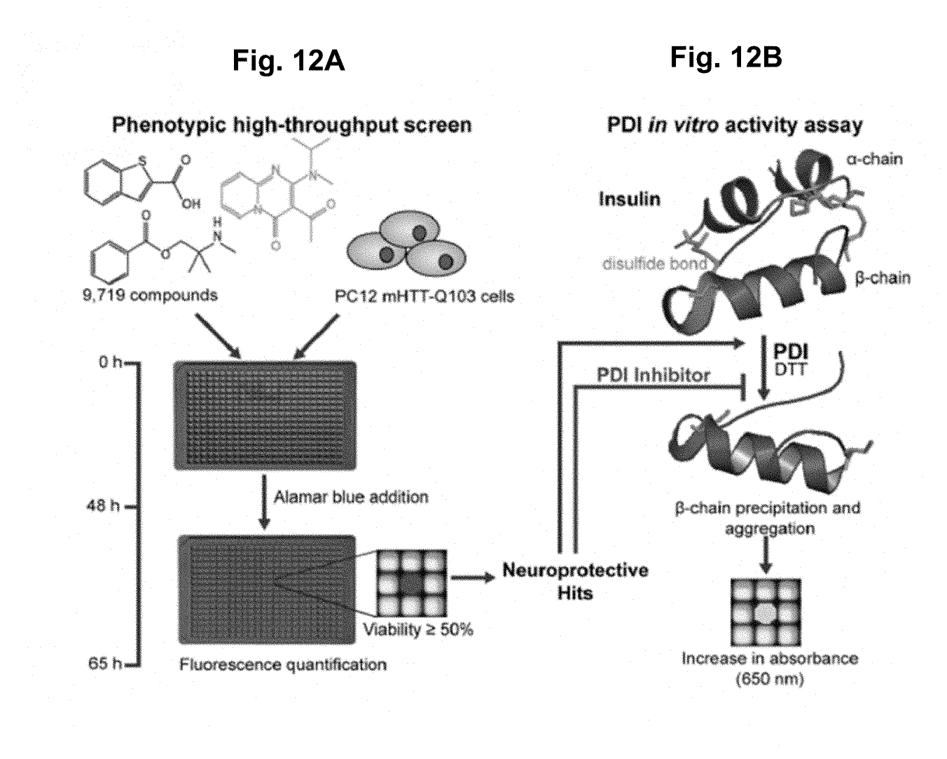

Having elucidated aspects of the biophysical mechanism of action of LOC14 binding to PDI, the next exploration concerned whether LOC14 would be a good candidate for in vivo studies. The activity of LOC14 in an organotypic postnatal brain slice model was examined for HD focusing on the medium spiny neurons (MSN) of the striatum (Reinhart et al., 2011). MSNs are the first population of neurons to degenerate in patients with HD and are the most vulnerable to the toxicity associated with mutant huntingtin dysfunction. Rat corticostriatal brain slice explants were co-transfected with YFP and the first exon of mutant HTT gene (mHTT-Q73) to induce neurodegeneration, and then treated with LOC14. In the absence of LOC14, very few healthy MSNs remained, as assessed by the lack of normal sized and shaped cell bodies, absence of long primary dendrites, and lack of continuous expression of YFP throughout the cell (FIG. 10). Compound LOC14 rescued MSNs in a concentration-dependent manner, even at low micromolar concentrations (FIG. 10). This indicated that LOC14 oxidation of PDI is neuroprotective in both cell culture and brain tissues.

### Example 9

### LOC14 is Metabolically Stable Compound for In Vivo Studies

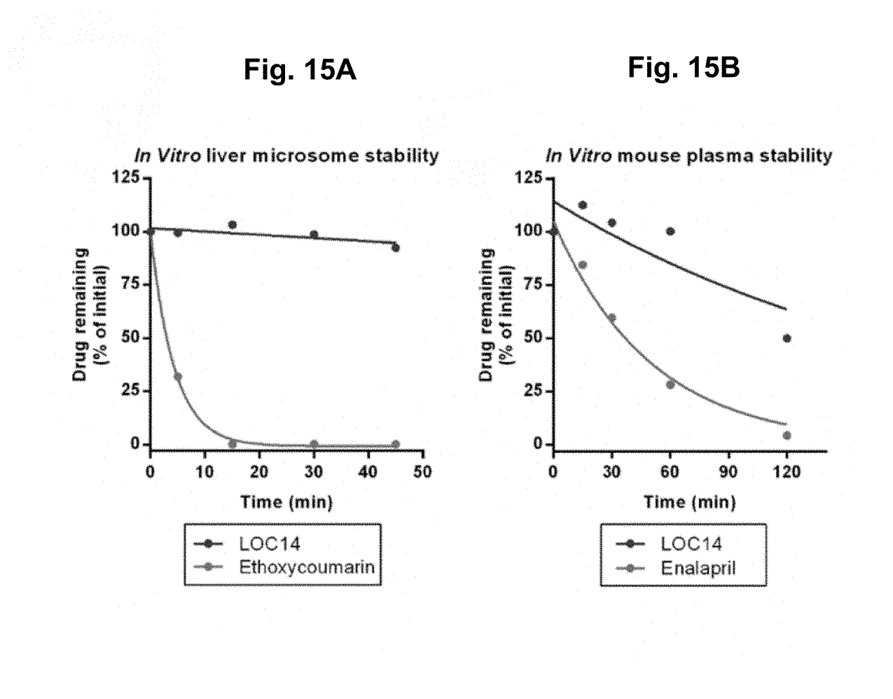

Next, metabolic in vitro stability studies were performed with LOC14 to determine its suitability for in vivo studies. LOC14 showed high stability in mouse liver microsomes, had a low intrinsic clearance value of less than 0.5 ml/min/g, and a half-life of more than 90 minutes (Table 1, FIG. 15A). This indicated that LOC14 was not metabolically reactive with liver enzymes such as cytochrome P450s and may have a suitably long half-life in vivo. LOC14 was also relatively stable in mouse plasma with a half-life of 2.4 hours (Table 2, FIG. 15B). Furthermore, low binding was observed between LOC14 and the plasma proteins (Table 3), indicating that in vivo, the bulk of LOC14 was free to be distributed to tissues to exert pharmacological effects.

TABLE 1
Intrinsic clearance of LOC14 in mouse liver microsomes.

| | | | Intrinsic Clearance |
|---|---|---|---|
| | Elimination rate | Half-life | (CLᵢₙₜ), mL/min/g |
| Compound | constant (k) | (t_{1/2}), min | liver |
| LOC14 | 0.0016 | 438.7 | 0.17 |
| 7-Ethoxycoumarin | 0.2341 | 3.0 | 24.58 |

Compound concentration was 0.5 μM. 7-Ethoxycoumarin, a substrate of cytochrome P450 enzymes, was used as a control.

TABLE 2
Stability of LOC14 in mouse plasma.

| | | | % Remaining |
|---|---|---|---|
| | Elimination rate | Half-life | after 2-hour |
| Compound | constant (k) | (t_{1/2}), hours | incubation |
| LOC14 | 0.0049 | 2.4 | 49.9 |
| Enalapril | 0.0201 | 0.6 | 4.3 |

Compound concentration was 1.0 μM. Enalapril, which undergoes degradation in plasma, was used as a control compound.

TABLE 3
LOC14 plasma protein binding.

| Compound | % Bound | % Recovery |
|---|---|---|
| LOC14 | −18.76 ± 4.09 | 113.81 ± 0.76 |
| Warfarin | 94.21 ± 0.10 | 109.50 ± 3.54 |

Compound concentration was 2000 ng/mL. Warfarin, an anticoagulant, was used as a control compound. Data are shown as mean±SD (n=3).

### Example 10

### LOC14 can Protect Cortical Neurons from Neurodegeneration Induced by Tau

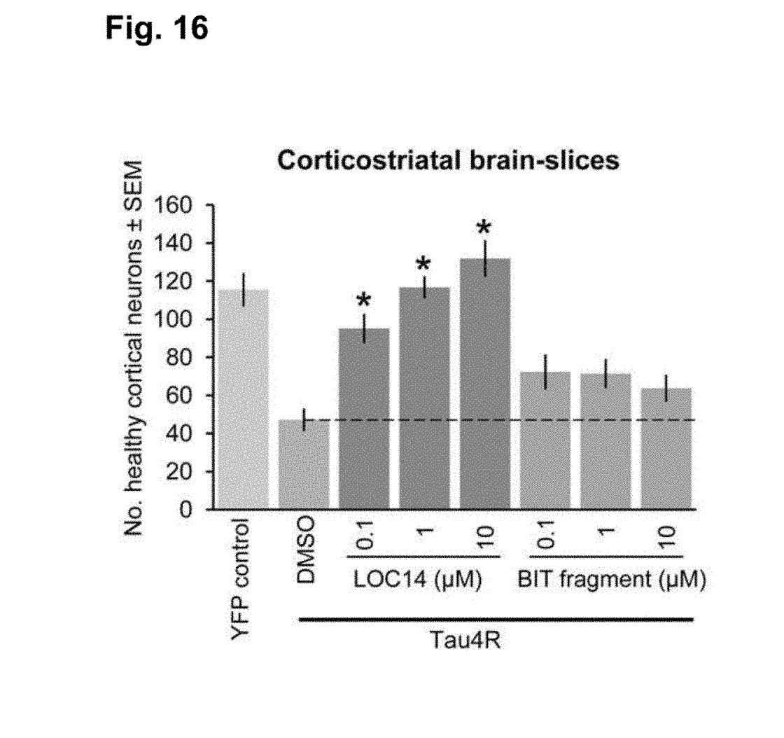

Next, whether LOC14 could be neuroprotective in additional models of neurodegenerative diseases was explored. It was found in fact that LOC14 is strongly and reproducibly neuroprotective in a tau-mediated neurodegeneration assay (FIG. 16). In this rat corticostriatal brain slice assay, cortical neuron degeneration is induced by biolistic transfection with tau, in this case a tau isoform with 4 tubulin-binding repeats (“tau4R”) which is implicated in frontotemporal dementias and more broadly in Alzheimer's disease. This indicates that LOC14 oxidation of PDI is neuroprotective in multiple neuronal disease models with misfolded proteins.

### Example 11

### In Vivo Pharmacokinetic Study with LOC14

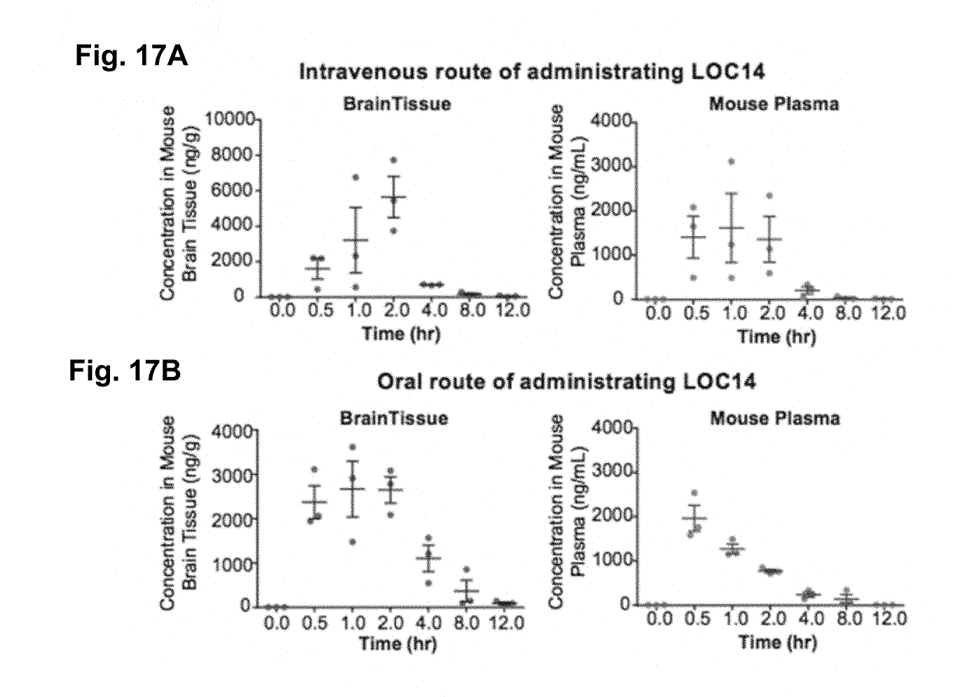

Showing promising in vitro metabolic properties, LOC14 was tested in a single-dose pharmacokinetic (PK) study. This was a pilot study to evaluate the ability of LOC14 to traverse the blood-brain barrier (BBB). In this study, LOC14 was administered via two routes, intravenously (FIG. 17A) or orally (FIG. 17B), at a single-dose of 20 mg/kg to wild-type C57BL/6j mice. Data from the PK study showed that LOC14 was well tolerated at high dose of 20 mg/kg, penetrated the BBB and accumulated at reasonable concentrations in the brain regardless of the administration route (FIGS. 17A and 17B)

### Example 12

### Design and Evaluation of LOC14 Analogs

Next, LOC14 analogs were designed and synthesized as described in the synthetic schemes. The analogs were evaluated for their binding to PDIa using isothermal titration calorimetry (ITC). The Kd values are given in Table 4. Next, the analogs were tested for their ability to rescue PC12 cells from mHTT^{Q103} induced cell death in a dose-dependent manner.

TABLE 4
Analysis of LOC14 Analogs.

| Our ID | Name | Structure | MW | Solubility | ITC |
|---|---|---|---|---|---|
| LOC14 | MMG509 MMG524 MMG821 MMG828 | 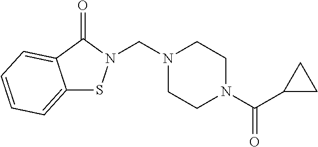 [SMILES: O=C(C1CC1)N1CCN(Cn2sc3ccccc3c2=O)CC1] | 317.41 | 40 mg/ml in DMSO 80 mg/ml in NMP 40 mg/ml in MeOH 20 mg/ml in EtOH 100 mM in | KD = 61.7 ± 5.6 nM |
| | | | | DMSO | |
| 1,2, Benziso- thiazol-3- one; BIT fragment | BIT2 | 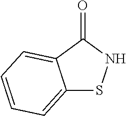 | 151.18 | 100 mM in DMSO | KD = 142.5 ± 21.7 nM |
| Ebselen | | 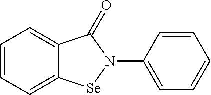 [SMILES: O=c1c2ccccc2[se]n1-c1ccccc1] | 274.18 | Poor | two binding site; KD1 = 5.2 ± 3.9 nM; KD2 = 111.2 ± 45.0 nM |
| LC-1 | MMG 832A | 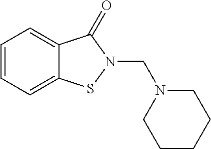 [SMILES: O=c1c2ccccc2sn1CN1CCCCC1] | 248.1 | 100 mM in DMSO | KD = 107.3 ± 14.4 nM |
| LC-2 | MMG 832B | 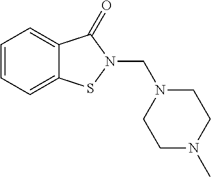 [SMILES: CN1CCN(Cn2sc3ccccc3c2=O)CC1] | 263.36 | 100 mM in DMSO | KD = 133.9 ± 20.4 nM |
| LC-3 | MMG 832C | 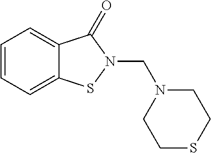 [SMILES: O=c1c2ccccc2sn1CN1CCSCC1] | 266.05 | 100 mM in DMSO | KD = 91.7 ± 15.9 nM |
| LC-4 | MMG 832D | 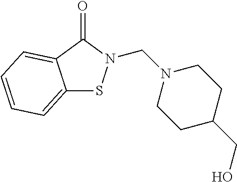 [SMILES: O=c1c2ccccc2sn1CN1CCC(CO)CC1] | 278.37 | 100 mM in DMSO | KD = 265.3 ± 36.0 nM |
| LC-5 | PHB-5011 | 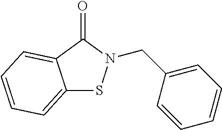 [SMILES: O=c1c2ccccc2sn1Cc1ccccc1] | 241.31 | 100 mM in DMSO | KD1 = 42.7 ± 36.5 nM; KD2 = 2267.6 ± 1511.7 nM |
| LC-6 | PHB-5012 | 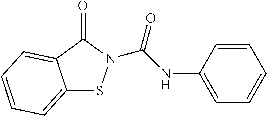 [SMILES: O=C(Nc1ccccc1)n1sc2ccccc2c1=O] | 270.31 | 50 mM in DMSO | KD = not active |
| LC-7 | MMG 836C | 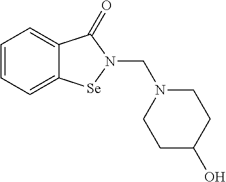 [SMILES: O=c1c2ccccc2[se]n1CN1CCC(O)CC1] | 264.3 | 100 mM in DMSO | KD = 216.0 ± 30.5 nM |
| LC-8 | MMG 836D | 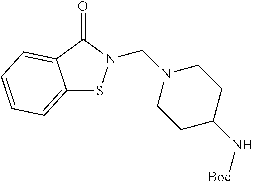 [SMILES: CC(C)(C)OC(=O)NC1CCN(Cn2sc3ccccc3c2=O)CC1] | 363.48 | 50 mM in DMSO | KD = 94.3 ± 20.4 nM |
| LC-9 | AZII67A | 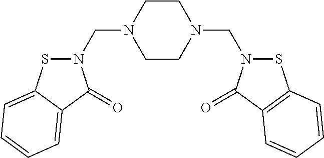 [SMILES: O=c1c2ccccc2sn1CN1CCN(Cn2sc3ccccc3c2=O)CC1] | 412.5 | poor, <6.75 mM in DMSO | poor solubility |
| LC-10 | AZII65 | 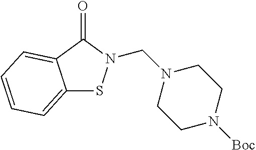 [SMILES: CC(C)(C)OC(=O)N1CCN(Cn2sc3ccccc3c2=O)CC1] | 287.14 | 100 mM in DMSO | KD = 123.3 ± 33.6 nM |
| LC-12 | MMG838 A | 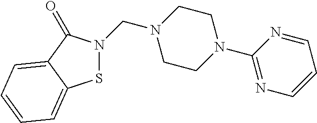 [SMILES: O=c1c2ccccc2sn1CN1CCN(c2ncccn2)CC1] | 327.41 | 100 mM in DMSO | KD = 140.3 ± 28.5 nM |
| LC-13 | MMG838 B | 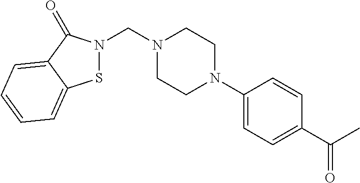 [SMILES: CC(=O)c1ccc(N2CCN(Cn3sc4ccccc4c3=O)CC2)cc1] | 367.14 | 100 mM in DMSO | KD = 189.8 ± 26.9 nM |
| LC-15 | AZII74 | 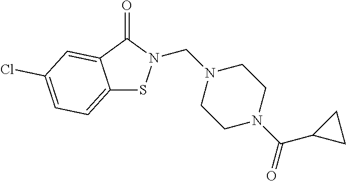 [SMILES: O=C(C1CC1)N1CCN(Cn2sc3ccc(Cl)cc3c2=O)CC1] | 351.8 | 20 mM in DMSO | KD = 113.6 ± 17.3 nM |

### Example 13

### Design and Evaluation of Additional LOC14 Analogs

Additional LOC14 analogs were designed and synthesized as described in the synthetic schemes or as would be known by one skilled in the art. The analogs were evaluated for their binding to PDIa using isothermal titration calorimetry (ITC). The analogs were further evaluated using an Insulin Assay (either active or inactive) and a Cell Viability Assay. Next, the analogs were tested for their ability to rescue PC12 cells from mHTT^{Q103} induced cell death in a dose-dependent manner. The data are presented in Table 5 below.

TABLE 5
Analysis of LOC14 Analogs (Complete List).

| | | | | | Insulin | | Cell Viability |
|---|---|---|---|---|---|---|---|
| Our ID | Name | Structure | MW | Solubility | Assay | ITC Assay | Assay |
| LOC14 | MMG524 | 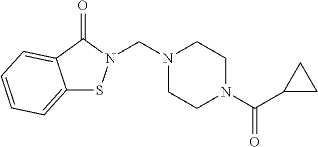 [SMILES: O=C(C1CC1)N1CCN(Cn2sc3ccccc3c2=O)CC1] | 317.41 | 100 mM in DMSO | Active | KD = 113.4 ± 43.4 nM | EC50 = 101.7 nM |
| LC-1 | MMG 832A | 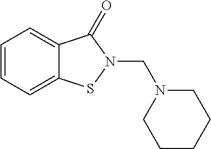 [SMILES: O=c1c2ccccc2sn1CN1CCCCC1] | 248.1 | 100 mM in DMSO | Active | KD = 107.3 ± 14.4 nM | EC50 = 459 nM |
| LC-2 | MMG 832B | 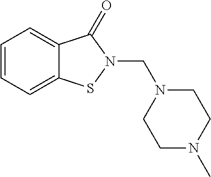 [SMILES: CN1CCN(Cn2sc3ccccc3c2=O)CC1] | 263.36 | 100 mM in DMSO | Active | KD = 91.7 ± 20.4 nM | EC50 = 414.9 nM |
| LC-3 | MMG 832C | 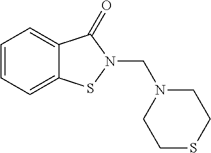 [SMILES: O=c1c2ccccc2sn1CN1CCSCC1] | 266.05 | 100 mM in DMSO | Active | KD = 91.7 ± 15.9 nM | EC50 = 462.7 nM |
| LC-4 | MMG 832D | 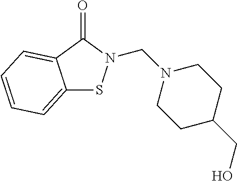 [SMILES: O=c1c2ccccc2sn1CN1CCC(CO)CC1] | 278.37 | 100 mM in DMSO | Active | KD = 265.3 ± 36.0 nM | EC50 = 342.6 nM |
| LC-5 | PHB-5011 | 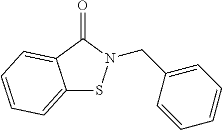 [SMILES: O=c1c2ccccc2sn1Cc1ccccc1] | 241.31 | 100 mM in DMSO | Active | 2 binding sites KD1 = 42.7 ± 36.5 nM; KD2 = 2267.6 ± 1611.7 nM | EC50 = 768.3 nM |
| LC-6 | PHB-5012 | 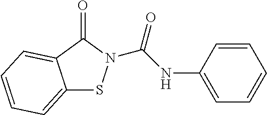 [SMILES: O=C(Nc1ccccc1)n1sc2ccccc2c1=O] | 270.31 | 30 mM in DMSO | not active | KD = N/A irreversible or not active | EC50 = 476.3 nM |
| LC-7 | MMG 836C | 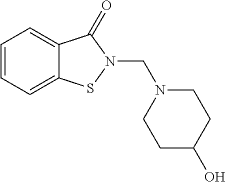 [SMILES: O=c1c2ccccc2sn1CN1CCC(O)CC1] | 264.3 | 100 mM in DMSO | Active | KD = 216.0 ± 30.5 nM | EC50 = 441.1 nM |
| LC-8 | MMG 836D | 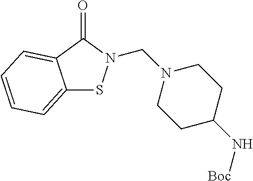 [SMILES: CC(C)(C)OC(=O)NC1CCN(Cn2sc3ccccc3c2=O)CC1] | 363.48 | 50 mM in DMSO | Active | KD = 94.3 ± 20.4 nM | EC50 = 383.8 nM |
| LC-9 | AZII67A | 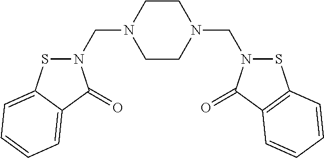 [SMILES: O=c1c2ccccc2sn1CN1CCN(Cn2sc3ccccc3c2=O)CC1] | 412.5 | 5 mM in DMSO | Active | poor solubility | EC50 = 173.0 nM |
| LC-10 | AZII65 | 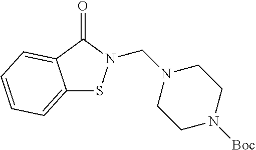 [SMILES: CC(C)(C)OC(=O)N1CCN(Cn2sc3ccccc3c2=O)CC1] | 287.14 | 100 mM in DMSO | Active | KD = 123.3 ± 33.6 nM | EC50 = 580.7 nM |
| LC-11 | PHB-5007 | 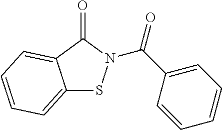 [SMILES: O=C(c1ccccc1)n1sc2ccccc2c1=O] | 255.29 | 100 mM in DMSO | not active | KD = N/A irreversible or not | EC50 >50,000 nM |
| LC-12 | MMG838A | 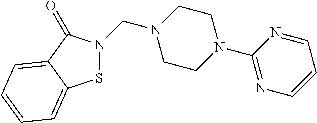 [SMILES: O=c1c2ccccc2sn1CN1CCN(c2ncccn2)CC1] | 327.41 | 100 mM in DMSO | Active | KD = 140.3 ± 28.5 nM | EC50 = 415.7 nM |
| LC-13 | MMG838B | 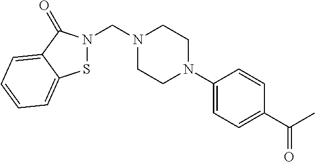 [SMILES: CC(=O)c1ccc(N2CCN(Cn3sc4ccccc4c3=O)CC2)cc1] | 367.14 | 100 mM in DMSO | Active | KD = 189.6 ± 26.9 nM | EC50 = 513.6 nM |
| LC-14 | PHB-6013 PHB5028 | 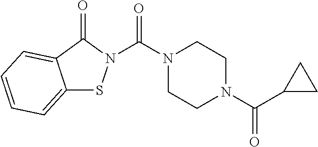 [SMILES: O=C(C1CC1)N1CCN(C(=O)n2sc3ccccc3c2=O)CC1] | 331.39 | 30 mM in DMSO | not active | KD = N/A irreversible or not active | EC50 >50,000 nM |
| LC-15 | AZII74 | 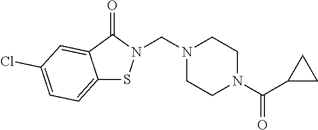 [SMILES: O=C(C1CC1)N1CCN(Cn2sc3ccc(Cl)cc3c2=O)CC1] | 351.8 | 15 mM in DMSO | Active | KD = 113.6 ± 17.3 nM | EC50 = 467.3 nM |
| LC-16 | AZII84T | 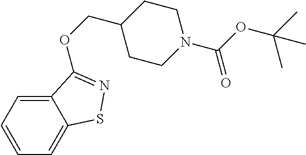 [SMILES: CC(C)(C)OC(=O)N1CCC(COc2nsc3ccccc23)CC1] | 348.5 | 50 mM in DMSO | not active | KD = N/A irreversible or not active | EC50 >50,000 nM |
| LC-17 | AZII83 | 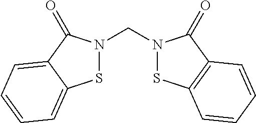 [SMILES: O=c1c2ccccc2sn1Cn1sc2ccccc2c1=O] | 314.4 | 100 mM in DMSO | Active | KD = 160.0 ± 43.0 nM | EC50 = 229.4 nM |
| LC-18 | MMG649A | 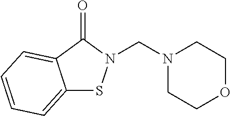 [SMILES: O=c1c2ccccc2sn1CN1CCOCC1] | 250.3 | 100 mM in DMSO | Active | KD = 253.2 ± 42.8 nM | EC50 = 546.9 nM |
| LC-19 | PHB-5019 | 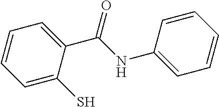 [SMILES: O=C(Nc1ccccc1)c1ccccc1S] | 229.3 | 50 mM in DMSO | not active | KD = N/A irreversible or not active | EC50 = 1,635 nM |
| LC-20 | PHB-5020 | 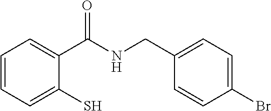 [SMILES: O=C(NCc1ccc(Br)cc1)c1ccccc1S] | 322.22 | 100 mM in DMSO | not active t | KD = N/A irreversible or not active | EC50 >50,000 nM |
| LC-21 | PHB-5021 | 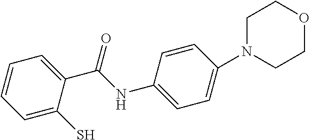 [SMILES: O=C(Nc1ccc(N2CCOCC2)cc1)c1ccccc1S] | 314.4 | 100 mM in DMSO | not active | KD = N/A irreversible or not active | EC50 = 695.7 nM |
| LC-22 | PHB-5022 | 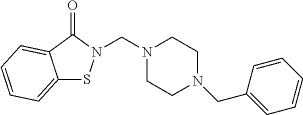 [SMILES: O=c1c2ccccc2sn1CN1CCN(Cc2ccccc2)CC1] | 339.46 | 100 mM in DMSO | Active | KD = 173.3 ± 32.4 nM | EC50 = 309.8 nM |
| LC-23 | PHB-5024 | 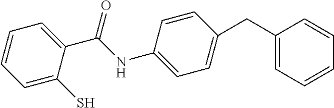 [SMILES: O=C(Nc1ccc(Cc2ccccc2)cc1)c1ccccc1S] | 319.42 | 100 mM in DMSO | not active | KD = N/A irreversible or not active | EC50 = 3,883 nM |
| LC-24 | PHB-5039 | 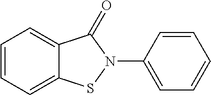 [SMILES: O=c1c2ccccc2sn1-c1ccccc1] | 227.28 | 50 mM in DMSO | not active | 2 binding sites KD1 = 8.1 ± 6.6 nM KD2 = 1137.7 ± 529.4 nM | EC50 = 1,134 nM |
| LC-25 | PHB-5040 | 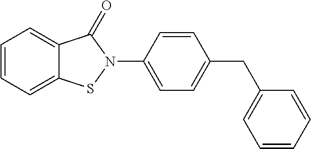 [SMILES: O=c1c2ccccc2sn1-c1ccc(Cc2ccccc2)cc1] | 317.41 | 100 mM in DMSO | not active | KD = N/A irreversible or not active | EC50 = 1,591 nM |
| LC-26 | PHB-5041 | 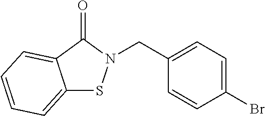 [SMILES: O=c1c2ccccc2sn1Cc1ccc(Br)cc1] | 320.2 | 100 mM in DMSO | not active | KD = N/A irreversible or not active | EC50 = 778.1 nM |
| LC-27 | PHB-5042 | 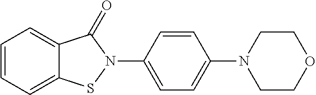 [SMILES: O=c1c2ccccc2sn1-c1ccc(N2CCOCC2)cc1] | 312.39 | 30 mM in DMSO | not active | KD = N/A irreversible or not active | EC50 = 1,828 nM |
| LC-28 | PHB-5047 | 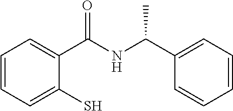 [SMILES: C[C@@H](NC(=O)c1ccccc1S)c1ccccc1] | 257.35 | 100 mM in DMSO | not active | KD = N/A irreversible or not active | EC50 = 808 nM |
| LC-29 | PHB-5052 | 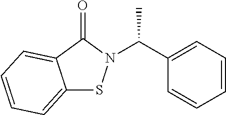 [SMILES: C[C@H](c1ccccc1)n1sc2ccccc2c1=O] | 255.34 | 100 mM in DMSO | Active | KD = 657.9 ± 223.8 nM | EC50 = 518.8 nM |
| LC-30 | PHB-5048 | 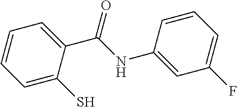 [SMILES: O=C(Nc1cccc(F)c1)c1ccccc1S] | 247.29 | 100 mM in DMSO | not active | KD = N/A irreversible or not active | EC50 = 2,992 nM |
| LC-31 | PHB-5053 | 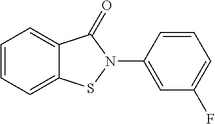 [SMILES: O=c1c2ccccc2sn1-c1cccc(F)c1] | 245.27 | 100 mM in DMSO | not active | KD = N/A irreversible or not | EC50 = 1,765 nM |
| LC-32 | PHB-5045 | 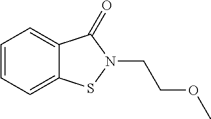 | 209.26 | 100 mM in DMSO | Active | KD 158.2 ± 60.8 nM | EC50 = 700.1 nM |
| LC-33 | PHB-5049 | 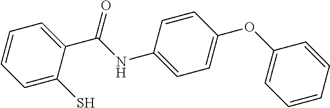 [SMILES: O=C(Nc1ccc(Oc2ccccc2)cc1)c1ccccc1S] | 321.39 | 100 mM in DMSO | not active | KD = N/A irreversible or not active | EC50 = 2,937 nM |
| LC-34 | PHB-5051 | 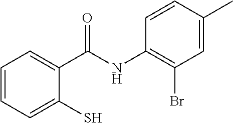 [SMILES: Cc1ccc(NC(=O)c2ccccc2S)c(Br)c1] | 322.22 | 100 mM in DMSO | not active | KD = N/A irreversible or not active | EC50 = 3,767 nM |
| LC-35 | MI | 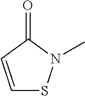 | 115.15 | 100 mM in DMSO | Active | KD = N/A irreversible or not active | Not Active |
| LC-36 | PHB-5055 | 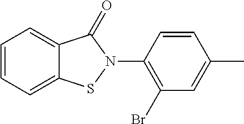 [SMILES: Cc1ccc(-n2sc3ccccc3c2=O)c(Br)c1] | 320.2 | 100 mM in DMSO | not active | KD = 25.3 ± 10.7 nM | EC50 = 1,594 nM |
| LC-37 | PHB-5056 | 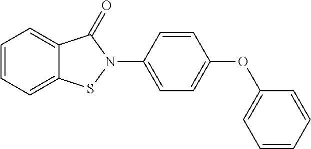 [SMILES: O=c1c2ccccc2sn1-c1ccc(Oc2ccccc2)cc1] | 319.38 | 100 mM in DMSO | not active | KD = N/A irreversible or not active | EC50 = 1,679 nM |
| LC-38 | AZIII24 | 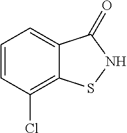 [SMILES: O=c1[nH]sc2c(Cl)cccc12] | 185.6 | 100 mM in DMSO | not active | KD = N/A irreversible or not active | Not Active |
| LC-39 | AZIII26C | 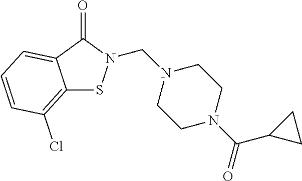 [SMILES: O=C(C1CC1)N1CCN(Cn2sc3c(Cl)cccc3c2=O)CC1] | 351.8 | 100 mM in DMSO | not active | KD = N/A irreversible or not active | Not Active |
| LC-40 | AZIII33 | 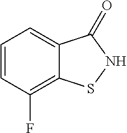 [SMILES: O=c1[nH]sc2c(F)cccc12] | 169.2 | 100 mM in DMSO | Active | KD = N/A irreversible or not active | EC50 = 9,325 nM |
| LC-41 | AZIII32 | 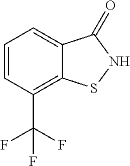 [SMILES: O=c1[nH]sc2c(C(F)(F)F)cccc12] | 219.2 | 100 mM in DMSO | not active | KD = N/A irreversible or not active | Not Active |
| LC-42 | AZIII34 | 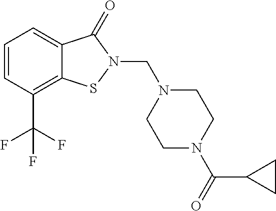 [SMILES: O=C(C1CC1)N1CCN(Cn2sc3c(C(F)(F)F)cccc3c2=O)CC1] | 385.4 | 100 mM in DMSO | not active | KD = N/A irreversible or not active | Not Active |
| LC-43 | AZIII35 | 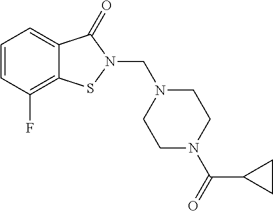 [SMILES: O=C(C1CC1)N1CCN(Cn2sc3c(F)cccc3c2=O)CC1] | 335.4 | 100 mM in DMSO | Active | KD = N/A irreversible or not active | EC50 = 7,409 nM |
| LC-44 | AZIII29 | 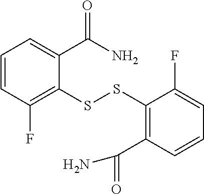 [SMILES: NC(=O)c1cccc(F)c1SSc1c(F)cccc1C(N)=O] | 340.4 | 100 mM in DMSO | Active | KD = N/A irreversible or not active | EC50 = 7,066 nM |
| LC-45 | AZIII28 | 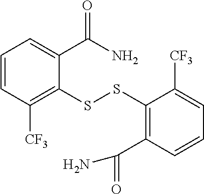 [SMILES: NC(=O)c1cccc(C(F)(F)F)c1SSc1c(C(N)=O)cccc1C(F)(F)F] | 440.4 | 100 mM in DMSO | not active | KD = N/A irreversible or not active | Not Active |
| LC-46 | PHB-5062 | 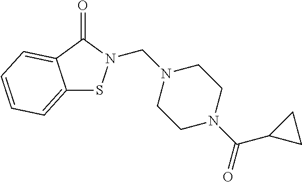 [SMILES: O=C(C1CC1)N1CCN(Cn2sc3ccccc3c2=O)CC1] | 318.4 | 50 mM in DMSO | Active | KD = 9.6 ± 3.2 nM | EC50 = 2.920 nM |
| LC-47 | PHB-5063 | 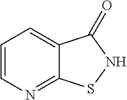 | 152.17 | 50 mM in DMSO | not active | KD = N/A irreversible or not active | EC50 >50,000 nM |
| LC-48 | AZIII60A | 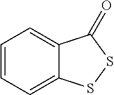 | 166.2 | 100 mM in DMSO | not active | N/A | Not Active |
| LC-49 | AZIII60B | 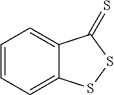 | 184.3 | 100 mM in DMSO | not active | N/A | Not Active |
| LC-50 | AZIII60D | 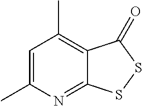 | 180.2 | 100 mM in DMSO | Active | N/A | EC50 = 5,180 nM |
| LC-51 | AZIII60C | 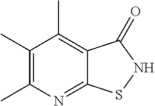 | 194.3 | 10 mM in DMSO | Active | N/A | EC50 = 2,200 nM |
| LC-52 | AZIII92A | 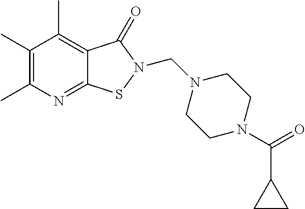 [SMILES: Cc1nc2sn(CN3CCN(C(=O)C4CC4)CC3)c(=O)c2c(C)c1C] | 360.5 | 10 mM in DMSO | Active | N/A | EC50 = 3,570 nM |
| LC-53 | AZIII93 | 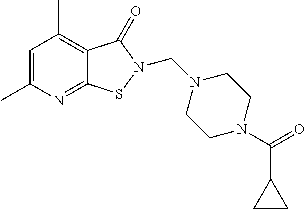 [SMILES: Cc1cc(C)c2c(=O)n(CN3CCN(C(=O)C4CC4)CC3)sc2n1] | 346.4 | 25 mM in DMSO | Active | N/A | EC50 = 4,200 nM |
| LC-54 | PHB5077 | 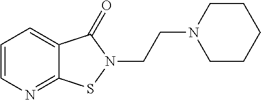 [SMILES: O=c1c2cccnc2sn1CCN1CCCCC1] | 263.36 | 100 mM in DMSO | Active | N/A | Not Active |
| LC-55 | PHB5078 | 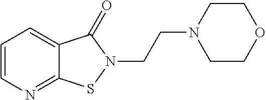 [SMILES: O=c1c2cccnc2sn1CCN1CCOCC1] | 265.33 | 100 mM in DMSO | Active | N/A | Not Active |
| LC-56 | PHB5079 | 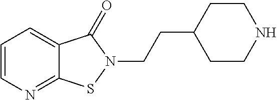 [SMILES: O=c1c2cccnc2sn1CCC1CCNCC1] | 263.36 | 100 mM in DMSO | Active | N/A | Not Active |
| LC-57 | PHB5088 | 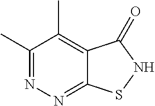 | 161.21 | 100 mM in DMSO | Active | N/A | EC50 = 12,150 nM |
| LC-58 | PHB5084 | 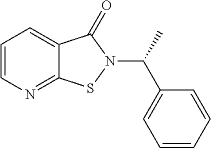 [SMILES: C[C@H](c1ccccc1)n1sc2ncccc2c1=O] | 256.32 | 100 mM in DMSO | Active | N/A | Not Active |
| LC-59 | PHB5089 | 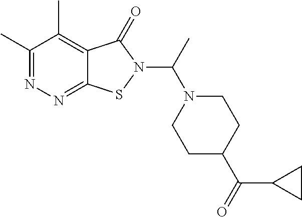 [SMILES: Cc1nnc2sn(C(C)N3CCC(C(=O)C4CC4)CC3)c(=O)c2c1C] | 347.44 | 100 mM in DMSO | Active | N/A | EC50 = 10,600 nM |

* N/A: data not available at this time

### Example 14

### LOC14 and its Analogs are Reversible, Neuroprotective, Nanomolar Modulators of PDI

In this study, LOC14 and its analogs were identified and characterized as the first reversible, neuroprotective, nanomolar modulators of PDI. It was found that LOC14 reversibly binds to a region adjacent to the active site of PDI, induces the protein to adopt an oxidized conformation, and inhibits its reductase activity. A possible mechanism of inhibition is shown in FIG. 11. It was found that the oxidation of PDI by LOC14 is protective in PC12 cells and in medium spiny neurons that degenerate from transfected mutant huntingtin protein expression. Furthermore, LOC14 displayed high in vitro metabolic stability in mouse liver microsomes and blood plasma, making it a promising candidate for in vivo mouse studies of PDI's role in protein misfolding diseases.

This is the first report that oxidation of PDI activity is neuroprotective. One possible explanation has to do with PDI's binding protein ER oxidoreductin 1 (Ero1). During protein folding, PDI cycles between oxidized and reduced disulfide states. When it forms a disulfide bond with a substrate protein, its own catalytic site becomes reduced. In vitro, PDI can be oxidized by GSSG, but in vivo the protein Ero1 is needed to accomplish this task. Ero1 is a flavin-adenine-dinucleotide-(FAD)-bound protein that takes electrons from re-oxidized PDI and passes them onto molecular oxygen as a terminal acceptor, in the process creating hydrogen peroxide and thus generating reactive oxygen species (ROS). By oxidizing PDI with LOC14, Ero1 can be bypassed, reducing the generation of ROS and hence providing neuroprotection. This is consistent with previous findings that reported that overexpression of PDI is toxic in neuron-like PC12 cells, but can be protected from this overexpression toxicity with the irreversible PDI inhibitor 16F16 (Hoffstrom et al., 2010). By increasing the level of PDI production, one would increase Ero1 oxidation of PDI, leading to increased ROS generation. It is possible that irreversible PDI inhibitors covalently bound to the active site cysteines, prevent the Ero1-PDI interaction, and thus oxidation.

To our knowledge, this is the first compound reported to date that binds reversibly to PDI with low nanomolar affinity and causes protection in neuronal cells and tissue. Inhibition of PDI activity causing neuroprotection has not been validated yet in in vivo mouse models of neurodegeneration, due to the lack of drug-like inhibitors with low cytotoxic properties. Previously reported PDI inhibitors that are cell permeable bind covalently and irreversibly to PDI. Ultimately, this type of binding completely inactivates the protein, can be non-selective, and can result in haptenization, causing unpredicted idiosyncratic toxicity from the immune system in vivo leading to liver failure and blood disorders. The reversible modulation of PDI with LOC14 overcomes these challenges. LOC14 forms covalent, but reversible, bonds with the protein, ultimately acting like a non-covalent inhibitor (because of its potent, but reversible, effects on the protein). Thus, in vivo, LOC14 may not result in idiosyncratic toxicities. Furthermore, LOC14 showed high stability in liver microsomes and blood plasma, making it a promising candidate for future in vivo work.

The catalytic a domain of PDI A1 was used as a prototype of the redox reactions that the PDI family of proteins catalyze. The N-terminal cysteine of the a domain in PDI A1 is less reactive than the N-terminal cysteine of the a′ domain of PDI A1, and both have lower hyper-activity than the catalytic cysteines in PDI A3 (ERp57). Thus, it is very likely that LOC14 will react and oxidize both catalytic domains of PDI A1 and PDI A3. Previously, it was reported that both PDI A3 and PDI A1 proteins (Hoffstrom et al., 2010) (and possibly PDI A4 and PDI A6 (Ge et al., 2013)) were the target of 16F16. Since numerous PDI family members reside in the ER and their distinct roles are still unclear, it is possible that modulation of the whole family by LOC14 is neuroprotective.

In summary, a new scaffold, LOC14, was identified for reversible inhibition of PDI's reductase activity. This compound, although targeting similar residues of PDI as the irreversible inhibitor 16F16, forces the protein to adopt a different conformation that resembles the native oxidized form. LOC14 has improved solubility, potency and in vitro metabolism properties compared to other reported PDI inhibitors, and it protects neuron-like PC12 cells as well as bona fide striatal MSNs from mutant huntingtin toxicity. Validating PDI as a target for neurodegenerative disorders may open new therapeutic strategies to treat and understand these diseases.

### DOCUMENTS

Aiken C T, Tobin A J, & Schweitzer E S (2004) A cell-based screen for drugs to treat Huntington's disease. Neurobiol Dis 16(3):546-555.
Atkin J D, et al. (2008) Endoplasmic reticulum stress and induction of the unfolded protein response in human sporadic amyotrophic lateral sclerosis. Neurobiol Dis 30(3):400-407.
Barbouche R, Miquelis R, Jones I M, & Fenouillet E (2003) Protein-disulfide isomerase-mediated reduction of two disulfide bonds of HIV envelope glycoprotein 120 occurs post-CXCR4 binding and is required for fusion. J Biol Chem 278(5):3131-3136.
Cantley A M, et al. (2014) Small Molecule that Reverses Dexamethasone Resistance in T-cell Acute Lymphoblastic Leukemia (T-ALL). ACS Med Chem Lett 5(7):754-759.
Cardinale C J, et al. (2008) Termination factor Rho and its cofactors NusA and NusG silence foreign DNA in E. coli. Science 320(5878):935-938.
Cho J, Furie B C, Coughlin S R, & Furie B (2008) A critical role for extracellular protein disulfide isomerase during thrombus formation in mice. J Clin Invest 118(3):1123-1131.
Colla E, et al. (2012) Endoplasmic reticulum stress is important for the manifestations of alpha-synucleinopathy in vivo. J Neurosci 32(10):3306-3320.
Darby N J & Creighton T E (1995) Functional properties of the individual thioredoxin-like domains of protein disulfide isomerase. Biochemistry 34(37):11725-11735. Dixon S J, et al. (2012) Ferroptosis: an iron-dependent form of nonapoptotic cell death. Cell 149(5):1060-1072.
Fuchslueger U, Socher G, Grether H J, & Grasserbauer M (1999) Capillary supercritical fluid chromatography/mass spectrometry of phenolic mannich bases with dimethyl ether modified ethane as the mobile phase. Anal Chem 71(13):2324-2333.
Garbi N, Tanaka S, Momburg F, & Hammerling G J (2006) Impaired assembly of the major histocompatibility complex class I peptide-loading complex in mice deficient in the oxidoreductase ERp57. Nat Immunol 7(1):93-102.
Ge J, et al. (2013) Small molecule probe suitable for in situ profiling and inhibition of protein disulfide isomerase. ACS Chem Biol 8(11):2577-2585.
Hashida T, Kotake Y, & Ohta S (2011) Protein disulfide isomerase knockdown-induced cell death is cell-line-dependent and involves apoptosis in MCF-7 cells. J Toxicol Sci 36(1):1-7.
Hoffstrom B G, et al. (2010) Inhibitors of protein disulfide isomerase suppress apoptosis induced by misfolded proteins. Nat Chem Biol 6(12):900-906.
Holla B S, Shivananda M K, Shenoy M S, & Antony G (1998) Studies on arylfuran derivatives. Part VII. Synthesis and characterization of some Mannich bases carrying halophenylfuryl moieties as promising antibacterial agents. Farmaco 53(8-9):531-535.
Idhayadhulla A, Surendra Kumar R, Abdul Nasser A J, & Manilal A (2011) Synthesis and antimicrobial activity of some new Mannich base derivatives. J. Chem. Pharm. Res. 3(4):904-911.
Jasuja R, et al. (2012) Protein disulfide isomerase inhibitors constitute a new class of antithrombotic agents. J Clin Invest 122(6):2104-2113.
Karala A R & Ruddock L W (2010) Bacitracin is not a specific inhibitor of protein disulfide isomerase. FEBS J 277(11):2454-2462.
Kemmink J, Darby N J, Dijkstra K, Scheek R M, & Creighton T E (1995) Nuclear magnetic resonance characterization of the N-terminal thioredoxin-like domain of protein disulfide isomerase. Protein Science 4(12):2587-2593.
Khan M M, et al. (2011) Discovery of a small molecule PDI inhibitor that inhibits reduction of HIV-1 envelope glycoprotein gp120. ACS Chem Biol 6(3):245-251. Koivu J, et al. (1987) A single polypeptide acts both as the beta subunit of prolyl 4-hydroxylase and as a protein disulfide-isomerase. J Biol Chem 262(14):6447-6449.
Lipinski C A, Lombardo F, Dominy B W, & Feeney P J (2001) Experimental and computational approaches to estimate solubility and permeability in drug discovery and development settings. Adv Drug Deliv Rev 46(1-3):3-26.
Lovat P E, et al. (2008) Increasing melanoma cell death using inhibitors of protein disulfide isomerases to abrogate survival responses to endoplasmic reticulum stress. Cancer Res 68(13):5363-5369.
Reinhart P H, et al. (2011) Identification of anti-inflammatory targets for Huntington's disease using a brain slice-based screening assay. Neurobiol Dis 43(1):248-256.
Smith A M, et al. (2004) A high-throughput turbidometric assay for screening inhibitors of protein disulfide isomerase activity. J Biomol Screen 9(7):614-620.
Suhr S T, Gil E B, Senut M C, & Gage F H (1998) High level transactivation by a modified Bombyx ecdysone receptor in mammalian cells without exogenous retinoid X receptor. Proc Natl Acad Sci USA 95(14):7999-8004.
Vilaboa N, Boellmann F, & Voellmy R (2011) Gene Switches for Deliberate Regulation of Transgene Expression: Recent Advances in System Development and Uses. Journal of genetic syndrome & gene therapy 2(3): 107.
Wetterau J R, Combs K A, Spinner S N, & Joiner B J (1990) Protein disulfide isomerase is a component of the microsomal triglyceride transfer protein complex. J Biol Chem 265(17):9800-9807.
Williamson M P (2013) Using chemical shift perturbation to characterise ligand binding. Prog Nucl Magn Reson Spectrosc 73:1-16.
Xu S, et al. (2012) Discovery of an orally active small-molecule irreversible inhibitor of protein disulfide isomerase for ovarian cancer treatment. Proc Natl Acad Sci USA 109(40):16348-16353.
Yang W S, et al. (2014) Regulation of ferroptotic cancer cell death by GPX4. Cell 156(1-2):317-331.
Yoo B C, et al. (2002) Overexpressed protein disulfide isomerase in brains of patients with sporadic Creutzfeldt-Jakob disease. Neurosci Lett 334(3):196-200.
Zhang J H, Chung T D, & Oldenburg K R (1999) A Simple Statistical Parameter for Use in Evaluation and Validation of High Throughput Screening Assays. J Biomol Screen 4(2):67-73.

All patents, patent applications, and publications cited above are incorporated herein by reference in their entirety as if recited in full herein.

The invention being thus described, it will be obvious that the same may be varied in many ways. Such variations are not to be regarded as a departure from the spirit and scope of the invention and all such modifications are intended to be included within the scope of the following claims.

What is claimed is:

1. A method for treating or ameliorating the effects of a neurodegenerative disorder in a subject in need thereof comprising administering to the subject an effective amount of a compound selected from the group consisting of: 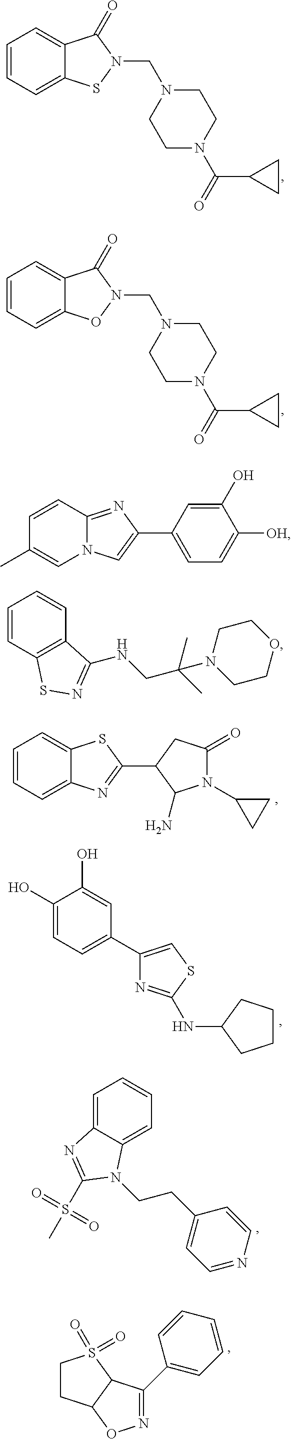 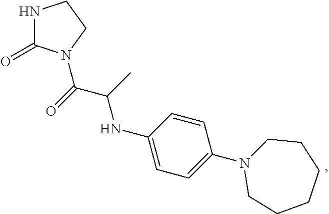 [SMILES: CC(Nc1ccc(N2CCCCCC2)cc1)C(=O)N1CCNC1=O]
combinations thereof,
or an N-oxide, crystalline form, hydrate thereof, or a pharmaceutically acceptable salt thereof.

2. The method of claim 1, wherein the neurodegenerative disease is selected from the group consisting of Alzheimer's disease, Parkinson's disease, amyotrophic lateral sclerosis (ALS), Friedreich's ataxia, multiple sclerosis, Huntington's Disease, transmissible spongiform encephalopathy, Charcot-Marie-Tooth disease, dementia with Lewy bodies, corticobasal degeneration, progressive supranuclear palsy, and hereditary spastic paraparesis.

3. The method of claim 1, wherein the neurodegenerative disease is Huntington's Disease.

4. The method of claim 1, wherein the compound is 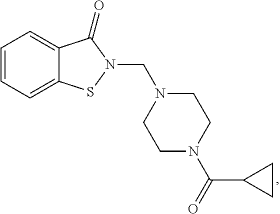 [SMILES: O=C(C1CC1)N1CCN(Cn2sc3ccccc3c2=O)CC1]
or an N-oxide, crystalline form, hydrate thereof, or a pharmaceutically acceptable salt thereof.

5. The method of claim 1, wherein the subject is a mammal.

6. The method of claim 5, wherein the mammal is selected from the group consisting of humans, veterinary animals, and agricultural animals.

7. The method of claim 1, wherein the subject is a human.

8. The method of claim 1, further comprising co-administering to the subject an effective amount of one or more additional therapeutic agents.

9. The method of claim 8, wherein the one or more additional therapeutic agents are selected from the group consisting of 5-hydroxytryptophan, Activase, AFQ056 (Novartis), Aggrastat, Albendazole, alpha-lipoic acid/L-acetyl carnitine, Alteplase, Amantadine (Symmetrel), amlodipine, Ancrod, Apomorphine (Apokyn), Arimoclomol, Arixtra, Armodafinil, Ascorbic acid, Ascriptin, Aspirin, atenolol, Avonex, baclofen (Lioresal), Banzel, Benztropine (Cogentin), Betaseron, BGG492 (Novartis Corp.), Botulinum toxin, Bufferin, Carbatrol®, Carbidopa/levodopa immediate-release (Sinemet), Carbidopa/levodopa oral disintegrating (Parcopa), Carbidopa/levodopa/Entacapone (Stalevo), CERE-110: Adeno-Associated Virus Delivery of NGF (Ceregene), cerebrolysin, CinnoVex, citalopram, citicoline, Clobazam, Clonazepam, Clopidogrel, clozapine (Clozaril), Coenzyme Q, Creatine, dabigatran, dalteparin, Dapsone, Davunetide, Deferiprone, Depakene®, Depakote ER®, Depakote®, Desmoteplase, Diastat, Diazepam, Digoxin, Dilantin®, Dimebon, dipyridamole, divalproex (Depakote), Donepezil (Aricept), EGb 761, Eldepryl, ELND002 (Elan Pharmaceuticals), Enalapril, enoxaparin, Entacapone (Comtan), epoetin alfa, Eptifibatide, Erythropoietin, Escitalopram, Eslicarbazepine acetate, Esmolol, Ethosuximide, Ethyl-EPA (Miraxion™), Exenatide, Extavia, Ezogabine, Felbamate, Felbatol®, Fingolimod (Gilenya), fluoxetine (Prozac), fondaparinux, Fragmin, Frisium, Gabapentin, Gabitril®, Galantamine, Glatiramer (Copaxone), haloperidol (Haldol), Heparin, human chorionic gonadotropin (hCG), Idebenone, Inovelon®, insulin, Interferon beta 1a, Interferon beta 1b, ioflupane 1231 (DATSCAN®), IPX066 (Impax Laboratories Inc.), JNJ-26489112 (Johnson and Johnson), Keppra®, Klonopin, Lacosamide, L-Alpha glycerylphosphorylcholine, Lamictal®, Lamotrigine, Levetiracetam, liraglutide, Lisinopril, Lithium carbonate, Lopressor, Lorazepam, losartan, Lovenox, Lu AA24493, Luminal, LY450139 (Eli Lilly), Lyrica, Masitinib, Mecobalamin, Memantine, methylprednisolone, metoprolol tartrate, Minitran, Minocycline, mirtazapine, Mitoxantrone (Novantrone), Mysoline®, Natalizumab (Tysabri), Neurontin®, Niacinamide, Nitro-Bid, Nitro-Dur, nitroglycerin, Nitrolingual, Nitromist, Nitrostat, Nitro-Time, Norepinephrine (NOR), Carbamazepine, octreotide, Onfi®, Oxcarbazepine, Oxybutinin chloride, PF-04360365 (Pfizer), Phenobarbital, Phenytek®, Phenytoin, piclozotan, Pioglitazone, Plavix, Potiga, Pramipexole (Mirapex), pramlintide, Prednisone, Primidone, Prinivil, probenecid, Propranolol, PRX-00023 (EPIX Pharmaceuticals Inc.), PXT3003, Quinacrine, Ramelteon, Rasagiline (Azilect), Rebif, ReciGen, remacemide, Resveratrol, Retavase, reteplase, riluzole (Rilutek), Rivastigmine (Exelon), Ropinirole (Requip), Rotigotine (Neupro), Rufinamide, Sabril, safinamide (EMD Serono), Salagen, Sarafem, Selegiline (1-deprenyl, Eldepryl), SEN0014196 (Siena Biotech), sertraline (Zoloft), Simvastatin, Sodium Nitroprussiate (NPS), sodium phenylbutyrate, Stanback Headache Powder, Tacrine (Cognex), Tamoxifen, tauroursodeoxycholic acid (TUDCA), Tegretol®, Tenecteplase, Tenormin, Tetrabenazine (Xenazine), THR-18 (Thrombotech Ltd.), Tiagabine, Tideglusib, tirofiban, tissue plasminogen activator (tPA), tizanidine (Zanaflex), TNKase, Tolcapone (Tasmar), Tolterodine, Topamax®, Topiramate, Trihexyphenidyl (formerly Artane), Trileptal®, ursodiol, Valproic Acid, valsartan, Varenicline (Pfizer), Vimpat, Vitamin E, Warfarin, Zarontin®, Zestril, Zonegran®, Zonisamide, Zydis selegiline HCL Oral disintegrating (Zelapar), and combinations thereof.

10. A method for treating or ameliorating the effects of a condition associated with increased protein disulfide isomerase (PDI) activity in a subject in need thereof comprising administering to the subject an effective amount of a compound selected from the group consisting of: 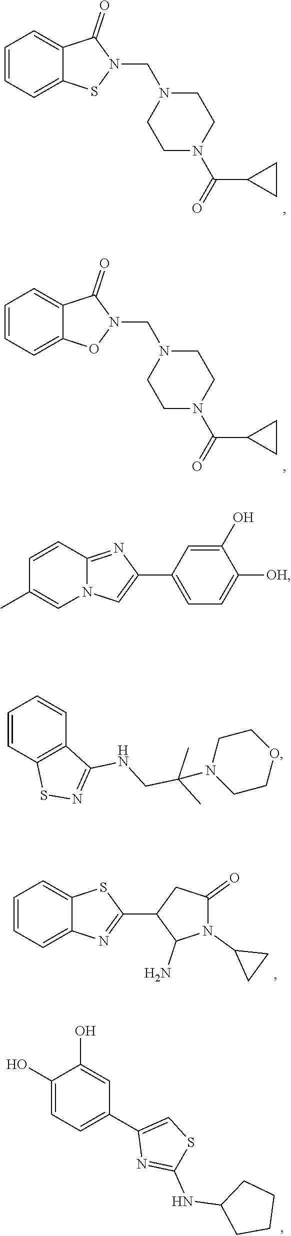 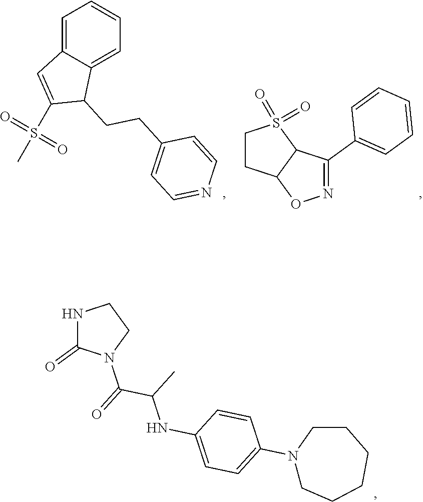
combinations thereof,
or an N-oxide, crystalline form, hydrate thereof, or a pharmaceutically acceptable salt thereof.

11. The method of claim 10, wherein the compound is 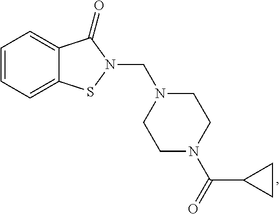 [SMILES: O=C(C1CC1)N1CCN(Cn2sc3ccccc3c2=O)CC1]
or an N-oxide, crystalline form, hydrate thereof, or a pharmaceutically acceptable salt thereof.

12. The method of claim 10, wherein the subject is a mammal.

13. The method of claim 12, wherein the mammal is selected from the group consisting of humans, veterinary animals, and agricultural animals.

14. The method of claim 10, wherein the subject is a human.

15. The method of claim 10, wherein the condition is selected from the group consisting of a protein folding disorder, cancer, HIV, and a blood clot.

16. A method of modulating PDI activity in a cell comprising contacting the cell with an effective amount of a compound selected from the group consisting of: 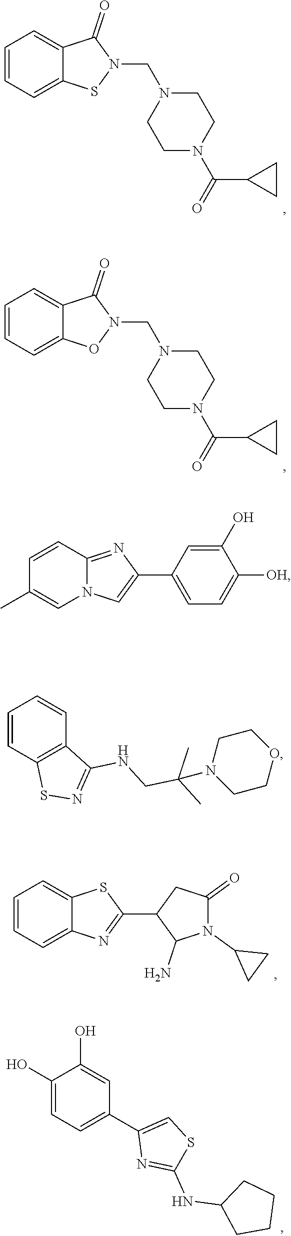 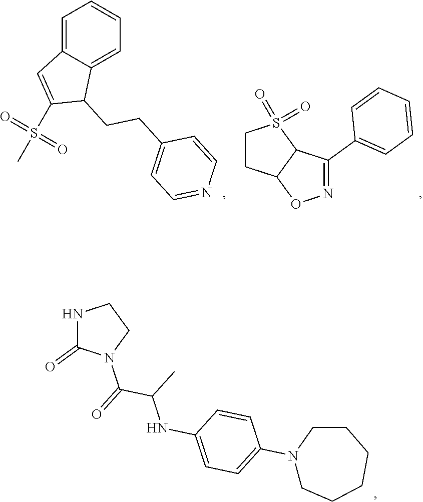
combinations thereof,
or an N-oxide, crystalline form, hydrate thereof, or a pharmaceutically acceptable salt thereof.

17. The method of claim 16, wherein the compound is 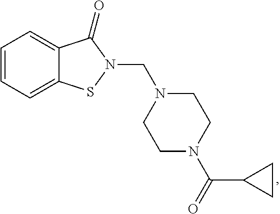 [SMILES: O=C(C1CC1)N1CCN(Cn2sc3ccccc3c2=O)CC1]
or an N-oxide, crystalline form, hydrate thereof, or a pharmaceutically acceptable salt thereof.

18. A method for treating or ameliorating the effects of a neurodegenerative disorder in a subject in need thereof comprising administering to the subject an effective amount of a compound selected from the group consisting of formula (I) 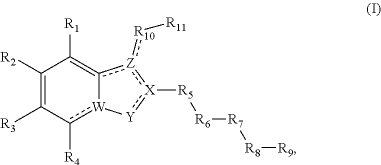
formula (II) 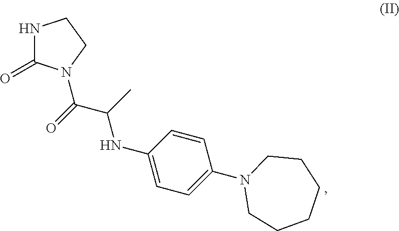 [SMILES: CC(Nc1ccc(N2CCCCCC2)cc1)C(=O)N1CCNC1=O]
formula (III) 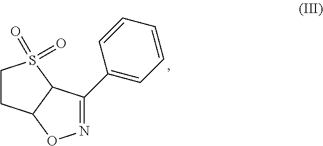 [SMILES: O=S1(=O)CCC2ON=C(c3ccccc3)C21]
and formula (IV): 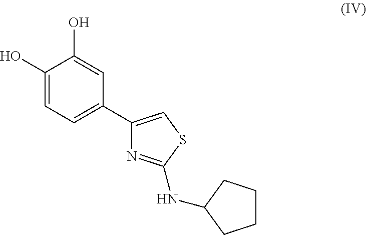 [SMILES: Oc1ccc(-c2csc(NC3CCCC3)n2)cc1O]
wherein a dashed line indicates the presence of an optional double bond, wherein W, X, Y and Z are independently selected from the group consisting of C, N, S and O,
wherein R₁, R₂, R₃, and R₄ are independently selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₅, R₆, R₇, R₈ and R₁₀ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₉ and R₁₁ are independently selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
or an N-oxide, crystalline form, hydrate thereof, or a pharmaceutically acceptable salt thereof.

19. The method of claim 18, wherein the compound is selected from the group consisting of: 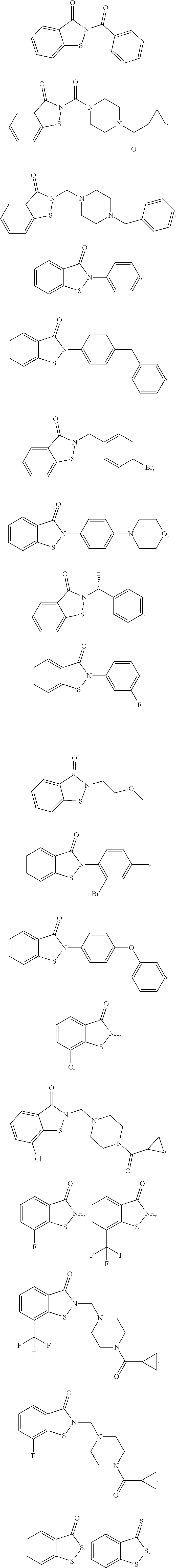
combinations thereof, or an N-oxide, crystalline form, hydrate thereof, or a pharmaceutically acceptable salt thereof.

20. The method of claim 18, wherein the neurodegenerative disease is selected from the group consisting of Alzheimer's disease, Parkinson's disease, amyotrophic lateral sclerosis (ALS), Friedreich's ataxia, multiple sclerosis, Huntington's Disease, transmissible spongiform encephalopathy, Charcot-Marie-Tooth disease, dementia with Lewy bodies, corticobasal degeneration, progressive supranuclear palsy, and hereditary spastic paraparesis.

21. The method of claim 18, wherein the neurodegenerative disease is Huntington's Disease.

22. The method of claim 18, wherein the subject is a mammal.

23. The method of claim 22, wherein the mammal is selected from the group consisting of humans, veterinary animals, and agricultural animals.

24. The method of claim 18, wherein the subject is a human.

25. The method of claim 18, further comprising co-administering to the subject an effective amount of one or more additional therapeutic agents.

26. The method of claim 25, wherein the one or more additional therapeutic agents are selected from the group consisting of 5-hydroxytryptophan, Activase, AFQ056 (Novartis), Aggrastat, Albendazole, alpha-lipoic acid/L-acetyl carnitine, Alteplase, Amantadine (Symmetrel), amlodipine, Ancrod, Apomorphine (Apokyn), Arimoclomol, Arixtra, Armodafinil, Ascorbic acid, Ascriptin, Aspirin, atenolol, Avonex, baclofen (Lioresal), Banzel, Benztropine (Cogentin), Betaseron, BGG492 (Novartis Corp.), Botulinum toxin, Bufferin, Carbatrol®, Carbidopa/levodopa immediate-release (Sinemet), Carbidopa/levodopa oral disintegrating (Parcopa), Carbidopa/levodopa/Entacapone (Stalevo), CERE-110: Adeno-Associated Virus Delivery of NGF (Ceregene), cerebrolysin, CinnoVex, citalopram, citicoline, Clobazam, Clonazepam, Clopidogrel, clozapine (Clozaril), Coenzyme Q, Creatine, dabigatran, dalteparin, Dapsone, Davunetide, Deferiprone, Depakene®, Depakote ER®, Depakote®, Desmoteplase, Diastat, Diazepam, Digoxin, Dilantin®, Dimebon, dipyridamole, divalproex (Depakote), Donepezil (Aricept), EGb 761, Eldepryl, ELND002 (Elan Pharmaceuticals), Enalapril, enoxaparin, Entacapone (Comtan), epoetin alfa, Eptifibatide, Erythropoietin, Escitalopram, Eslicarbazepine acetate, Esmolol, Ethosuximide, Ethyl-EPA (Miraxion™), Exenatide, Extavia, Ezogabine, Felbamate, Felbatol®, Fingolimod (Gilenya), fluoxetine (Prozac), fondaparinux, Fragmin, Frisium, Gabapentin, Gabitril®, Galantamine, Glatiramer (Copaxone), haloperidol (Haldol), Heparin, human chorionic gonadotropin (hCG), Idebenone, Inovelon®, insulin, Interferon beta 1a, Interferon beta 1b, ioflupane 1231 (DATSCAN®), IPX066 (Impax Laboratories Inc.), JNJ-26489112 (Johnson and Johnson), Keppra®, Klonopin, Lacosamide, L-Alpha glycerylphosphorylcholine, Lamictal®, Lamotrigine, Levetiracetam, liraglutide, Lisinopril, Lithium carbonate, Lopressor, Lorazepam, losartan, Lovenox, Lu AA24493, Luminal, LY450139 (Eli Lilly), Lyrica, Masitinib, Mecobalamin, Memantine, methylprednisolone, metoprolol tartrate, Minitran, Minocycline, mirtazapine, Mitoxantrone (Novantrone), Mysoline®, Natalizumab (Tysabri), Neurontin®, Niacinamide, Nitro-Bid, Nitro-Dur, nitroglycerin, Nitrolingual, Nitromist, Nitrostat, Nitro-Time, Norepinephrine (NOR), Carbamazepine, octreotide, Onfi®, Oxcarbazepine, Oxybutinin chloride, PF-04360365 (Pfizer), Phenobarbital, Phenytek®, Phenytoin, piclozotan, Pioglitazone, Plavix, Potiga, Pramipexole (Mirapex), pramlintide, Prednisone, Primidone, Prinivil, probenecid, Propranolol, PRX-00023 (EPIX Pharmaceuticals Inc.), PXT3003, Quinacrine, Ramelteon, Rasagiline (Azilect), Rebif, ReciGen, remacemide, Resveratrol, Retavase, reteplase, riluzole (Rilutek), Rivastigmine (Exelon), Ropinirole (Requip), Rotigotine (Neupro), Rufinamide, Sabril, safinamide (EMD Serono), Salagen, Sarafem, Selegiline (1-deprenyl, Eldepryl), SEN0014196 (Siena Biotech), sertraline (Zoloft), Simvastatin, Sodium Nitroprussiate (NPS), sodium phenylbutyrate, Stanback Headache Powder, Tacrine (Cognex), Tamoxifen, tauroursodeoxycholic acid (TUDCA), Tegretol®, Tenecteplase, Tenormin, Tetrabenazine (Xenazine), THR-18 (Thrombotech Ltd.), Tiagabine, Tideglusib, tirofiban, tissue plasminogen activator (tPA), tizanidine (Zanaflex), TNKase, Tolcapone (Tasmar), Tolterodine, Topamax®, Topiramate, Trihexyphenidyl (formerly Artane), Trileptal®, ursodiol, Valproic Acid, valsartan, Varenicline (Pfizer), Vimpat, Vitamin E, Warfarin, Zarontin®, Zestril, Zonegran®, Zonisamide, Zydis selegiline HCL Oral disintegrating (Zelapar), and combinations thereof.

27. A method of treating or ameliorating the effects of a condition associated with increased protein disulfide isomerase (PDI) activity in a subject in need thereof comprising administering to the subject an effective amount of a compound selected from the group consisting of formula (I) 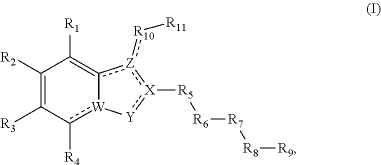
formula (II) 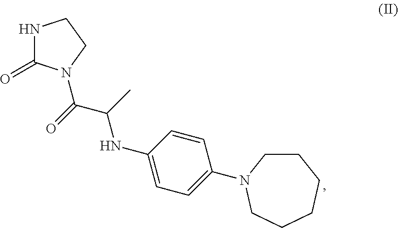 [SMILES: CC(Nc1ccc(N2CCCCCC2)cc1)C(=O)N1CCNC1=O]
formula (III) 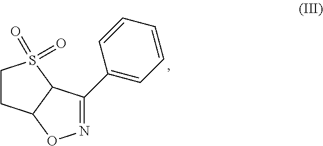 [SMILES: O=S1(=O)CCC2ON=C(c3ccccc3)C21]
and formula (IV): 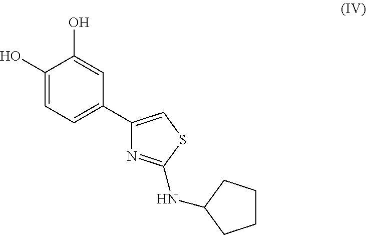 [SMILES: Oc1ccc(-c2csc(NC3CCCC3)n2)cc1O]
wherein a dashed line indicates the presence of an optional double bond, wherein W, X, Y and Z are independently selected from the group consisting of C, N, S and O,
wherein R₁, R₂, R₃, and R₄ are independently selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₅, R₆, R₇, R₈ and R₁₀ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₉ and R₁₁ are independently selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
or an N-oxide, crystalline form, hydrate thereof, or a pharmaceutically acceptable salt thereof.

28. The method of claim 27, wherein the compound is selected from the group consisting of: 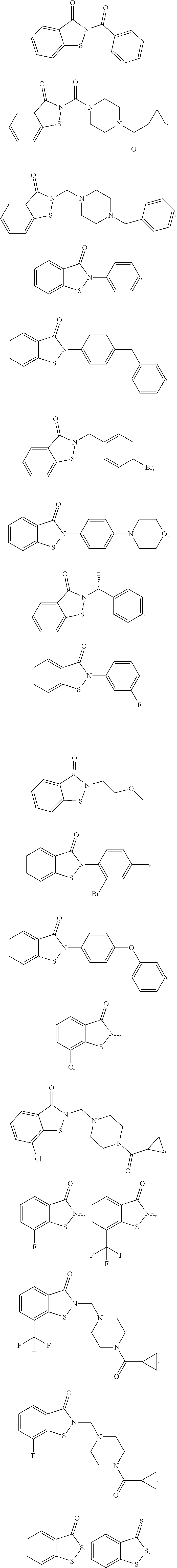
combinations thereof, or an N-oxide, crystalline form, hydrate thereof, or a pharmaceutically acceptable salt thereof.

29. The method of claim 27, wherein the subject is a mammal.

30. The method of claim 29, wherein the mammal is selected from the group consisting of humans, veterinary animals, and agricultural animals

31. The method of claim 27, wherein the subject is a human.

32. The method of claim 27, wherein the condition is selected from the group consisting of a protein folding disorder, cancer, HIV, and a blood clot.

33. A method of modulating PDI activity in a cell comprising contacting the cell with an effective amount of a compound selected from the group consisting of formula (I) 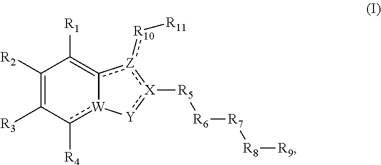
formula (II) 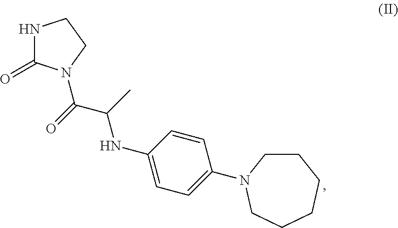 [SMILES: CC(Nc1ccc(N2CCCCCC2)cc1)C(=O)N1CCNC1=O]
formula (III) 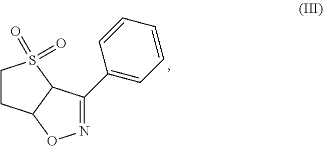 [SMILES: O=S1(=O)CCC2ON=C(c3ccccc3)C21]
and formula (IV): 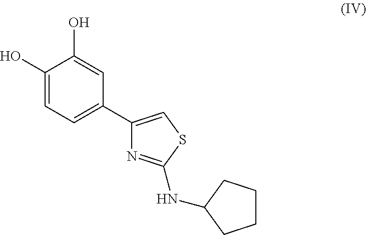 [SMILES: Oc1ccc(-c2csc(NC3CCCC3)n2)cc1O]
wherein a dashed line indicates the presence of an optional double bond, wherein W, X, Y and Z are independently selected from the group consisting of C, N, S and O,
wherein R₁, R₂, R₃, and R₄ are independently selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₅, R₆, R₇, R₈ and R₁₀ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₉ and R₁₁ are independently selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
or an N-oxide, crystalline form, hydrate thereof, or a pharmaceutically acceptable salt thereof.

34. The method of claim 33, wherein the compound is selected from the group consisting of: 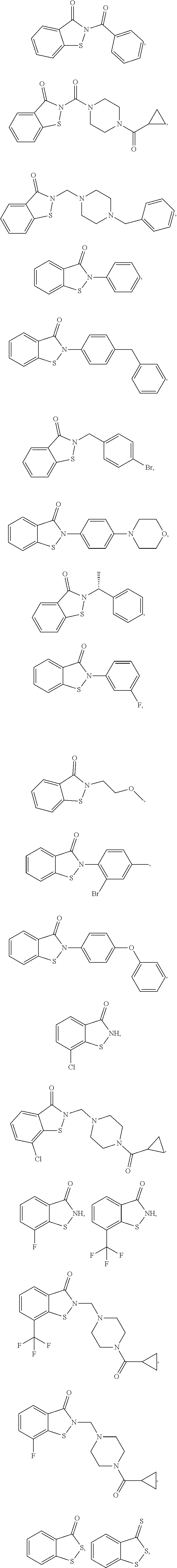
combinations thereof, or an N-oxide, crystalline form, hydrate thereof, or a pharmaceutically acceptable salt thereof.

35. A compound having the formula (I) 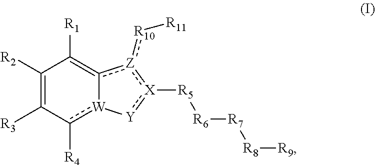
wherein a dashed line indicates the presence of an optional double bond,
wherein W, X, Y and Z are independently selected from the group consisting of C, N, S and O,
wherein R₁, R₂, R₃, and R₄ are independently selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₅, R₆, R₇, R₈ and R₁₀ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₉ and R₁₁ are independently selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
with the proviso that the compound is not 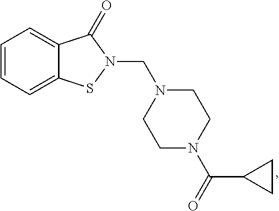 [SMILES: O=C(C1CC1)N1CCN(Cn2sc3ccccc3c2=O)CC1]
or an N-oxide, crystalline form, hydrate thereof, or a pharmaceutically acceptable salt thereof.

36. The compound of claim 35, which is selected from the group consisting of: 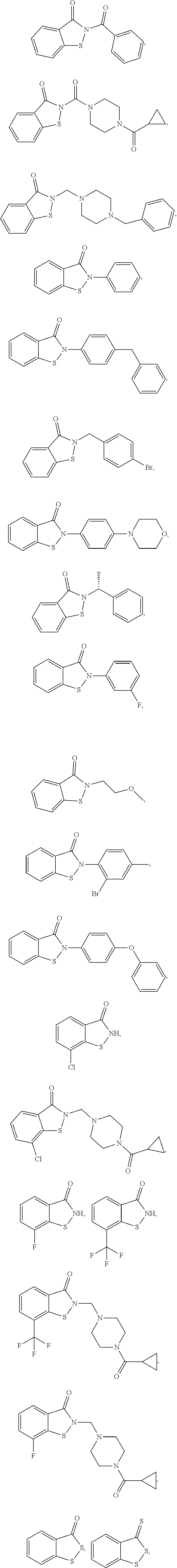
combinations thereof, or an N-oxide, crystalline form, hydrate thereof, or a pharmaceutically acceptable salt thereof.

37. A compound having the formula (Ia) 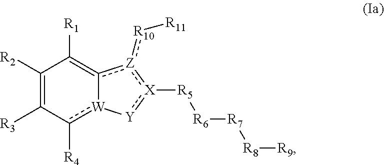
wherein a dashed line indicates the presence of an optional double bond,
wherein W, X, and Z are independently selected from the group consisting of C, N, S and O,
wherein Y is selected from the group consisting of C, N, Se, S and O,
wherein R₁, R₂, R₃, and R₄ are independently selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₅, R₆, R₇, R₈ and R₁₀ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₉ and R₁₁ are independently selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
with the proviso that the compound is not 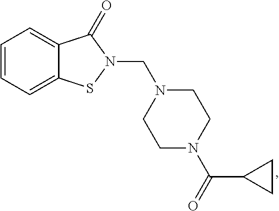 [SMILES: O=C(C1CC1)N1CCN(Cn2sc3ccccc3c2=O)CC1]
or an N-oxide, crystalline form, hydrate thereof, or a pharmaceutically acceptable salt thereof.

38. The compound of claim 37, which is selected from the group consisting of: 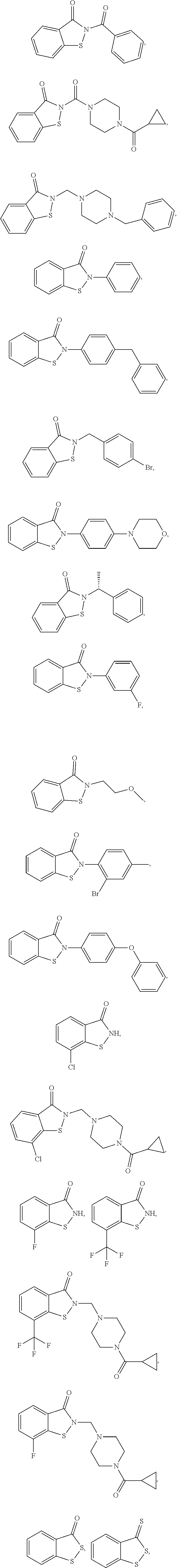
combinations thereof, or an N-oxide, crystalline form, hydrate thereof, or a pharmaceutically acceptable salt thereof.

39. A compound having the formula (II) 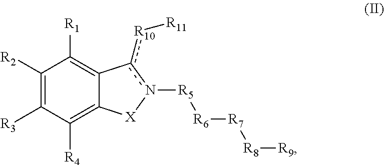
wherein a dashed line indicates the presence of an optional double bond,
wherein X is selected from the group consisting of S and Se,
wherein R₁, R₂, R₃, and R₄ are independently selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₅ is selected from the group consisting of C₁₋₄alkyl, C(O)NH, C(O), C(O)O, NH and O,
wherein R₆, is -(optionally substituted mono- or polycyclic group containing 3 to 20 carbon atoms and optionally 1 to 4 heteroatoms selected from O, N and S),
wherein R₇ and R₈ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₉ is selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₁₀ is selected from the group consisting of no atom and O,
wherein R₁₁ is selected from the group consisting of O and -(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
with the proviso that the compound is not 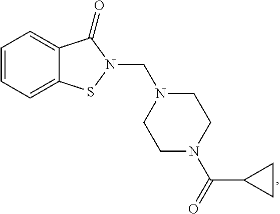 [SMILES: O=C(C1CC1)N1CCN(Cn2sc3ccccc3c2=O)CC1]
or an N-oxide, crystalline form, hydrate thereof, or a pharmaceutically acceptable salt thereof.

40. The compound of claim 39, which is selected from the group consisting of: 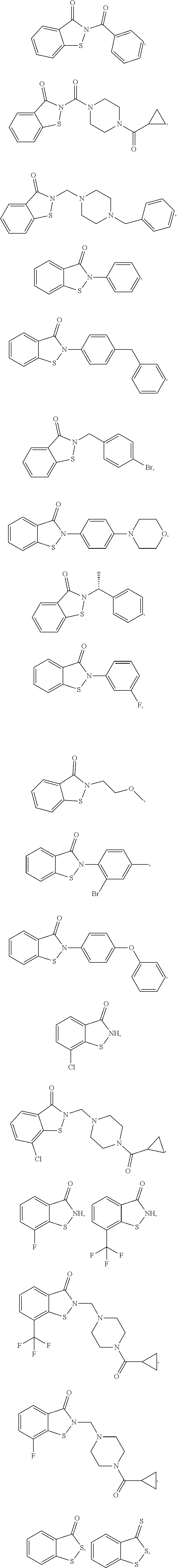
combinations thereof, or an N-oxide, crystalline form, hydrate thereof, or a pharmaceutically acceptable salt thereof.

41. The compound of claim 39, wherein the compound is 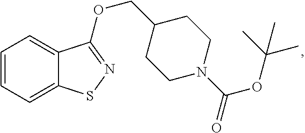 [SMILES: CC(C)(C)OC(=O)N1CCC(COc2nsc3ccccc23)CC1]
or an N-oxide, crystalline form, hydrate thereof, or a pharmaceutically acceptable salt thereof.

42. A compound having the formula (III) 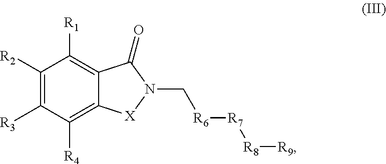
wherein X is selected from the group consisting of S and Se,
wherein R₆, is selected from the group consisting of the group consisting of phenyl, 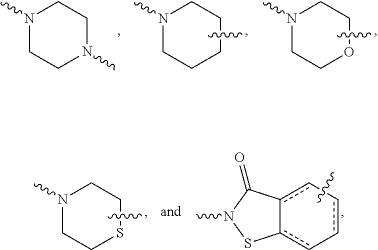
wherein a wavy line indicates an attachment point to the molecule,
wherein R₇ and R₈ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₉ is selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
with the proviso that the compound is not 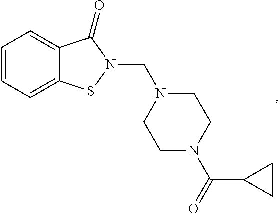 [SMILES: O=C(C1CC1)N1CCN(Cn2sc3ccccc3c2=O)CC1]
or an N-oxide, crystalline form, hydrate thereof, or a pharmaceutically acceptable salt thereof.

43. The compound of claim 42, which is selected from the group consisting of: 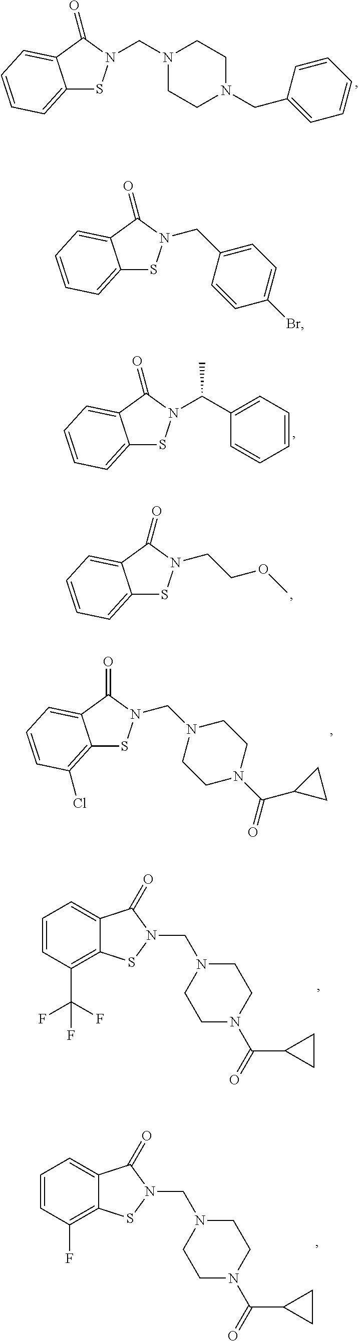
combinations thereof, or an N-oxide, crystalline form, hydrate thereof, or a pharmaceutically acceptable salt thereof.

44. A compound of claim 37, which is selected from the group consisting of 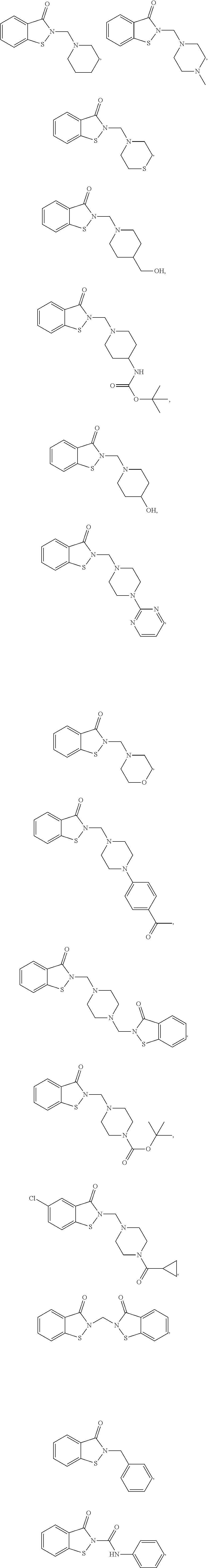
and combinations thereof.

45. A composition comprising a compound according to any one of claims 35-44 and a pharmaceutically acceptable carrier, adjuvant or vehicle.

46. A pharmaceutically acceptable salt of a compound according to any one of claims 35-44.

47. A composition comprising a pharmaceutically acceptable salt of a compound according to any one of claims 35-44 and a pharmaceutically acceptable carrier, adjuvant or vehicle.

48. A kit comprising:
i) a compound according to any one of claims 35-44 or a composition according to claim 45; and
ii) instructions for use.

49. The kit according to claim 48, wherein the instructions for use are instructions for treating or ameliorating the effects of a neurodegenerative disorder in a subject.

50. The kit according to claim 48, wherein the instructions for use are instructions for treating or ameliorating the effects of a condition associated with increased protein disulfide isomerase (PDI) activity in a subject.

51. The kit according to claim 48, wherein the instructions for use are instructions for modulating PDI activity in a cell.

52. A method for treating or ameliorating the effects of a neurodegenerative disorder in a subject in need thereof comprising administering to the subject an effective amount of a compound selected from the group consisting of formula (Ia) 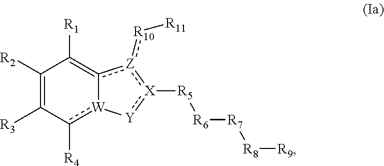
wherein a dashed line indicates the presence of an optional double bond,
wherein W, X, and Z are independently selected from the group consisting of C, N, S and O,
wherein Y is selected from the group consisting of C, N, Se, S and O,
wherein R₁, R₂, R₃, and R₄ are independently selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₅, R₆, R₇, R₈ and R₁₀ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₉ and R₁₁ are independently selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
or an N-oxide, crystalline form, hydrate thereof, or a pharmaceutically acceptable salt thereof;
formula (II) 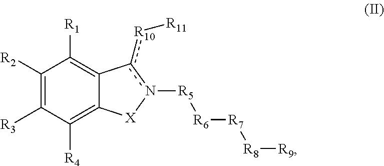
wherein a dashed line indicates the presence of an optional double bond,
wherein X is selected from the group consisting of S and Se,
wherein R₁, R₂, R₃, and R₄ are independently selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₅ is selected from the group consisting of C₁₋₄alkyl, C(O)NH, C(O), C(O)O, NH and O,
wherein R₆, is -(optionally substituted mono- or polycyclic group containing 3 to 20 carbon atoms and optionally 1 to 4 heteroatoms selected from O, N and S),
wherein R₇ and R₈ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₉ is selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₁₀ is selected from the group consisting of no atom and O,
wherein R₁₁ is selected from the group consisting of O and -(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
or an N-oxide, crystalline form, hydrate thereof, or a pharmaceutically acceptable salt thereof; and 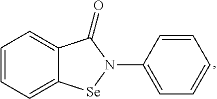 [SMILES: O=c1c2ccccc2[se]n1-c1ccccc1]
or an N-oxide, crystalline form, hydrate thereof, or a pharmaceutically acceptable salt thereof.

53. The method of claim 52, wherein the compound is selected from the group consisting of: 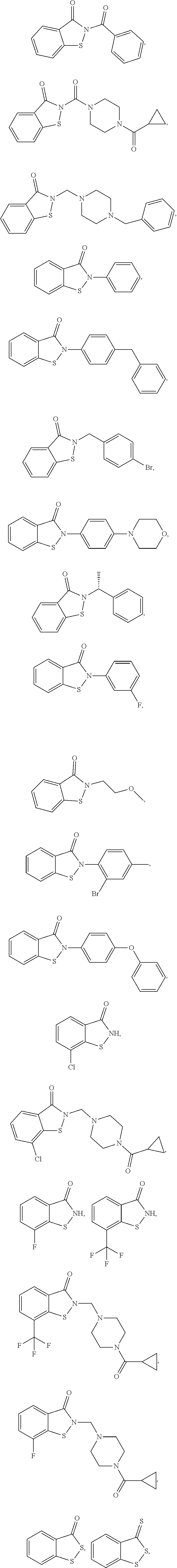
combinations thereof, or an N-oxide, crystalline form, hydrate thereof, or a pharmaceutically acceptable salt thereof.

54. A method for treating or ameliorating the effects of a neurodegenerative disorder in a subject in need thereof comprising administering to the subject an effective amount of a composition of claim 45 or claim 47.

55. The method of claim 52 or claim 54, wherein the neurodegenerative disease is selected from the group consisting of Alzheimer's disease, Parkinson's disease, amyotrophic lateral sclerosis (ALS), Friedreich's ataxia, multiple sclerosis, Huntington's Disease, transmissible spongiform encephalopathy, Charcot-Marie-Tooth disease, dementia with Lewy bodies, corticobasal degeneration, progressive supranuclear palsy, and hereditary spastic paraparesis.

56. The method of claim 52 or claim 54, wherein the neurodegenerative disease is Huntington's Disease.

57. The method of claim 52 or claim 54, wherein the subject is a mammal.

58. The method of claim 52 or claim 54, wherein the mammal is selected from the group consisting of humans, veterinary animals, and agricultural animals.

59. The method of claim 52 or claim 54, wherein the subject is a human.

60. The method of claim 52 or claim 54, further comprising co-administering to the subject an effective amount of one or more additional therapeutic agents.

61. The method of claim 52 or claim 54, wherein the one or more additional therapeutic agents are selected from the group consisting of 5-hydroxytryptophan, Activase, AFQ056 (Novartis), Aggrastat, Albendazole, alpha-lipoic acid/L-acetyl carnitine, Alteplase, Amantadine (Symmetrel), amlodipine, Ancrod, Apomorphine (Apokyn), Arimoclomol, Arixtra, Armodafinil, Ascorbic acid, Ascriptin, Aspirin, atenolol, Avonex, baclofen (Lioresal), Banzel, Benztropine (Cogentin), Betaseron, BGG492 (Novartis Corp.), Botulinum toxin, Bufferin, Carbatrol®, Carbidopa/levodopa immediate-release (Sinemet), Carbidopa/levodopa oral disintegrating (Parcopa), Carbidopa/levodopa/Entacapone (Stalevo), CERE-110: Adeno-Associated Virus Delivery of NGF (Ceregene), cerebrolysin, CinnoVex, citalopram, citicoline, Clobazam, Clonazepam, Clopidogrel, clozapine (Clozaril), Coenzyme Q, Creatine, dabigatran, dalteparin, Dapsone, Davunetide, Deferiprone, Depakene®, Depakote ER®, Depakote®, Desmoteplase, Diastat, Diazepam, Digoxin, Dilantin®, Dimebon, dipyridamole, divalproex (Depakote), Donepezil (Aricept), EGb 761, Eldepryl, ELND002 (Elan Pharmaceuticals), Enalapril, enoxaparin, Entacapone (Comtan), epoetin alfa, Eptifibatide, Erythropoietin, Escitalopram, Eslicarbazepine acetate, Esmolol, Ethosuximide, Ethyl-EPA (Miraxion™), Exenatide, Extavia, Ezogabine, Felbamate, Felbatol®, Fingolimod (Gilenya), fluoxetine (Prozac), fondaparinux, Fragmin, Frisium, Gabapentin, Gabitril®, Galantamine, Glatiramer (Copaxone), haloperidol (Haldol), Heparin, human chorionic gonadotropin (hCG), Idebenone, Inovelon®, insulin, Interferon beta 1a, Interferon beta 1b, ioflupane 1231 (DATSCAN®), IPX066 (Impax Laboratories Inc.), JNJ-26489112 (Johnson and Johnson), Keppra®, Klonopin, Lacosamide, L-Alpha glycerylphosphorylcholine, Lamictal®, Lamotrigine, Levetiracetam, liraglutide, Lisinopril, Lithium carbonate, Lopressor, Lorazepam, losartan, Lovenox, Lu AA24493, Luminal, LY450139 (Eli Lilly), Lyrica, Masitinib, Mecobalamin, Memantine, methylprednisolone, metoprolol tartrate, Minitran, Minocycline, mirtazapine, Mitoxantrone (Novantrone), Mysoline®, Natalizumab (Tysabri), Neurontin®, Niacinamide, Nitro-Bid, Nitro-Dur, nitroglycerin, Nitrolingual, Nitromist, Nitrostat, Nitro-Time, Norepinephrine (NOR), Carbamazepine, octreotide, Onfi®, Oxcarbazepine, Oxybutinin chloride, PF-04360365 (Pfizer), Phenobarbital, Phenytek®, Phenytoin, piclozotan, Pioglitazone, Plavix, Potiga, Pramipexole (Mirapex), pramlintide, Prednisone, Primidone, Prinivil, probenecid, Propranolol, PRX-00023 (EPIX Pharmaceuticals Inc.), PXT3003, Quinacrine, Ramelteon, Rasagiline (Azilect), Rebif, ReciGen, remacemide, Resveratrol, Retavase, reteplase, riluzole (Rilutek), Rivastigmine (Exelon), Ropinirole (Requip), Rotigotine (Neupro), Rufinamide, Sabril, safinamide (EMD Serono), Salagen, Sarafem, Selegiline (1-deprenyl, Eldepryl), SEN0014196 (Siena Biotech), sertraline (Zoloft), Simvastatin, Sodium Nitroprussiate (NPS), sodium phenylbutyrate, Stanback Headache Powder, Tacrine (Cognex), Tamoxifen, tauroursodeoxycholic acid (TUDCA), Tegretol®, Tenecteplase, Tenormin, Tetrabenazine (Xenazine), THR-18 (Thrombotech Ltd.), Tiagabine, Tideglusib, tirofiban, tissue plasminogen activator (tPA), tizanidine (Zanaflex), TNKase, Tolcapone (Tasmar), Tolterodine, Topamax®, Topiramate, Trihexyphenidyl (formerly Artane), Trileptal®, ursodiol, Valproic Acid, valsartan, Varenicline (Pfizer), Vimpat, Vitamin E, Warfarin, Zarontin®, Zestril, Zonegran®, Zonisamide, Zydis selegiline HCL Oral disintegrating (Zelapar), and combinations thereof.

62. The method of claim 52, wherein the compound is selected from the group consisting of 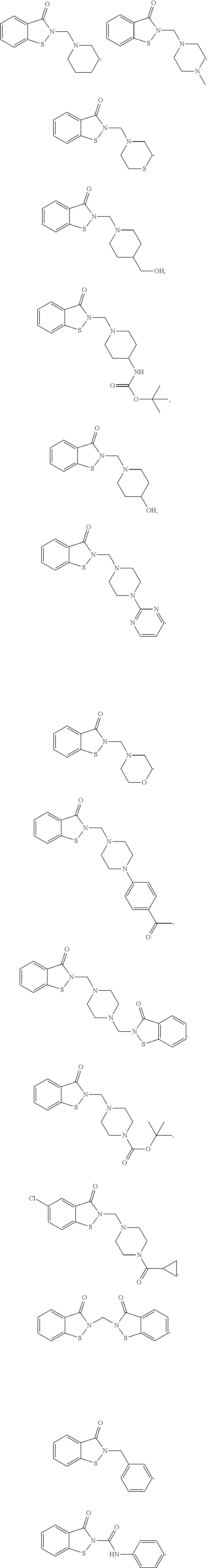
and combinations thereof.

63. A method for treating or ameliorating the effects of a condition associated with increased protein disulfide isomerase (PDI) activity in a subject in need thereof comprising administering to the subject an effective amount of a compound selected from the group consisting of formula (Ia) 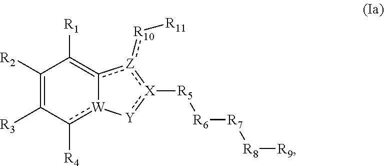
wherein a dashed line indicates the presence of an optional double bond,
wherein W, X, and Z are independently selected from the group consisting of C, N, S and O,
wherein Y is selected from the group consisting of C, N, Se, S and O,
wherein R₁, R₂, R₃, and R₄ are independently selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₅, R₆, R₇, R₈ and R₁₀ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₉ and R₁₁ are independently selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
or an N-oxide, crystalline form, hydrate thereof, or a pharmaceutically acceptable salt thereof;
formula (II) 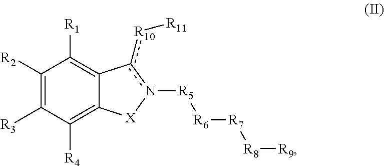
wherein a dashed line indicates the presence of an optional double bond,
wherein X is selected from the group consisting of S and Se,
wherein R₁, R₂, R₃, and R₄ are independently selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₅ is selected from the group consisting of C₁₋₄alkyl, C(O)NH, C(O), C(O)O, NH and O,
wherein R₆, is -(optionally substituted mono- or polycyclic group containing 3 to 20 carbon atoms and optionally 1 to 4 heteroatoms selected from O, N and S),
wherein R₇ and R₈ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₉ is selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₁₀ is selected from the group consisting of no atom and O,
wherein R₁₁ is selected from the group consisting of O and -(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
or an N-oxide, crystalline form, hydrate thereof, or a pharmaceutically acceptable salt thereof; and 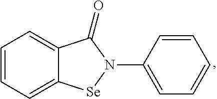 [SMILES: O=c1c2ccccc2[se]n1-c1ccccc1]
or an N-oxide, crystalline form, hydrate thereof, or a pharmaceutically acceptable salt thereof.

64. The method of claim 63, wherein the compound is selected from the group consisting of: 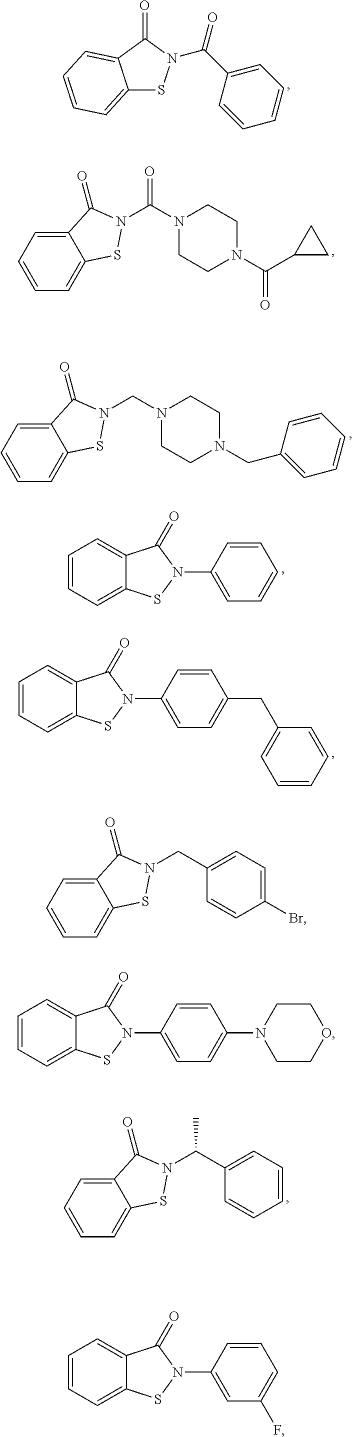 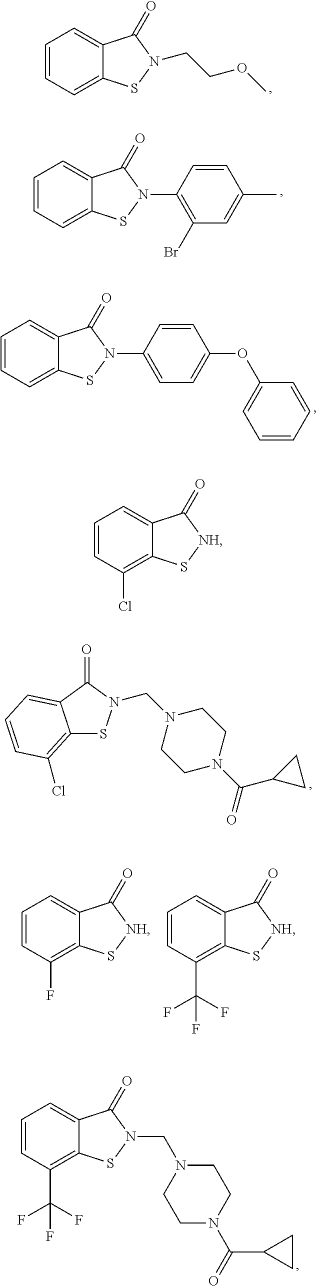 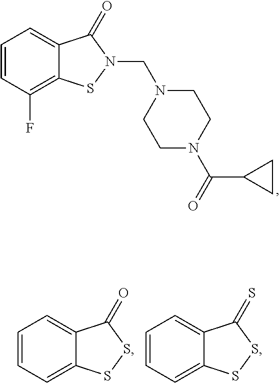
combinations thereof, or an N-oxide, crystalline form, hydrate thereof, or a pharmaceutically acceptable salt thereof.

65. A method for treating or ameliorating the effects of a condition associated with increased protein disulfide isomerase (PDI) activity in a subject in need thereof comprising administering to the subject an effective amount of a composition of claim 45 or claim 47.

66. The method of claim 63 or claim 65, wherein the subject is a mammal.

67. The method of claim 63 or claim 65, wherein the mammal is selected from the group consisting of humans, veterinary animals, and agricultural animals.

68. The method of claim 63 or claim 65, wherein the subject is a human.

69. The method of claim 63 or claim 65, wherein the condition is selected from the group consisting of a protein folding disorder, cancer, HIV, and a blood clot.

70. The method of claim 63, wherein the compound is selected from the group consisting of 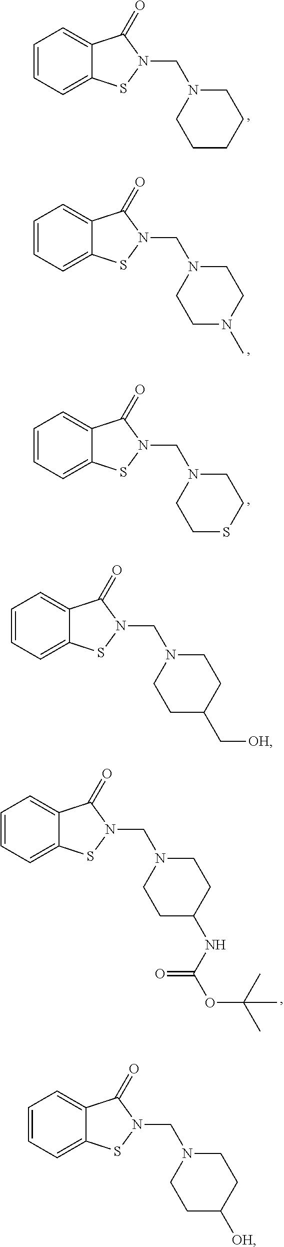 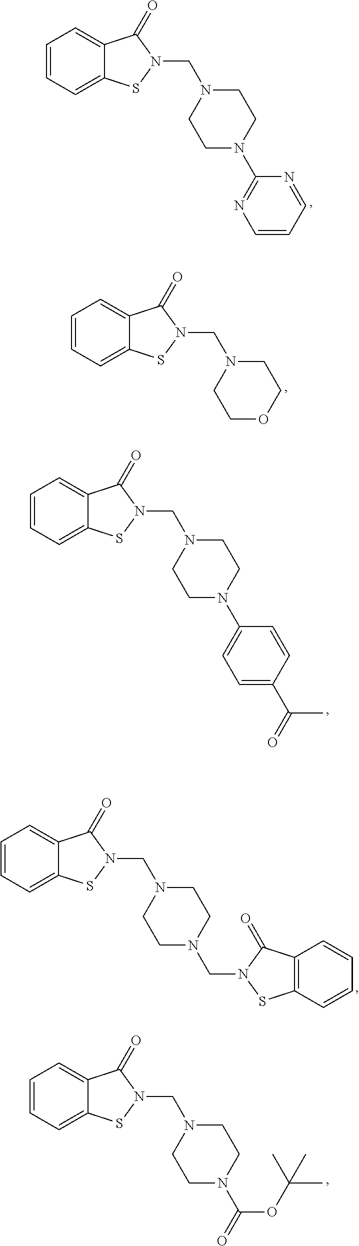 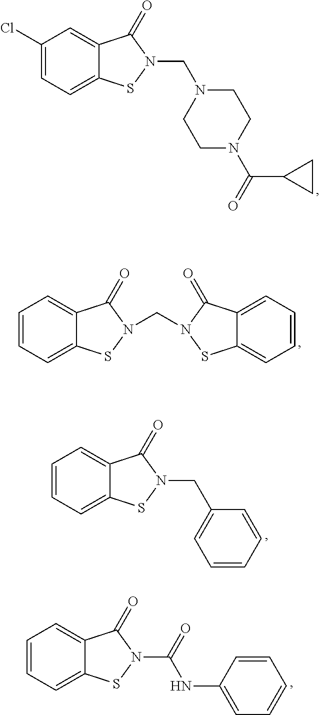
and combinations thereof.

71. A method of modulating PDI activity in a cell comprising administering to the subject an effective amount of a compound selected from the group consisting of formula (Ia) 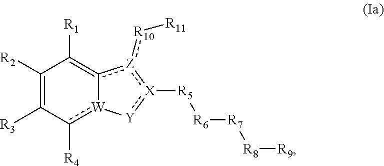
wherein a dashed line indicates the presence of an optional double bond,
wherein W, X, and Z are independently selected from the group consisting of C, N, S and O,
wherein Y is selected from the group consisting of C, N, Se, S and O,
wherein R₁, R₂, R₃, and R₄ are independently selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₅, R₆, R₇, R₈ and R₁₀ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₉ and R₁₁ are independently selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
or an N-oxide, crystalline form, hydrate thereof, or a pharmaceutically acceptable salt thereof;
formula (II) 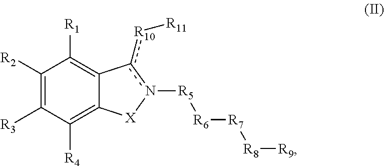
wherein a dashed line indicates the presence of an optional double bond,
wherein X is selected from the group consisting of S and Se,
wherein R₁, R₂, R₃, and R₄ are independently selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₅ is selected from the group consisting of C₁₋₄alkyl, C(O)NH, C(O), C(O)O, NH and O,
wherein R₆, is -(optionally substituted mono- or polycyclic group containing 3 to 20 carbon atoms and optionally 1 to 4 heteroatoms selected from O, N and S),
wherein R₇ and R₈ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₉ is selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₁₀ is selected from the group consisting of no atom and O,
wherein R₁₁ is selected from the group consisting of O and -(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
or an N-oxide, crystalline form, hydrate thereof, or a pharmaceutically acceptable salt thereof; and 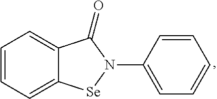 [SMILES: O=c1c2ccccc2[se]n1-c1ccccc1]
or an N-oxide, crystalline form, hydrate thereof, or a pharmaceutically acceptable salt thereof.

72. The method of claim 71, wherein the compound is selected from the group consisting of: 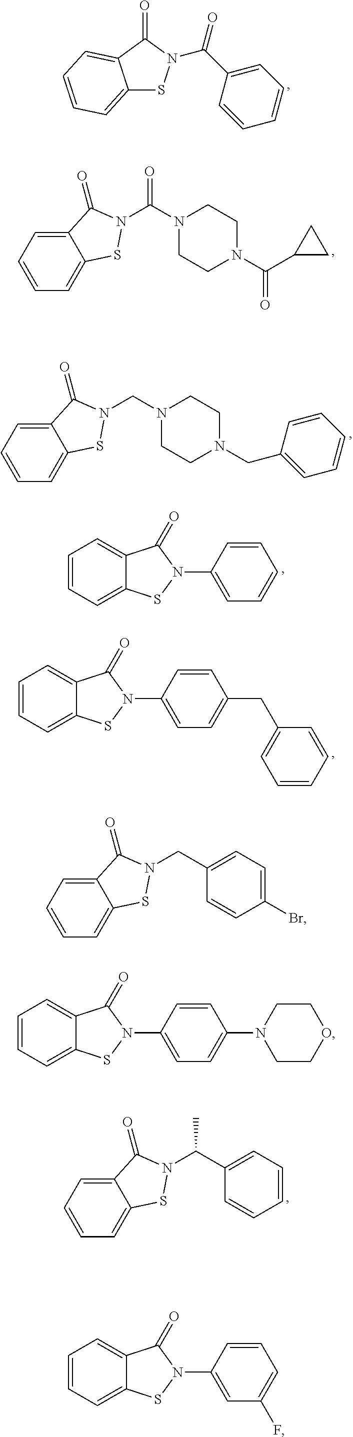 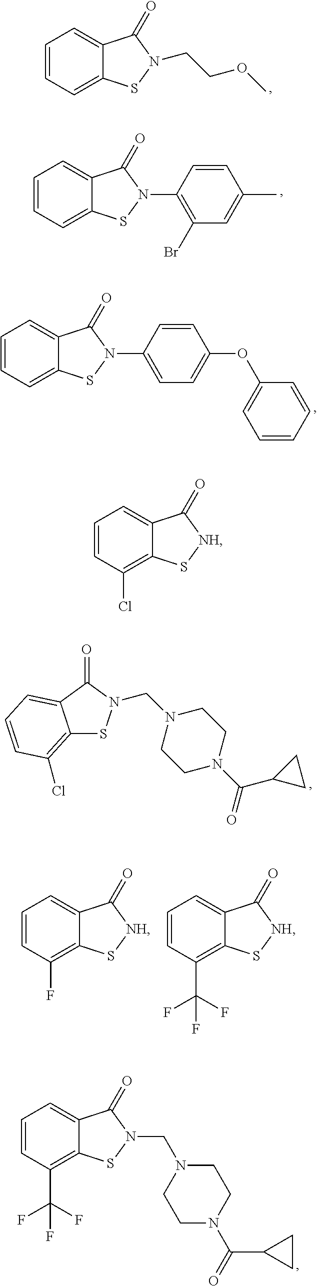 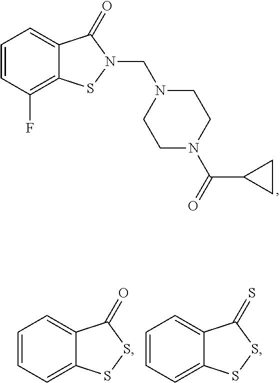
combinations thereof, or an N-oxide, crystalline form, hydrate thereof, or a pharmaceutically acceptable salt thereof.

73. A method of modulating PDI activity in a cell comprising administering to the subject an effective amount of a composition of claim 45 or claim 47.

74. The method of claim 71, wherein the compound is selected from the group consisting of 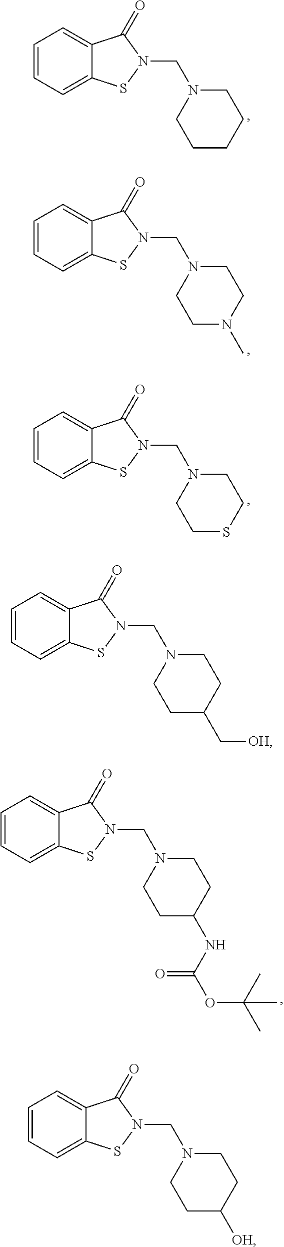 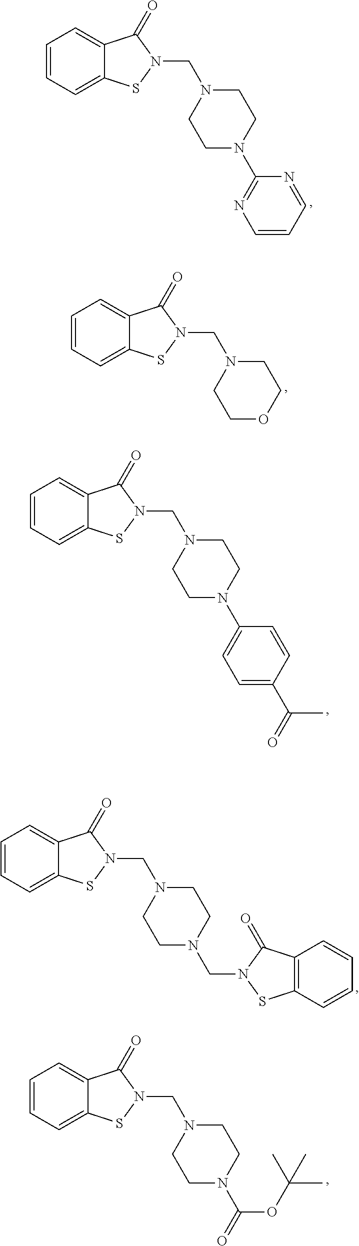 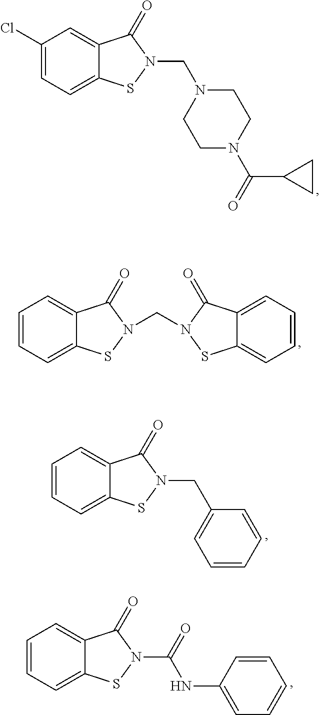
and combinations thereof.

75. A method for treating or ameliorating the effects of a neurodegenerative disorder in a subject in need thereof comprising administering to the subject an effective amount of a compound selected from the group consisting of: 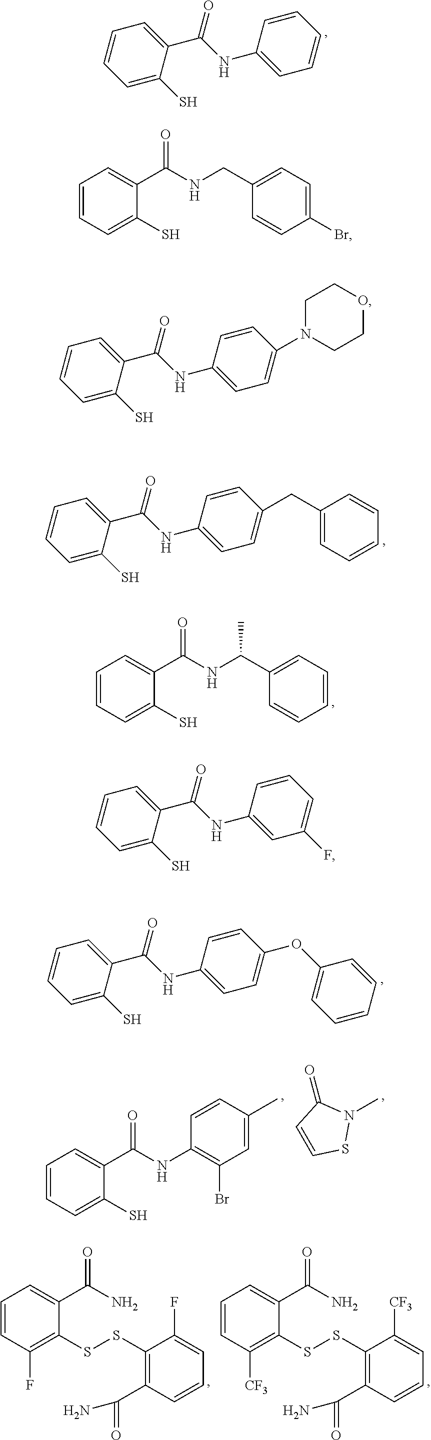 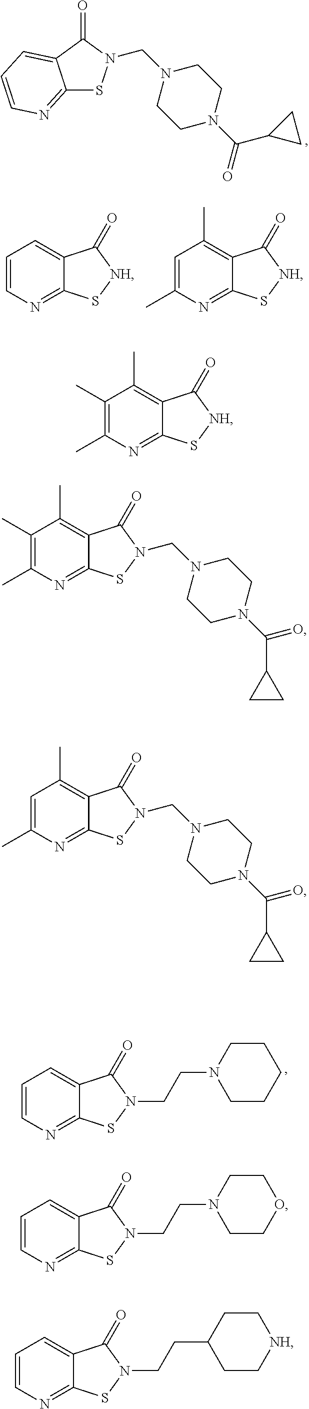 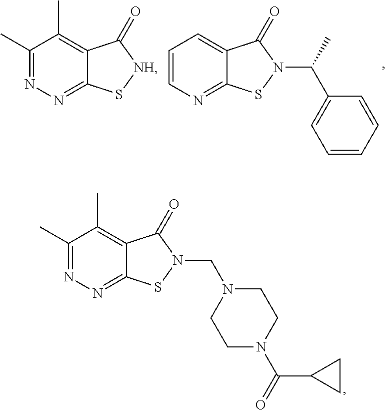
combinations thereof,
or an N-oxide, crystalline form, hydrate thereof, or a pharmaceutically acceptable salt thereof.

76. The method of claim 75, wherein the neurodegenerative disease is selected from the group consisting of Alzheimer's disease, Parkinson's disease, amyotrophic lateral sclerosis (ALS), Friedreich's ataxia, multiple sclerosis, Huntington's Disease, transmissible spongiform encephalopathy, Charcot-Marie-Tooth disease, dementia with Lewy bodies, corticobasal degeneration, progressive supranuclear palsy, and hereditary spastic paraparesis.

77. The method of claim 75, wherein the neurodegenerative disease is Huntington's Disease.

78. The method of claim 75, wherein the subject is a mammal.

79. The method of claim 78, wherein the mammal is selected from the group consisting of humans, veterinary animals, and agricultural animals.

80. The method of claim 75, wherein the subject is a human.

81. The method of claim 75, further comprising co-administering to the subject an effective amount of one or more additional therapeutic agents.

82. The method of claim 81, wherein the one or more additional therapeutic agents are selected from the group consisting of 5-hydroxytryptophan, Activase, AFQ056 (Novartis), Aggrastat, Albendazole, alpha-lipoic acid/L-acetyl carnitine, Alteplase, Amantadine (Symmetrel), amlodipine, Ancrod, Apomorphine (Apokyn), Arimoclomol, Arixtra, Armodafinil, Ascorbic acid, Ascriptin, Aspirin, atenolol, Avonex, baclofen (Lioresal), Banzel, Benztropine (Cogentin), Betaseron, BGG492 (Novartis Corp.), Botulinum toxin, Bufferin, Carbatrol®, Carbidopa/levodopa immediate-release (Sinemet), Carbidopa/levodopa oral disintegrating (Parcopa), Carbidopa/levodopa/Entacapone (Stalevo), CERE-110: Adeno-Associated Virus Delivery of NGF (Ceregene), cerebrolysin, CinnoVex, citalopram, citicoline, Clobazam, Clonazepam, Clopidogrel, clozapine (Clozaril), Coenzyme Q, Creatine, dabigatran, dalteparin, Dapsone, Davunetide, Deferiprone, Depakene®, Depakote ER®, Depakote®, Desmoteplase, Diastat, Diazepam, Digoxin, Dilantin®, Dimebon, dipyridamole, divalproex (Depakote), Donepezil (Aricept), EGb 761, Eldepryl, ELND002 (Elan Pharmaceuticals), Enalapril, enoxaparin, Entacapone (Comtan), epoetin alfa, Eptifibatide, Erythropoietin, Escitalopram, Eslicarbazepine acetate, Esmolol, Ethosuximide, Ethyl-EPA (Miraxion™), Exenatide, Extavia, Ezogabine, Felbamate, Felbatol®, Fingolimod (Gilenya), fluoxetine (Prozac), fondaparinux, Fragmin, Frisium, Gabapentin, Gabitril®, Galantamine, Glatiramer (Copaxone), haloperidol (Haldol), Heparin, human chorionic gonadotropin (hCG), Idebenone, Inovelon®, insulin, Interferon beta 1a, Interferon beta 1b, ioflupane 1231 (DATSCAN®), IPX066 (Impax Laboratories Inc.), JNJ-26489112 (Johnson and Johnson), Keppra®, Klonopin, Lacosamide, L-Alpha glycerylphosphorylcholine, Lamictal®, Lamotrigine, Levetiracetam, liraglutide, Lisinopril, Lithium carbonate, Lopressor, Lorazepam, losartan, Lovenox, Lu AA24493, Luminal, LY450139 (Eli Lilly), Lyrica, Masitinib, Mecobalamin, Memantine, methylprednisolone, metoprolol tartrate, Minitran, Minocycline, mirtazapine, Mitoxantrone (Novantrone), Mysoline®, Natalizumab (Tysabri), Neurontin®, Niacinamide, Nitro-Bid, Nitro-Dur, nitroglycerin, Nitrolingual, Nitromist, Nitrostat, Nitro-Time, Norepinephrine (NOR), Carbamazepine, octreotide, Onfi®, Oxcarbazepine, Oxybutinin chloride, PF-04360365 (Pfizer), Phenobarbital, Phenytek®, Phenytoin, piclozotan, Pioglitazone, Plavix, Potiga, Pramipexole (Mirapex), pramlintide, Prednisone, Primidone, Prinivil, probenecid, Propranolol, PRX-00023 (EPIX Pharmaceuticals Inc.), PXT3003, Quinacrine, Ramelteon, Rasagiline (Azilect), Rebif, ReciGen, remacemide, Resveratrol, Retavase, reteplase, riluzole (Rilutek), Rivastigmine (Exelon), Ropinirole (Requip), Rotigotine (Neupro), Rufinamide, Sabril, safinamide (EMD Serono), Salagen, Sarafem, Selegiline (1-deprenyl, Eldepryl), SEN0014196 (Siena Biotech), sertraline (Zoloft), Simvastatin, Sodium Nitroprussiate (NPS), sodium phenylbutyrate, Stanback Headache Powder, Tacrine (Cognex), Tamoxifen, tauroursodeoxycholic acid (TUDCA), Tegretol®, Tenecteplase, Tenormin, Tetrabenazine (Xenazine), THR-18 (Thrombotech Ltd.), Tiagabine, Tideglusib, tirofiban, tissue plasminogen activator (tPA), tizanidine (Zanaflex), TNKase, Tolcapone (Tasmar), Tolterodine, Topamax®, Topiramate, Trihexyphenidyl (formerly Artane), Trileptal®, ursodiol, Valproic Acid, valsartan, Varenicline (Pfizer), Vimpat, Vitamin E, Warfarin, Zarontin®, Zestril, Zonegran®, Zonisamide, Zydis selegiline HCL Oral disintegrating (Zelapar), and combinations thereof.

83. A method for treating or ameliorating the effects of a condition associated with increased protein disulfide isomerase (PDI) activity in a subject in need thereof comprising administering to the subject an effective amount of a compound selected from the group consisting of: 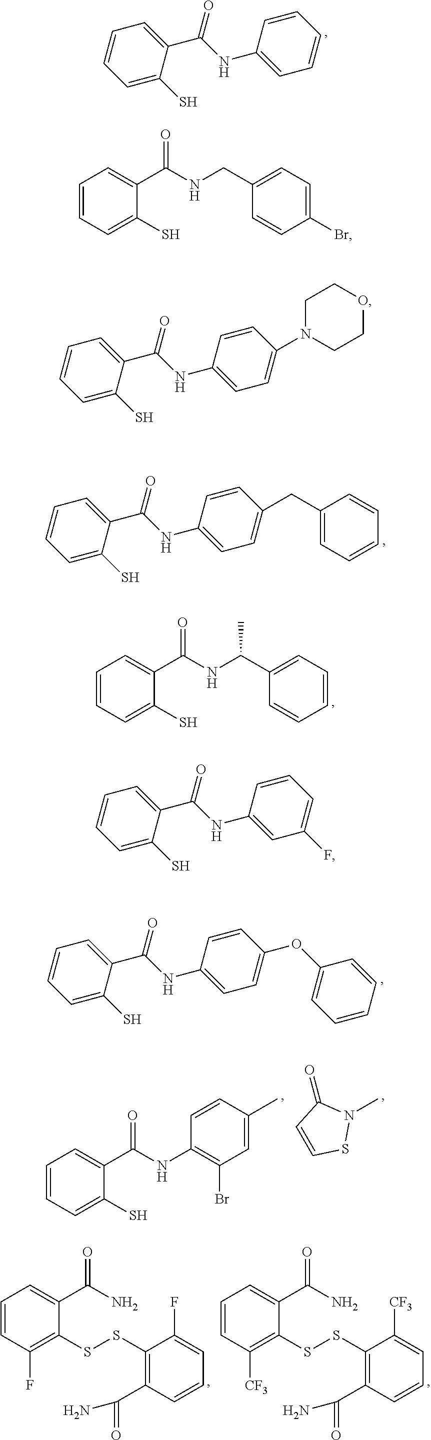 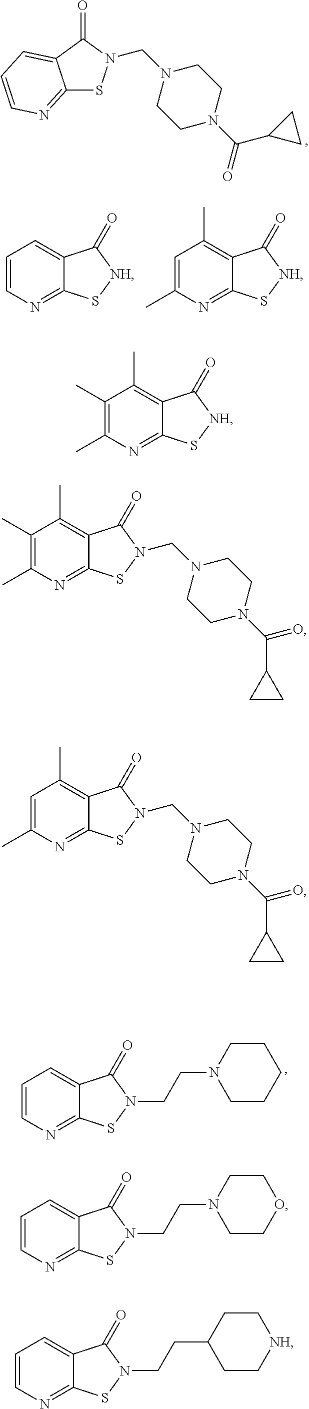 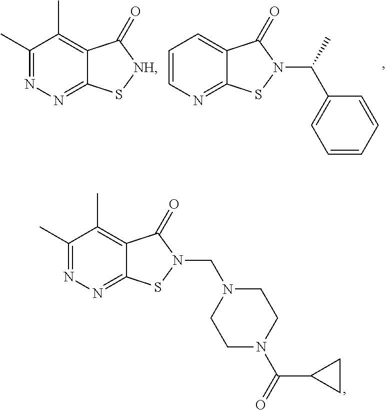
combinations thereof,
or an N-oxide, crystalline form, hydrate thereof, or a pharmaceutically acceptable salt thereof.

84. A method of modulating PDI activity in a cell comprising contacting the cell with an effective amount of a compound selected from the group consisting of: 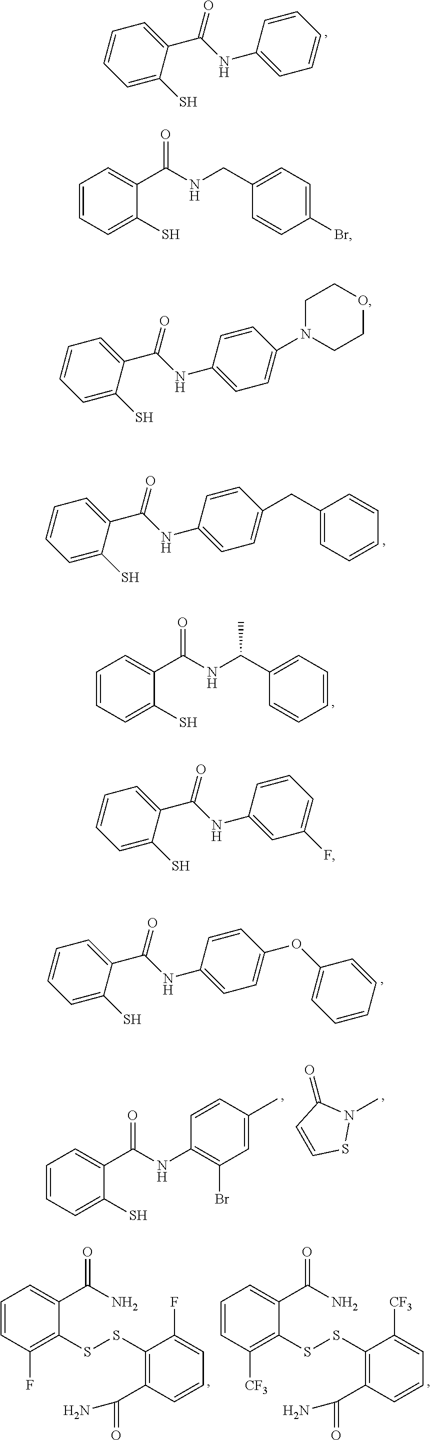 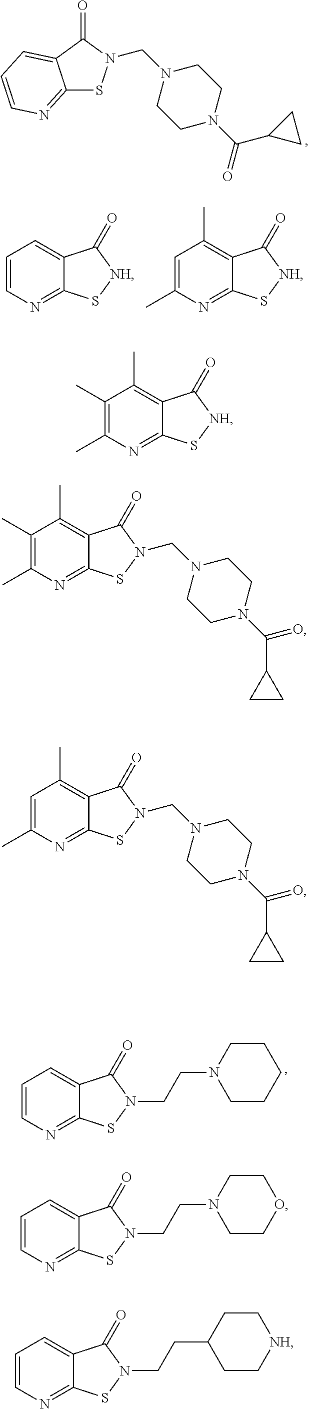 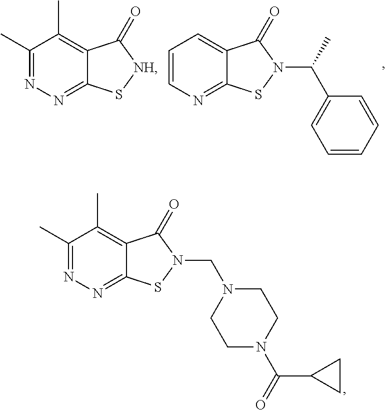
combinations thereof,
or an N-oxide, crystalline form, hydrate thereof, or a pharmaceutically acceptable salt thereof.

85. A compound selected from the group consisting of: 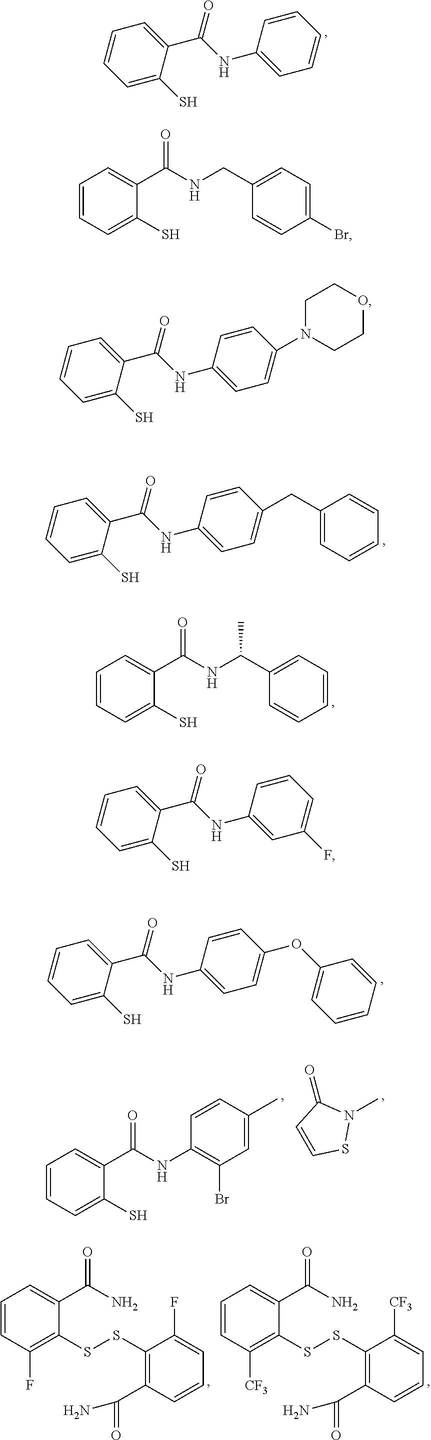 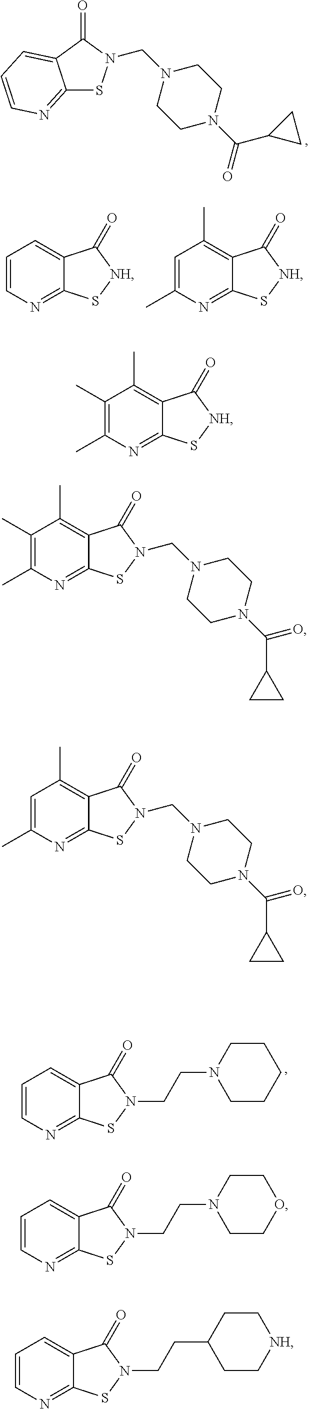 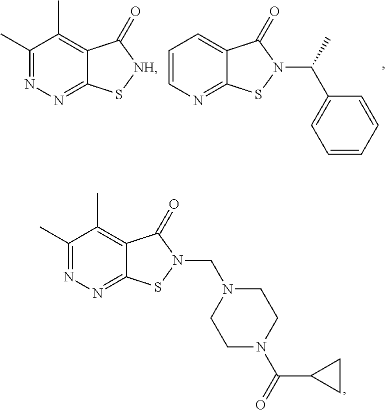
combinations thereof,
or an N-oxide, crystalline form, hydrate thereof, or a pharmaceutically acceptable salt thereof.

86. A composition comprising a compound according to claim 85 and a pharmaceutically acceptable carrier, adjuvant or vehicle.
